(19) United States
(12) Patent Application Publication
(10) Pub. No.: US 2021/0371385 A1
(43) Pub. Date: Dec. 2, 2021

(54) POLYMORPHIC AND AMORPHOUS FORMS OF ISOQUINOLINONE AND METHODS OF USE THEREOF

(71) Applicant: Sojournix, Inc., Waltham, MA (US)

(72) Inventors: Jette Bisgaard Boll, Ballerup (DK); Jayachandra P. Reddy, West Palm Beach, FL (US)

(21) Appl. No.: 17/050,807

(22) PCT Filed: Apr. 29, 2019
(86) PCT No.: PCT/US19/29751

§ 371 Date: Oct. 26, 2020

Related U.S. Application Data
(60) Provisional application No. 62/664,020, filed on Apr. 27, 2018.

(51) Int. Cl. C07D 217/26

(52) U.S. Cl. CPC C07D 217/26; C07B 2200/13

(57) ABSTRACT

Polymorphic and amorphous forms of 2-ethylamino-8-fluoro-3 -methyl-1-oxo-1,2-dihydro-isoquinoline-4-carboxylic acid ((S)-cyclopropyl-phenyl-methyl)-amide (also referred to herein as SJB-01). The polymorphic forms may be an alpha, a beta, or a gamma form.

### CROSS-REFERENCE TO RELATED APPLICATIONS

This application claims the benefit of U.S. Provisional Application No. 62/664,020, filed on Apr. 27, 2018. The entire teachings of the above application are incorporated herein by reference.

### BACKGROUND OF THE INVENTION

Recently, neurokinin receptors have been suggested as targets for CNS diseases (Albert, Expert Opin. Ther. Patents, 14, 1421-1433, 2004). Neurokinins (or tachykinins) are a family of neuropeptides which include substance P (SP), neurokinin A (NKA), and neurokinin B (NKB). The biological effects of these substances are primarily effected through binding to and activation of the three neurokinin receptors NK1, NK2, and NK3. Although some cross reactivity probably exists, SP has the highest affinity and is believed to be the endogenous ligand for NK1. Similarly, NKA is believed to be the endogenous ligand for NK2, and NKB is believed to be the endogenous ligand for NK3.

NK3 is primarily expressed centrally in regions including cortical regions, such as frontal, parietal and cingulated cortex; nuclei of the amygdale, such as the basal, central and lateral nuclei; the hippocampus; and mesencephalon structures, such as ventral tegmental area, substantia nigra pars compacta, and dorsal raphe nuclei (Spooren et al, Nature Reviews, 4, 967-975, 2005). In addition, morphological studies in rats have provided evidence for putative interactions between NKB neurons and the hypothalamic reproductive axis (Krajewski, J. Comp. Neurol., 489, 372-386, 2005). NKB expression is shown to co-localize with estrogen receptor a and dynorphin in arcuate nucleus neurons (Burke, J. Comp. Neurol., 498, 712-726, 2006; Goodman, Endocrinology, 145, 2959-296, 2004). Further, the NK3 receptor is highly expressed in the hypothalamic arcuate nucleus in neurons, which are involved in the regulation of Gonadotrophin Releasing Hormone (GnRH) release.

Activation of NK-3 receptors has been shown to modulate dopamine, acetylcholine, and serotonin release thereby suggesting a therapeutic utility for NK3 receptor modulators for the treatment of a variety of disorders including psychotic disorders, anxiety, depression, schizophrenia, obesity, pain, and inflammation (Giardina, Expert Opin. Ther. Patents, 10, 939-960, 2000). NK3 receptor modulators may also have therapeutic utility for treating sex hormone-dependent diseases.

New potent and selective antagonists of the NK3 receptor may be of therapeutic value for the preparation of drugs useful in the treatment and/or prevention of a number of diseases or conditions in which NKB and the NK3 receptor are involved.

### SUMMARY OF THE INVENTION

In some aspects, the present disclosure relates to a polymorph of a compound of formula (I):

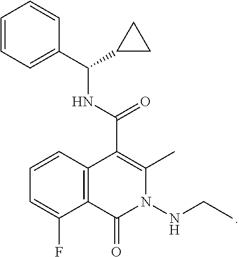 [SMILES: CCNn1c(C)c(C(=O)N[C@H](c2ccccc2)C2CC2)c2cccc(F)c2c1=O]

In some embodiments, the polymorph is an α form, a β form, or a γ form. In some embodiments, the polymorph is a substantially pure α form, a substantially pure β form, or a substantially pure γ form

In some embodiments, the polymorph (e.g., an alpha form) has an X-ray powder diffraction pattern comprising a peak at about 6.37° 2θ. In some embodiments, the polymorph (e.g., an alpha form) has an X-ray powder diffraction pattern comprising peaks at about 6.37° and 8.60° 2θ. In some embodiments, the polymorph (e.g., an alpha form) has an X-ray powder diffraction pattern comprising peaks at about 6.37°, 8.60°, 9.06°, 10.72°, 11.76°, 12.71°, 14.62°, 17.13°, 19.74°, 23.02°, and 24.99° 2θ. In some embodiments, the polymorph (e.g., an alpha form) has an X-ray powder diffraction pattern substantially as shown in FIG. 33

In some embodiments, the polymorph (e.g., a beta form) has an X-ray powder diffraction pattern comprising a peak at about 10.93° 2θ. In some embodiments, the polymorph (e.g., a beta form) has an X-ray powder diffraction pattern comprising peaks at about 7.80° and 10.93° 2θ. In some embodiments, the polymorph (e.g., a beta form) has an X-ray powder diffraction pattern comprising peaks at about 7.80°, 9.79°, 10.93°, 11.95°, 16.95°, 17.58°, 18.94°, 20.80°, 21.95°, 24.02°, 25.17°, and 28.25° 2θ. In some embodiments, the polymorph (e.g., a beta form) has an X-ray powder diffraction pattern substantially as shown in FIG. 38.

In some embodiments, the polymorph (e.g., a gamma form) has an X-ray powder diffraction pattern comprising a peak at about 5.62° 2θ. In some embodiments, the polymorph (e.g., a gamma form) has an X-ray powder diffraction pattern comprising peaks at about 4.31° and 5.62° 2θ. In some embodiments, the polymorph (e.g., a gamma form) has an X-ray powder diffraction pattern comprising peaks at about 4.31°, 5.62°, and 6.71° 2θ. In some embodiments, the polymorph (e.g., a gamma form) has an X-ray powder diffraction pattern substantially as shown in FIG. 47.

In some embodiments, the polymorph (e.g., an alpha form) has a differential scanning calorimetry thermogram comprising an endothermic peak at about 157° C. In some embodiments, the polymorph (e.g., a beta form) has a differential scanning calorimetry thermogram comprising an endothermic peak at about 163° C. In some embodiments, the polymorph (e.g., an alpha and beta form) has a differential scanning calorimetry thermogram substantially as shown in FIG. 34.

In some embodiments, the polymorph (e.g., a beta form) has a thermogravimetric analysis and differential scanning calorimetry thermogram substantially as shown in FIG. 39.

In some embodiments, the polymorph has a solubility in water of about 5 μg/mL or about 6 μg/mL. In some embodiments, such solubility is at 37° C.

In some aspects, the present disclosure relates to an amorphous form of a compound of formula (I):

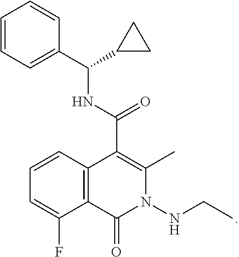 [SMILES: CCNn1c(C)c(C(=O)N[C@H](c2ccccc2)C2CC2)c2cccc(F)c2c1=O]

In some embodiments, the amorphous form has an X-ray powder diffraction pattern substantially as shown in FIG. 42. In some embodiments, the amorphous form has a thermogravimetric analysis and differential scanning calorimetry thermogram substantially as shown in FIG. 43. In some embodiments, the amorphous form has a solubility in water of 5 μg/mL. In some embodiments, such solubility is at 37° C.

In some aspects, the present disclosure relates to a beta form polymorph of a compound of formula (I):

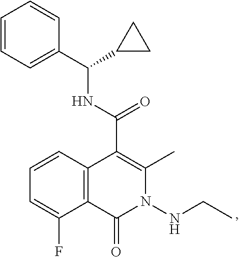 [SMILES: CCNn1c(C)c(C(=O)N[C@H](c2ccccc2)C2CC2)c2cccc(F)c2c1=O]

wherein the polymorph is substantially pure.

In some aspects, the present disclosure relates to an alpha form polymorph of a compound of formula (I):

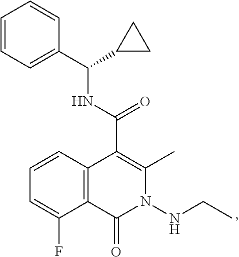 [SMILES: CCNn1c(C)c(C(=O)N[C@H](c2ccccc2)C2CC2)c2cccc(F)c2c1=O]

wherein the polymorph is substantially pure.

In some aspects, the present disclosure relates to a gamma form polymorph of a compound of formula (I):

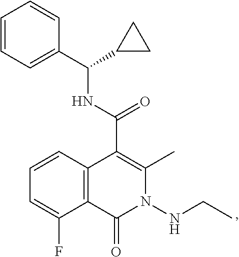 [SMILES: CCNn1c(C)c(C(=O)N[C@H](c2ccccc2)C2CC2)c2cccc(F)c2c1=O]

wherein the polymorph is substantially pure.

In some aspects, the present disclosure relates to an amorphous form of a compound of formula (I):

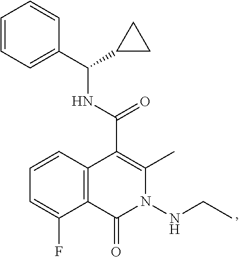 [SMILES: CCNn1c(C)c(C(=O)N[C@H](c2ccccc2)C2CC2)c2cccc(F)c2c1=O]

wherein the amorphous form is substantially pure.

In some aspects, the present disclosure relates to a pharmaceutical composition comprising the polymorph or amorphous form as described herein and a pharmaceutically acceptable carrier.

In some embodiments, the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

In some embodiments, the pharmaceutical composition is formulated for oral, intraarterial, intravenous or topical administration. In some embodiments, the pharmaceutical composition is formulated as a hard or soft capsule, a tablet, a syrup, a suspension, a solid dispersion, a wafer, or an elixir. In some embodiments, the pharmaceutical composition is formulated as a lotion, a cream, a gel, an oil, an ointment, a salve, or a suspension. In some embodiments, the pharmaceutical composition is formulated as a transdermal patch.

In some embodiments, the pharmaceutical composition described herein further includes an agent that enhances solubility and dispersibility.

In some aspects, the present disclosure relates to a pharmaceutical composition comprising a micronized beta form polymorph of a compound of formula (I), wherein the polymorph has a purity of greater than 99%, and wherein the pharmaceutical composition is formulated as a tablet.

In some aspects, the present disclosure relates to a method of treating a subject in need thereof, comprising administering to the subject a polymorph or amorphous form as described herein.

In some aspects, the present disclosure relates to a method of treating or preventing a condition or disease in a subject in need thereof, comprising administering to the subject the pharmaceutical composition as described herein.

In some embodiments, the disease is selected from psychosis; schizophrenia; schizophrenoform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; major depression; general anxiety disorder; bipolar disorder (maintenance treatment, recurrence prevention and stabilization); mania; hypomania; cognitive impairment; ADHD; obesity; appetite reduction; cognitive disorders; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; PTSD; dementia and agitation and delirium in the elderly; inflammatory diseases including irritable bowel syndrome and inflammatory bowel disorders; emesis; pre-eclampsia; airway hyperresponsiveness; reproduction disorders and sex hormone-dependent diseases including but not limited to benign prostatic hyperplasia (BPH), metastatic prostatic carninoma, testicular cancer, breast cancer, androgen dependent acne, male pattern baldness, endometriosis, abnormal puberty, uterine fibrosis, hormone-dependent cancers, hyperandrogenism, hirsutism, virilization, polycystic ovary syndrome (PCOS), HAIR-AN syndrome (hyperandrogenism, insulin resistance and acanthosis nigricans), ovarian hyperthecosis (HAIR-AN with hyperplasia of luteinized theca cells in ovarian stroma), other manifestations of high intraovarian androgen concentrations (e.g. follicular maturation arrest, atresia, anovulation, dysmenorrhea, dysfunctional uterine bleeding, infertility) and androgen-producing tumor (virilizing ovarian or adrenal tumor); and gynecological disorders and infertility.

In some embodiments, the disease is schizophrenia.

In some embodiments, the condition is hot flashes. In some embodiments, the hot flashes are associated with removal of ovaries or testes of the subject, breast cancer treatment, androgen deprivation therapy, hypogonadism or low serum gonadotropin levels, the subject having leukemia, non-dipper hypertension, carcinoid syndrome, post-menopausal hyperandrogenism, or precocious puberty.

In some embodiments, the disease or condition is excess body fat and/or excess body weight. In some embodiments, the disease or condition is a leptin-related disease. In some embodiments, the disease or condition is a hormonal imbalance.

In some aspects, the present disclosure relates to the use of a polymorph or amorphous form as described herein in the manufacture of a medicament for the treatment of a disease.

In some aspects, the present disclosure relates to a method of producing a polymorph (e.g., a beta form polymorph) of a compound of formula (I):

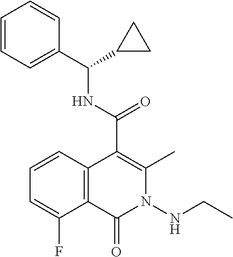 [SMILES: CCNn1c(C)c(C(=O)N[C@H](c2ccccc2)C2CC2)c2cccc(F)c2c1=O]

comprising combining a compound of formula (I) and isopropyl acetate to form a first solution; distilling the first solution to replace the isopropyl acetate with acetone and to form a second solution; adding n-heptane to the second solution to form a suspension, wherein the acetone:n-heptane ratio is 1:2; heating the suspension to a temperature of 55° C. and slowly cooling the heated suspension to a temperature of −20° C. to form a composition comprising a beta form polymorph of the compound of formula (I).

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 provides the chemical structure of SJB-01. pKa assignment of SJB-01 (L100142-1-1), experimentally determined pKa values are located near the corresponding acidic (red) or basic (blue) site with the theoretical pKa values shown in the bracket below.

FIG. 2 provides a Yasuda-Shedlovsky plot of SJB-01 used for extrapolation to 0% co-solvent (L100142-69-1).

FIG. 3 provides a graph showing distribution of the SJB-01 (L100142-69-1) species as a function of pH.

FIG. 4 provides Bjerrum plots of SJB-01 as a function of pH showing the distribution of experimental data (dots) fit with the theoretical (solid line) (L100142-69-1).

FIG. 5 provides Log D distribution profile as a function of pH (L100142-65-4), log _{D7.40} was 3.04.

FIG. 6 provides Bjerrum plots of SJB-01 as a function of pH showing the distribution of experimental data (dots) fit with the theoretical (solid line). The secondary solid line corresponds to the distribution of species in aqueous media in the absence of a partition solvent (L100142-65-4).

FIG. 7 shows XRPD pattern of SJB-01 (Lot#2223168, L100142-1-1), Pattern β.

FIG. 8 provides TGA/DSC thermograms of SJB-01 (Lot#2223168, L100142-1-1), Pattern β.

FIG. 9 provides DVS isotherms of SJB-01 (Lot#2223168, L100142-1-1), Pattern β. There is no change in form after the experiment.

FIG. 10 provides solution ¹H NMR spectrum of SJB-01 (Lot#2223168, L100142-1-1), Pattern β, in DMSO-d₆.

FIG. 11 provides microscope image of SJB-01 (Lot#2223168, L100142-1-1), Pattern β.

FIG. 12 shows particle size distribution of SJB-01 (Lot#2223168, L100142-1-1), Pattern β.

FIG. 13 provides chromatograms for L100142-21-1 (lot#17-12607), L100142-1-1 (lot#2223168) and a blank (diluent, ACN:water 1:1 vol.) in ascending order. Peak table is presented in Table 1.

FIG. 14 shows XRPD pattern of Pattern β (L100142-1-1) for reference and of solids from evaporation of IPA (L100142-5-17), IPA:water (L100142-5-23), MIBK (L100142-5-24 (α)) and MtBE (L100142-5-25) showing varying amounts of Pattern α, with L100142-5-24 being pure pattern α.

FIG. 15 shows XRPD patterns of Patterns α and β for reference, and new Pattern γ (+(3) exhibited by solids from recrystallization in EtOAc at −20° C. (L100142-16-2).

FIG. 16 shows XRPD patterns for solids from evaporation of IPA at 50° C. (L100142-28-3) (1) first sample, (2) same sample after 1 day, (3) the solids from the vial the next day, (4) Pattern β, exhibited by solids recovered from evaporation of MIBK at 50° C.

FIG. 17 provides DSC thermogram of solids from stagnant evaporation of IPA at 50° C., Pattern α (L100142-28-3).

FIG. 18 shows XRPD pattern of the solids from 1-PrOH when first sampled, and from the vial after sampling (L100142-37-1).

FIG. 19 shows XRPD pattern of the solids formed by seeding of the gel with L100142-37-1, resulting in Pattern β, and the solid obtained from unseeded gel, resulting in Pattern α with trace β.

FIG. 20 shows XRPD pattern of the solids from 1-PrOH when first sampled, after drying at 50° C. for 30 min, and from the vial 1 hr after sampling (L100142-37-6).

FIG. 21 provides DSC thermogram of dried Pattern α (L100142-37-6), still showing two melting endotherms characteristic of both Pattern α and β.

FIG. 22 shows XRPD pattern of the solids from 1-PrOH when first sampled and from the vial 1 hr after sampling (L100142-37-5).

FIG. 23 provides DSC thermogram for freebase Pattern β (L100142-1-1) first heated to 180° C., then cooled to 25° C. The pan contents were analyzed by XRPD after the experiment (FIG. 24).

FIG. 24 shows XRPD of SJB-01 Pattern β (L100142-1-1) for reference and the amorphous pattern exhibited by material that had been melted then cooled, observed by DSC (thermogram in FIG. 23).

FIG. 25 shows XRPD pattern of the amorphous powder (L100142-19-1) showing low intensity, broad lumps.

FIG. 26 provides TGA/DSC thermograms of free-flowing amorphous SJB-01 (L100142-19-1).

FIG. 27 provides TGA/DSC thermogram of free-flowing amorphous freebase (L100142-19-1) heated to 140° C. The post-experiment pan contents were analyzed by XRPD (FIG. 28).

FIG. 28 shows XRPD patterns of Pattern α and β for reference, and of solids recovered from heating amorphous freebase to 140° C. immediately after heat treatment, then two weeks later. The heat treatment was observed by TGA/DSC, thermograms for which are presented in FIG. 27.

FIG. 29 shows XRPD patterns of powdered amorphous freebase (L100142-19-1) after formation (1) (two patterns are presented since two different XRPD samples were prepared from the same batch at that time that look marginally different), after 5 days (2), after 17 days (3), and after heating to 85° C. (4) (thermogram of heating presented in FIG. 30).

FIG. 30 provides DSC thermogram of the heating of powdered amorphous freebase (L100142-19-1) to 85° C. XRPD of the pan contents after the experiment exhibit the pattern labeled (4) in FIG. 29.

FIG. 31 shows transformation of Pattern α+β that was produced by heating powdered amorphous freebase (100142-19-1) to 140° C. to a pattern that only has traces of Pattern α after sitting at room temperature for 2 weeks. Patterns β and α are shown for reference.

FIG. 32 shows XRPD patterns of amorphous SJB-01 (L100142-19-1) and Pattern α (L100142-37-5) before and after 1 week at 40° C./75% RH. Pattern β is shown for reference.

FIG. 33 shows XRPD pattern of Pattern α (sample L100142-37-1a).

FIG. 34 provides DSC thermogram of dried Pattern α (L100142-37-6), showing two melting endotherms characteristic of both Pattern α and β. There is a possibility that Pattern α recrystallizes as Pattern β upon melting followed by melting of Pattern β.

FIG. 35 provides DVS isotherm for Pattern α (L100142-37-5).

FIG. 36 shows XRPD patterns for Pattern α (L100142-37-5) before (bottom) and after (top) DVS.

FIG. 37 provides microscope image of Pattern α (L100142-37-6).

FIG. 38 shows XRPD pattern of solid SJB-01 (Lot#2223168, L100142-1-1), Pattern β.

FIG. 39 provides TGA/DSC thermograms of solid SJB-01 (Lot#2223168, L100142-1-1), Pattern β.

FIG. 40 provides DVS isotherms of solid SJB-01 (Lot#2223168, L100142-1-1), Pattern β. There is no change in form after the experiment.

FIG. 41 provides microscope image of solid SJB-01 (Lot#2223168, L100142-1-1), Pattern β.

FIG. 42 shows XRPD pattern of amorphous SJB-01 solid.

FIG. 43 provides TGA/DSC thermograms of amorphous SJB-01 solid.

FIG. 44 provides microscope image of free-flowing amorphous freebase SJB-01 (L100142-19-1).

FIG. 45 provides DVS isotherm for powdered amorphous freebase SJB-01 (L100142-34-1). Post-experiment XRPD is presented in FIG. 46.

FIG. 46 shows XRPD pattern for the amorphous freebase SJB-01 before and after DVS (isotherm presented in FIG. 45).

FIG. 47 shows XRPD of SJB-01 Pattern γ by subtraction from Pattern 13.

FIG. 48 shows all XRPD of crystalline solids observed in the salt screening showing Pattern β. Patterns correspond to data in Table 6.

FIG. 49 shows XRPD patterns, all matching Pattern β, for solids slurried in various solvents at room temperature (22-23° C.). Conditions corresponding to ID labels are found in Table 7.

FIG. 50 shows XRPD patterns, all matching Pattern β, for solids slurried in various solvents at 50° C. Conditions corresponding to ID labels are found in Table 7.

FIG. 51 shows XRPD patterns from slow and fast cooling crystallizations with SJB-01 (L100142-1-1) as the input material, all match Pattern β. Experimental details and results are summarized in Table 8.

FIG. 52 shows XRPD patterns from slow cooling crystallizations with SJB-01 (L100142-21-1) as the input material, all match Pattern β. Labels correspond to Table 9.

FIG. 53 shows XRPD patterns from fast cooling crystallizations with SJB-01 (L100142-21-1) as the input material, all match Pattern β. Labels correspond to Table 9.

FIG. 54 shows XRPD patterns of solids obtained from stagnant cooling crystallization experiments. Labels correspond to experiment IDs in Table 10.

FIG. 55 shows XRPD patterns of solids obtained from anti-solvent crystallization experiments, all match Pattern β. Labels correspond to experiment IDs in Table 11.

FIG. 56 shows XRPD patterns of freebase Pattern β (L100142-1-1) for reference, and solids obtained from flash evaporation of THF (L100142-13-3), acetone (L100142-13-4), and MIBK (L100142-13-6). Experiment details are presented in Table 12.

FIG. 57 shows XRPD pattern of solids from rotary evaporation of MIBK (L100142-25-1) matching Pattern β.

FIG. 58 shows XRPD patterns of solids obtained from stirred evaporation of MIBK or IPA, all match Pattern β. Labels correspond to experiment IDs in Table 14.

FIG. 59 shows XRPD patterns of solids obtained from evaporation of IPA at 40, 50 and 60° C. All are Pattern β.

FIG. 60 shows XRPD patterns of solids recovered after milling SJB-01 (L100142-1-1, Pattern β) dry or with 15 μL of a chosen solvent. There are no changes in form, but the dry and MtBE drop milled samples are substantially less crystalline than the starting material. Labels correspond to experiment IDs in Table 16.

FIG. 61 shows XRPD patterns of solids obtained from the in-situ salt formation and disproportionation with water (ordered L100142-18-1→L100142-18-12 from bottom to top), details for which can be found in Table 17. The patterns exhibited by all solids are Pattern β.

FIG. 62 provides photographs of clear, “cracked-glass” freebase obtained after cooling/solidification of the melt (left) and of white, powdery, amorphous freebase after scraping/crushing the glass with a needle and spatula (right).

FIG. 63 provides DSC thermogram with time and temperature on the x-axis for powdered amorphous freebase (L100142-19-1) to illustrate the thermal events in chronological order as well as highlight differences in behavior in the same thermal range from one cycle to the next.

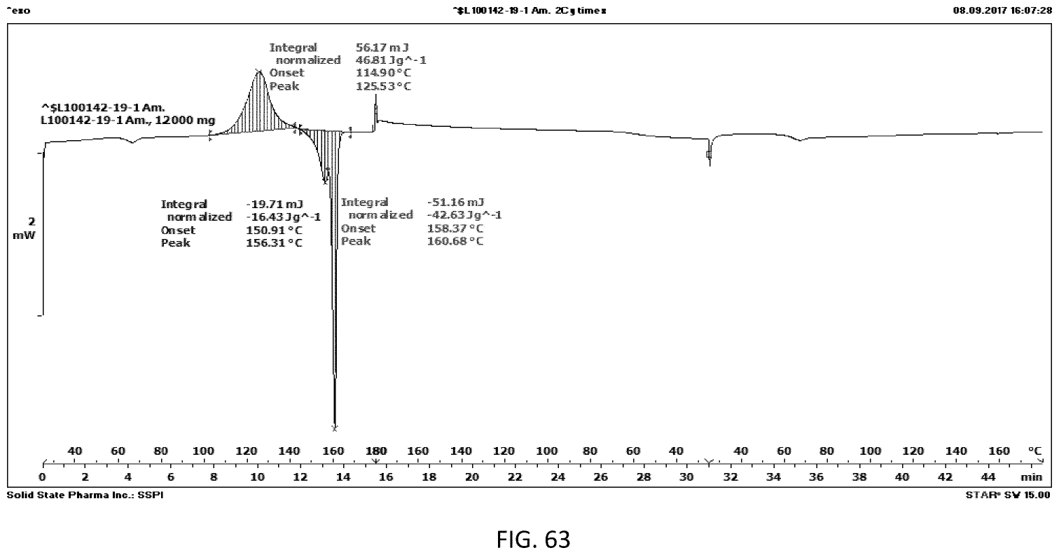

FIG. 64 provides DSC thermogram with temperature on the x-axis for powdered amorphous freebase (L100142-19-1) to illustrate the thermal events in chronological order as well as highlight differences in behavior in the same thermal range from one cycle to the next.

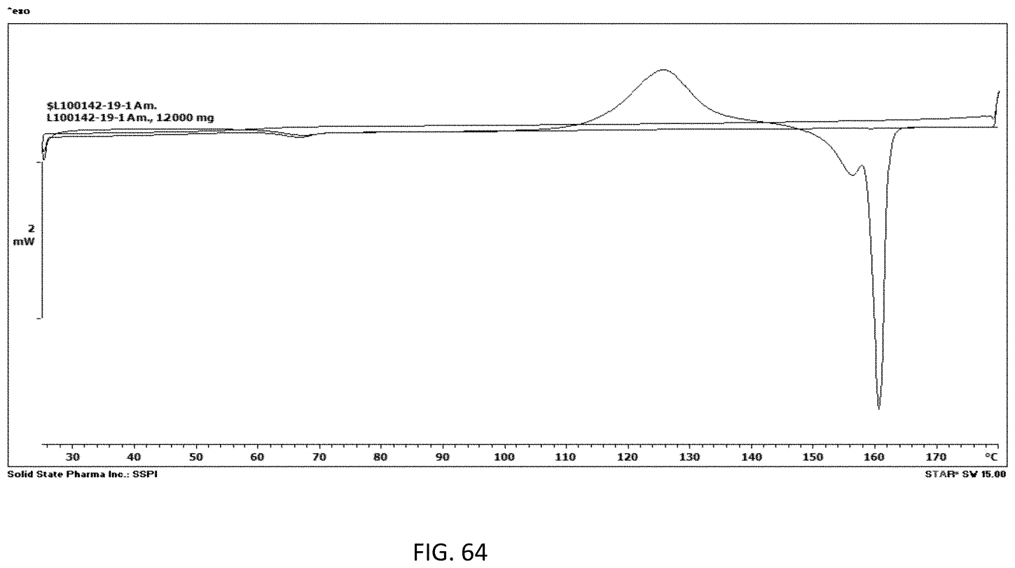

FIG. 65 provides DSC thermogram recorded for the heating of the sample (in the same pan) from the DSC experiment in FIGS. 63-64 after poking with a needle.

FIG. 66 shows XRPD patterns of freebase Pattern β (L100142-1-1) for reference, and solids obtained from slurry of amorphous freebase in IPA (L100142-14-1), water (L100142-14-2), IPA:water (9:1 vol.) (L100142-14-3), IPAc (L100142-14-4), MtBE (L100142-14-5), and cyclohexane (L100142-14-6). Experiment details are presented in Table 18.

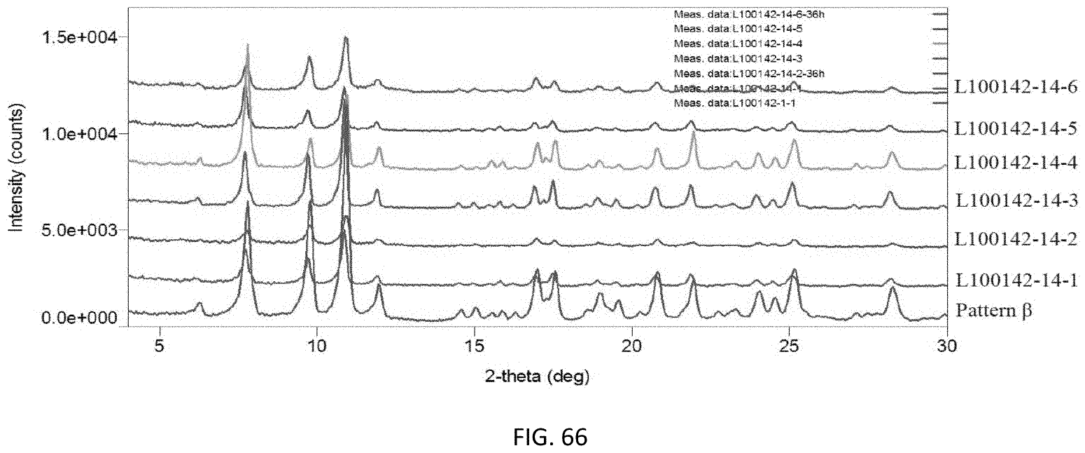

FIG. 67 shows intrinsic dissolution rate of SJB-01 in 1 mM HCL, pH 3.0.

FIGS. 68A-68B show absorption of solid compound in rats. FIG. 68A shows absorption of solid micronized SJB-01 in Sprague Dawley rats. FIG. 68B shows dose proportionality of the compound.

FIG. 69 provides a DSC thermogram of SJB-01, alpha form.

FIG. 70 provides a DSC thermogram of SJB-01, beta form.

FIG. 71 provides a TGA thermogram of SJB-01, alpha form.

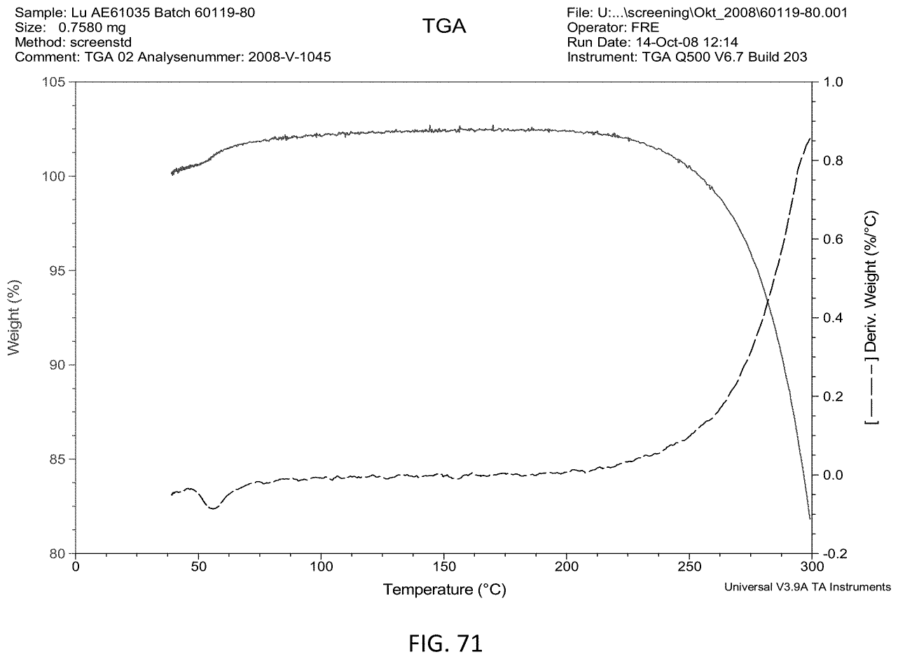

FIG. 72 provides HSM polarization pictures of SJB-01. (Note: *Needle formed crystals are also observed at this temperature).

FIG. 73 provides x-ray diffractograms of SJB-01. Red: alpha (batch 60119-80), blue: beta (batch 60095-076).

FIG. 74 provides IR spectrum of SJB-01, alpha.

FIG. 75 provides IR spectrum of SJB-01, alpha (red) and beta (blue).

FIG. 76 provides UV spectrum of SJB-01.

FIG. 77 provides XRPD patterns of SJB-01 (1) calculated from the single crystal data collected at 100 K, (2) calculated from the single crystal data collected at 290 K, and (3) Pattern β collected from a bulk sample at room temperature (ca. 298 K).

FIG. 78 provides a thermal ellipsoid representation at the 50% probability level for the two crystallographically independent molecules of SJB-01 (L100149-74-13) with atomic labelling scheme. Dashed lines indicate close-contacts. Hydrogen bonds drawn as thin dashed lines. Note that the N4-H4 . . . F1 hydrogen bond is quite long (2.556(19) Å) and, therefore, at the most a very weak interaction. The two molecules are shown in their correct relative orientations.

FIG. 79 provides an overlay of the two independent molecules in SJB-01 (L100149-74-13) calculated through all labelled atoms after inverting the second molecule. RMS deviation is 0.09 Å.

FIG. 80 provides packing plots of the structure of SJB-01 (L100149-74-13). Panels A, B, and C show the packing in projections along the crystallographic a-, b-, and c-axes, respectively. Hydrogen bonds are drawn as thin dashed lines.

FIG. 81 provides a simulated powder diffractogram for the structure of SJB-01 (L100149-74-13).

FIG. 82 provides a plot of SJB-01 solubility (scale 0.004-0.011 mg/mL) vs. pH (scale 0-8) of buffered solutions.

FIG. 83 provides a calibration curve used for relating the HPLC peak area to concentration of SJB-01.

### DETAILED DESCRIPTION OF THE INVENTION

The disclosure relates to the discovery of multiple polymorphic and amorphous forms 2-Ethylamino-8-fluoro-3-methyl-1-oxo-1,2-dihydro-isoquinoline-4-carboxylic acid ((S)-cyclopropyl-phenyl-methyl)-amide (also referred to herein as SJB-01). SJB-01 is an NK3 receptor antagonist. SJB-01 is a selective NK3 receptor antagonist (e.g., selective for NK3 over, at least, NK1 and NK2). In some embodiments the disclosure relates to polymorphic and amorphous forms of compounds of formula (I):

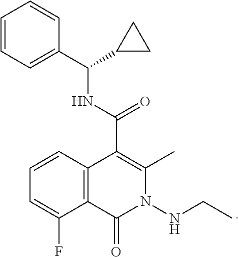 [SMILES: CCNn1c(C)c(C(=O)N[C@H](c2ccccc2)C2CC2)c2cccc(F)c2c1=O]

Also disclosed are pharmaceutical compositions comprising the described polymorphic and amorphous compounds, methods of treating conditions or diseases, such as psychosis or hot flashes, using compositions comprising the described compounds, and methods of producing the described compounds.

Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, kits and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, kits and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, pharmaceutically acceptable salts include pharmaceutical acceptable acid addition salts, pharmaceutically acceptable metal salts, ammonium and alkylated ammonium salts. Acid addition salts include salts of inorganic acids as well as organic acids.

Examples of suitable inorganic acids include hydrochloric, hydrobromic, hydroiodic, phosphoric, sulfuric, sulfamic, nitric acids and the like. In certain embodiments the pharmaceutically acceptable salt is a hydrochloride salt.

Examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid addition salts include the pharmaceutically acceptable salts listed in J. Pharm. Sci. 1977,66,2, which is incorporated herein by reference.

Examples of metal salts include lithium, sodium, potassium, magnesium salts and the like.

Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

As used herein, the term “therapeutically effective amount” of a compound means an amount sufficient to cure, alleviate or partially arrest the clinical manifestations of a given disease and its complications in a therapeutic intervention comprising the administration of said compound. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

As used herein, the term “treatment” and “treating” means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relieve the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatments are two separate aspects of the invention. The patient to be treated is preferably a mammal, in particular a human being.

Terms and Abbreviations

XRPD X-ray powder diffraction;

DSC Differential scanning calorimetry

TGA Thermogravimetric analysis

DVS Dynamic vapor sorption

HPLC High pressure liquid chromatography

KF Karl Fisher titration

NMR Nuclear magnetic resonance

HSM Hot stage microscopy

PLM Polarized light microscopy

IR Infrared

UV-vis Ultraviolet-visible

1-PrOH 1-proponal

IPA 2-proponal

ACN Acetonitrile

BA Benzyl alcohol

DCM Dichloromethane

DMSO Dimethyl sulfoxide

EtOH Ethanol

EtOAc Ethyl acetate

IPAc Isopropyl acetate

MeOH Methanol

MeOAc Methyl acetate

MBK Methyl butyl ketone

MEK Methyl ethyl ketone

MIBK Methyl isobutyl ketone

DMAc N,N-dimethylacetamide

DMF N,N-dimethylformamide

NMP N-methyl pyrrolidone

MtBE tert-Butyl methyl ether

THF Tetrahydrofuran

TFA Trifuoroacetic acid

TFE Trifluoroethanol

Polymorphic and Amorphous Forms

The disclosure relates to the discovery of multiple polymorphic and amorphous forms of 2-ethylamino-8-fluoro-3-methyl-1-oxo-1,2-dihydro-isoquinoline-4-carboxylic acid ((S)-cyclopropyl-phenyl-methyl)-amide (also referred to herein as SJB-01), having a molecular structure of formula (I):

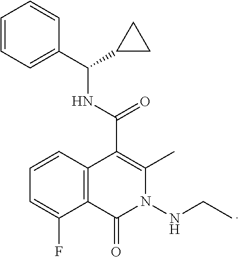 [SMILES: CCNn1c(C)c(C(=O)N[C@H](c2ccccc2)C2CC2)c2cccc(F)c2c1=O]

SJB-01 has a molecular formula of C₂₃H₂₄FN₃O₂ and a molecular weight of 393.46 g/mol. In some embodiments SJB-01 is characterized as having two pKa values, an acidic value of 5.83±0.11 and a basic value of 3.03±0.11. In some embodiments SJB-01 is further characterized as having a Log P value of 4.73±0.14 and a Log D profile where log D_{7.40}=3.04.

In some embodiments the multiple polymorphic forms include an alpha form, a beta form, and a gamma form. In some aspects provided is an alpha polymorphic form of 2-ethylamino-8-fluoro-3-methyl-1-oxo-1,2-dihydro-isoquinoline-4-carboxylic acid ((S)-cyclopropyl-phenyl-methyl)-amide. In other aspects provided is a beta polymorphic form of 2-ethylamino-8-fluoro-3-methyl-1-oxo-1,2-dihydro-isoquinoline-4-carboxylic acid ((S)-cyclopropyl-phenyl-methyl)-amide. In some aspects provided is a gamma polymorphic form of 2-ethylamino-8-fluoro-3-methyl-1-oxo-1,2-dihydro-isoquinoline-4-carboxylic acid ((S)-cyclopropyl-phenyl-methyl)-amide. In other aspects provided is an amorphous form of 2-ethylamino-8-fluoro-3-methyl-1-oxo-1,2-dihydro-isoquinoline-4-carboxylic acid ((S)-cyclopropyl-phenyl-methyl)-amide.

Alpha Form

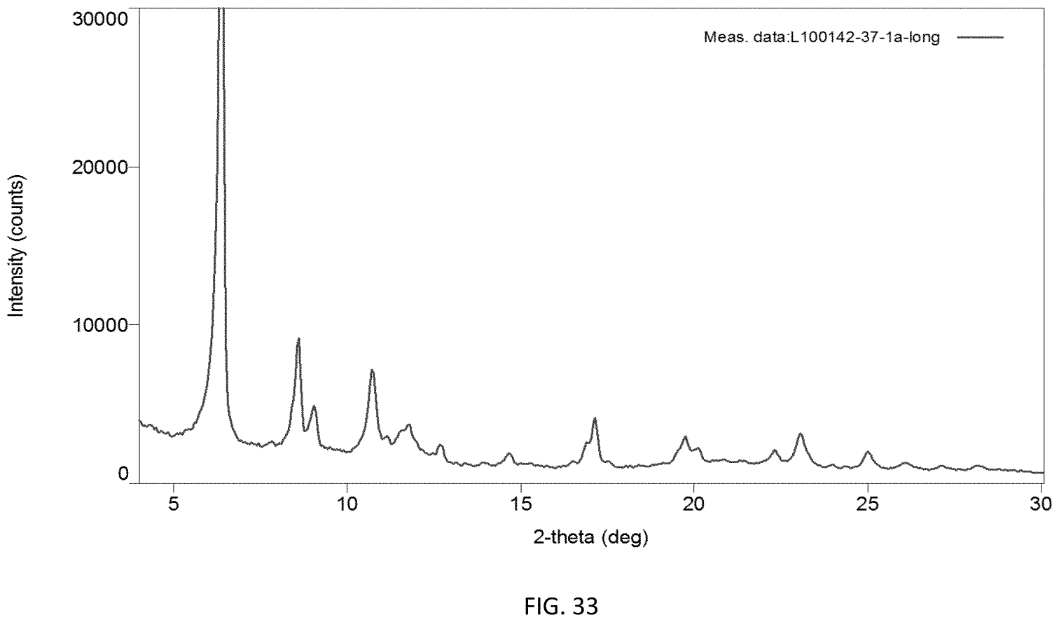

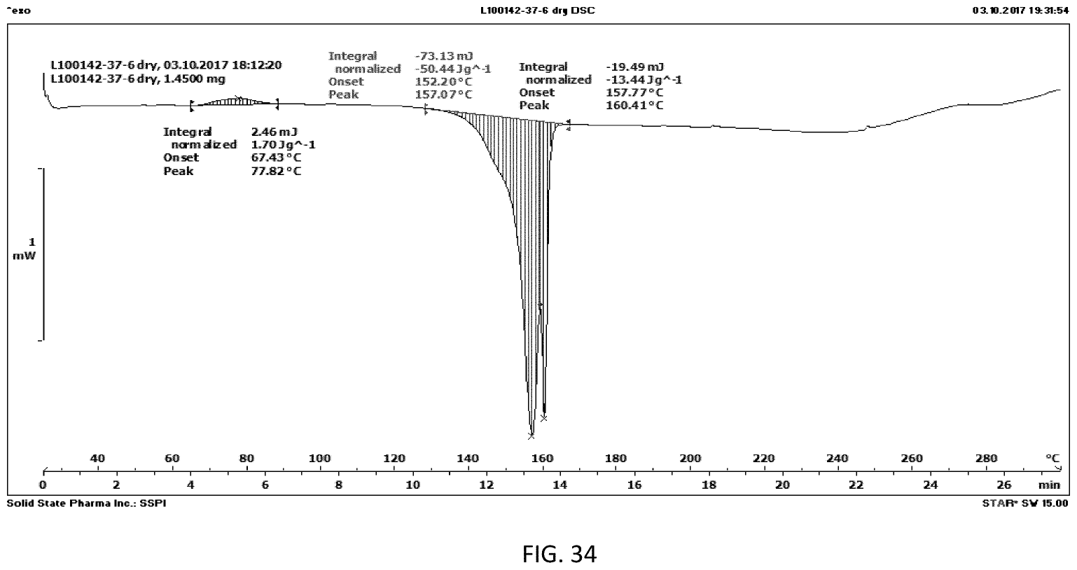

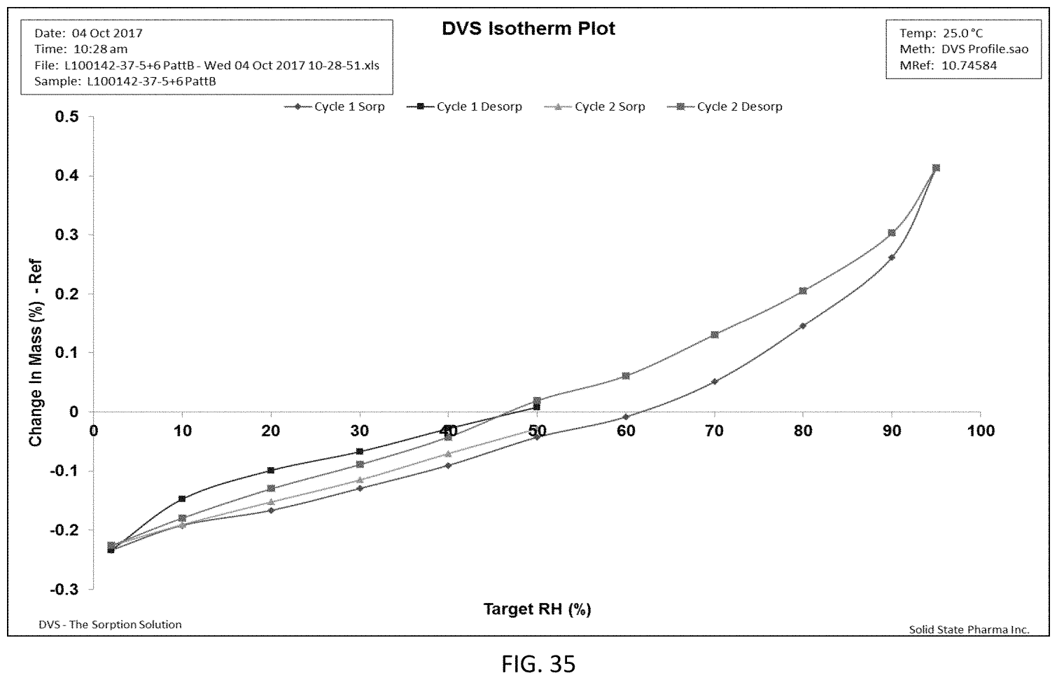

In some embodiments, a polymorph of a compound of formula (I) is an alpha form. In some aspects the polymorph exhibits an X-ray powder diffraction (XRPD) pattern substantially as shown in FIG. 33. In some aspects the polymorph exhibits a differential scanning calorimetry (DSC) thermogram substantially as shown in FIG. 34. The polymorph may exhibit a dynamic vapor sorption (DVS) isotherm plot substantially as shown in FIG. 35.

The term “substantially as shown in” when referring, for example, to an XRPD pattern, a DSC thermogram, DVS plot, or a TGA graph includes a pattern, thermogram, plot, or graph that is not necessarily identical to those depicted herein, but that falls within the limits of experimental error or deviations when considered by one of ordinary skill in the art.

In some aspects the polymorph of a compound of formula (I) has at least one, at least two, or at least three of the following: (a) an XRPD pattern substantially as shown in FIG. 33, (b) a DSC thermogram substantially as shown in FIG. 34, and (c) a DVS isotherm plot substantially as shown in FIG. 35.

In some aspects a polymorph of the compound of formula (I) (e.g., an alpha form polymorph) has an XRPD pattern displaying at least two, at least three, at least four, at least five, or at least six of the degree 2θ-reflections with the greatest intensity as the XRPD pattern substantially as shown in FIG. 33. It should be understood that relative intensities can vary depending on a number of factors, including sample preparation, mounting, and the instrument and analytical procedure and settings used to obtain the spectrum. The peak assignments listed herein are intended to encompass variations of +/−0.2 degrees 2θ.

In certain aspects a polymorph of the compound of formula (I) has an XRPD pattern comprising a peak at about 6.37° 2θ (+/−0.2 degrees 2θ). In some aspects a polymorph of the compound of formula (I) has an XRPD pattern comprising peaks at about 6.37° and 8.60° 2θ (+/−0.2 degrees 2θ). In some aspects a polymorph of the compound of formula (I) has an XRPD pattern comprising peaks at about 6.37°, 8.60°, and 10.72° 2θ (+/−0.2 degrees 2θ). In some aspects a polymorph of the compound of formula (I) has an XRPD pattern comprising peaks at about 6.37°, 8.60°, 9.06°, 10.72°, and 14.62° 2θ (+/−0.2 degrees 2θ). In some aspects a polymorph of the compound of formula (I) has an XRPD pattern comprising peaks at about 6.37°, 8.60°, 9.06°, 10.72°, 11.76°, 17.13°, and 23.02° 2θ (+/−0.2 degrees 2θ). In some aspects a polymorph of the compound of formula (I) has an XRPD pattern comprising peaks at about 6.37°, 8.60°, 9.06°, 10.72°, 11.76°, 12.71°, 14.62°, 17.13°, 19.74°, 23.02°, and 24.99° 2θ (+/−0.2 degrees 2θ).

In some aspects, a polymorph of the compound of formula (I) (e.g., an alpha form) has an XRPD pattern comprising peaks at the interplanar spacing (d spacing) values of about 13.86, 10.27, and 8.25 (Å). In certain such aspects, the XRPD pattern further comprises peaks at the interplanar spacing (d spacing) values of about 9.75, 7.52, 5.17, and 3.86 (Å).

In some aspects, a polymorph of the compound of formula (I) (e.g., an alpha form) is characterized by an XRPD pattern substantially similar to that set forth in FIG. 33.

In some aspects a polymorph of the compound of formula (I) (e.g., an alpha form polymorph) has a DSC thermogram comprising an endothermic peak at about 157.1° C. In some aspects a polymorph of the compound of formula (I) has a solubility in water and simulated fluids in the range of 6-8 μg/mL at about 37° C. In certain aspects a polymorph of the compound of formula (I) has a solubility in water of 6 μg/mL at about 37° C. In certain aspects a polymorph of the compound of formula (I) has a solubility in fasted state simulated intestinal fluid (FaSSIF) of 8 μg/mL at about 37° C. In certain aspects a polymorph of the compound of formula (I) has a solubility in fasted state simulated gastric fluid (FaSSGF) of 7 μg/mL at about 37° C.

In some embodiments, a polymorph of compound (I) is a substantially pure alpha form polymorph. In some aspects, “substantially pure” refers to a substance free of other substances, including other polymorphic forms, amorphous forms, and/or impurities. In some aspects, the compound of formula (I) is an alpha form and has a purity of greater than 80%, e.g., greater than 85%, greater than 90%, greater than 92.5%, greater than 95%, greater than 96%, greater than 97%, greater than 98%, greater than 99%, greater than 99.5%, greater than 99.8% (and in certain embodiments of any of the foregoing, less than 100%). In some aspects, the compound of formula (I) is an alpha form having a purity of about 95% or at least about 95%.

Beta Form

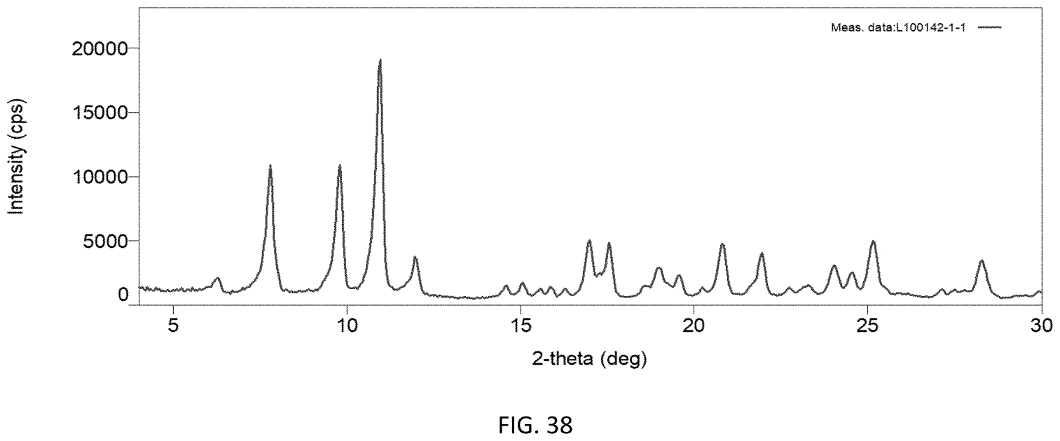

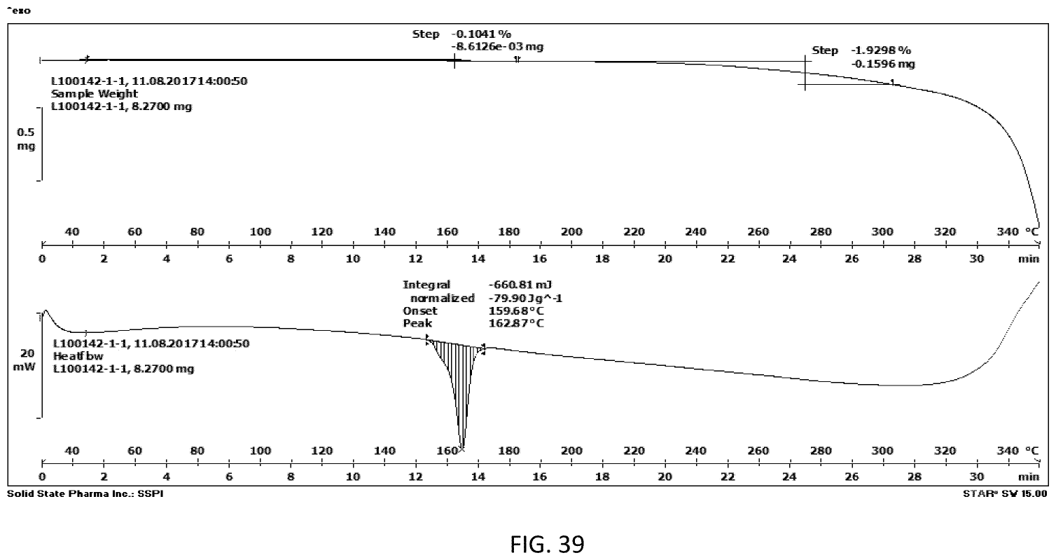

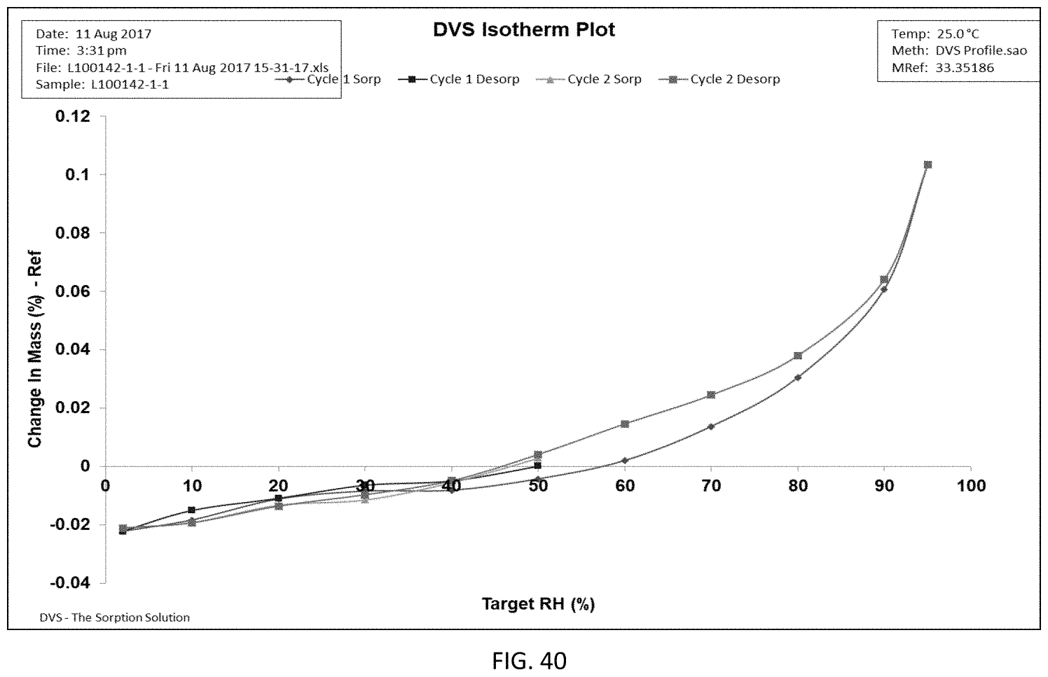

In some embodiments, a polymorph of a compound of formula (I) is a beta form. In some aspects the polymorph exhibits an X-ray powder diffraction (XRPD) pattern substantially as shown in FIG. 38. In some aspects the polymorph exhibits a thermographic analysis (TGA)/differential scanning calorimetry (DSC) thermogram substantially as shown in FIG. 39. The polymorph may exhibit a dynamic vapor sorption (DVS) isotherm plot substantially as shown in FIG. 40.

In some aspects the polymorph of a compound of formula (I) has at least one, at least two, or at least three of the following: (a) an XRPD pattern substantially as shown in FIG. 38, (b) a TGA/DSC thermogram substantially as shown in FIG. 39, and (c) a DVS isotherm plot substantially as shown in FIG. 40.

In some aspects a polymorph of the compound of formula (I) (e.g., a beta form polymorph) has an XRPD pattern displaying at least two, at least three, at least four, at least five, or at least six of the degree 2θ-reflections with the greatest intensity as the XRPD pattern substantially as shown in FIG. 38. The peak assignments listed herein are intended to encompass variations of +/−0.2 degrees 2θ.

In certain aspects a polymorph of the compound of formula (I) has an XRPD pattern comprising a peak at about 10.93° 2θ (+/−0.2 degrees 2θ). In some aspects a polymorph of the compound of formula (I) has an XRPD pattern comprising peaks at about 7.80° and 10.93° 2θ (+/−0.2 degrees 2θ). In some aspects a polymorph has an XRPD pattern comprising peaks at about 7.80°, 9.79° and 10.93° 2θ (+/−0.2 degrees 2θ). In some aspects a polymorph of the compound of formula (I) has an XRPD pattern comprising peaks at about 7.80°, 9.79°, 10.93°, 25.17°, and 20.80° 2θ (+/−0.2 degrees 2θ). In some aspects a polymorph of the compound of formula (I) has an XRPD pattern comprising peaks at about 7.80°, 9.79°, 10.93°, 11.95°, 16.95°, 17.58°, 25.17°, and 20.80° 2θ (+/−0.2 degrees 2θ). In some aspects a polymorph of the compound of formula (I) has an XRPD pattern comprising peaks at about 7.80, 9.79°, 10.93°, 11.95°, 16.95°, 17.58°, 25.17°, 20.80°, 21.95°, and 28.25° 2θ (+/−0.2 degrees 2θ). In some aspects a polymorph of the compound of formula (I) has an XRPD pattern comprising peaks at about 7.80°, 9.79°, 10.93°, 11.95°, 16.95°, 17.58°, 18.94°, 20.80°, 21.95°, 24.02°, 25.17°, and 28.25° 2θ (+/−0.2 degrees 2θ).

In some embodiments, a polymorph (e.g., a beta form) of the compound of formula (I) has an XRPD pattern comprising peaks at the interplanar spacing (d spacing) values of about 11.323 and 8.086 (Å). In some aspects, a polymorph (e.g., a beta form) of the compound of formula (I) has an XRPD pattern comprising peaks at the interplanar spacing (d spacing) values of about 11.323, 9.029, and 8.086 (Å). In certain such aspects, the XRPD pattern further comprises peaks at the interplanar spacing (d spacing) values of about 7.40, 5.23, 5.04, 4.68, 4.53, 4.27, 4.05, 3.70, 3.62, 3.53, and 3.16 (Å).

In some aspects, a polymorph of the compound of formula (I) (e.g., a beta form) is characterized by an XRPD pattern substantially similar to that set for in FIG. 38.

In some aspects a polymorph of the compound of formula (I) (e.g., a beta form polymorph) has a DSC thermogram comprising an endothermic peak at about 163° C. In some aspects a polymorph of the compound of formula (I) has a solubility in water and simulated fluids in the range of 5-8 μg/mL or in the range of 6-10 μg/mL at about 37° C. Solubility of the polymorph may vary depending on the pH of the aqueous solution. In certain aspects a polymorph of the compound of formula (I) has a solubility in water of 5 μg/mL at about 37° C. In certain aspects a polymorph of the compound of formula (I) has a solubility in fasted state simulated intestinal fluid (FaSSIF) of 8 μg/mL at about 37° C. In certain aspects a polymorph of the compound of formula (I) has a solubility in fasted state simulated gastric fluid (FaSSGF) of 6 μg/mL at about 37° C. In some aspects a polymorph of the compound of formula (I) (e.g., a beta form) has a solubility in buffered solution of 6.4-9.8 μg/mL at about 37° C. In certain aspects the solubility of a polymorph of the compound of formula (I) (e.g., a beta form) in buffered solution decreases with an increase in pH.

In some aspects a polymorph of the compound of formula (I) (e.g., a beta form polymorph) has a morphology that is rod-like as identified using optical microscopy. In some aspects particles have a length of over 100 μm. In some aspects particles have a length of about 100 μm or longer.

In some embodiments, a polymorph of compound (I) is a substantially pure beta form polymorph. In some aspects, “substantially pure” refers to a substance free of other substances, including other polymorphic forms, amorphous forms, and/or impurities. Purity may be assessed by, for example, HPLC or NMR. In some aspects, the compound of formula (I) is a beta form and has a purity of greater than 80%, e.g., greater than 85%, greater than 90%, greater than 92.5%, greater than 95%, greater than 96%, greater than 97%, greater than 98%, greater than 99%, greater than 99.5%, greater than 99.8% (and in certain embodiments of any of the foregoing, less than 100%). In some aspects, the compound of formula (I) is a beta form having a purity of about 95% or at least about 95%. In some aspects, the compound of formula (I) is a beta form having a purity of about 99% or at least about 99%.

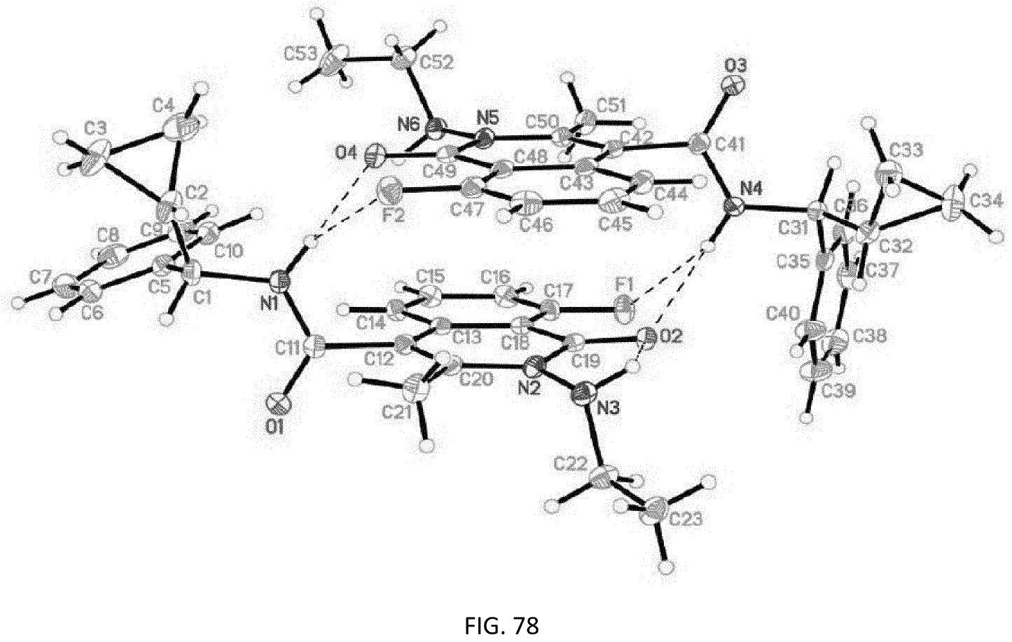

In some aspects, a polymorph of the compound of formula (I) (e.g., a beta form) has a crystal structure comprising two crystallographically independent molecules. In some aspects the two crystallographically independent molecules form a pseudo-centrosymmetric dimer. In some embodiments, a polymorph of the compound of formula (I) (e.g., a beta form) is characterized by a crystal structure comprising two crystallographically independent molecules substantially similar to that set for in FIG. 78.

Gamma Form

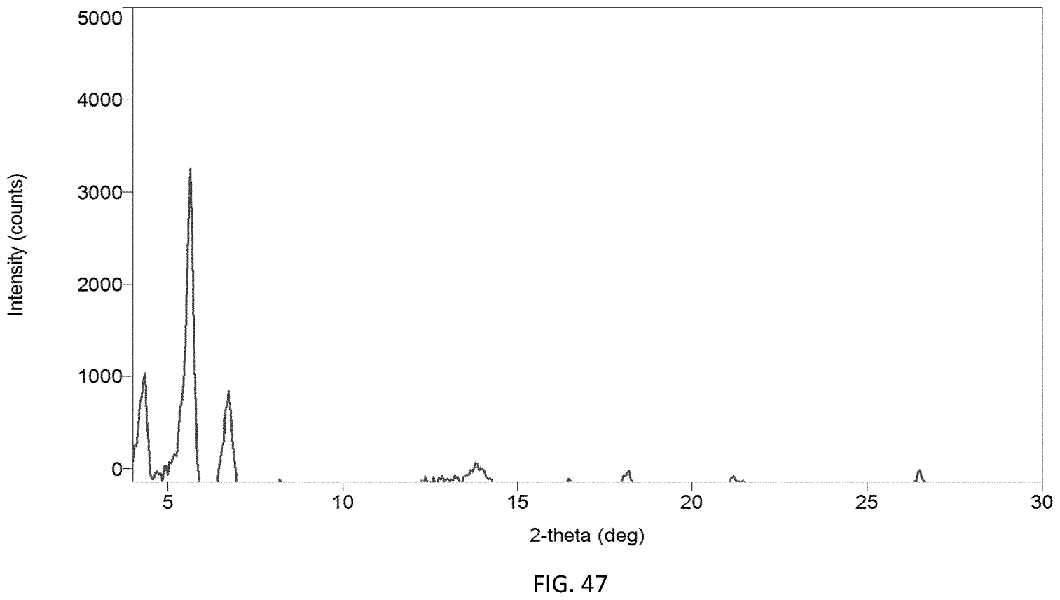

In some embodiments, a polymorph of a compound of formula (I) is a gamma form. In some aspects the polymorph exhibits an X-ray powder diffraction (XRPD) pattern substantially as shown in FIG. 47. In certain such embodiments, the gamma form is co-crystalized with the beta form.

In some aspects a polymorph of the compound of formula (I) (e.g., a gamma form polymorph) has an XRPD pattern displaying at least two, or at least three of the degree 2θ-reflections with the greatest intensity as the XRPD pattern substantially as shown in FIG. 47. The peak assignments listed herein are intended to encompass variations of +/−0.2 degrees 2θ.

In certain aspects a polymorph of the compound of formula (I) has an XRPD pattern comprising a peak at about 5.62° 2θ (+/−0.2 degrees 2θ). In some aspects a polymorph of the compound of formula (I) has an XRPD pattern comprising peaks at about 4.31° and 5.62° 2θ (+/−0.2 degrees 2θ). In some aspects a polymorph of the compound of formula (I) has an XRPD pattern comprising peaks at about 4.31°, 5.62°, and 6.71° 2θ (+/−0.2 degrees 2θ).

In some embodiments, a polymorph of compound (I) is a substantially pure gamma form polymorph. In some aspects, “substantially pure” refers to a substance free of other substances, including other polymorphic forms, amorphous forms, and/or impurities. In some aspects, the compound of formula (I) is a gamma form and has a purity of greater than 80%, e.g., greater than 85%, greater than 90%, greater than 92.5%, greater than 95%, greater than 96%, greater than 97%, greater than 98%, greater than 99%, greater than 99.5%, greater than 99.8% (and in certain embodiments of any of the foregoing, less than 100%). In some aspects, the compound of formula (I) is a gamma form having a purity of about 95% or at least about 95%.

Amorphous Form

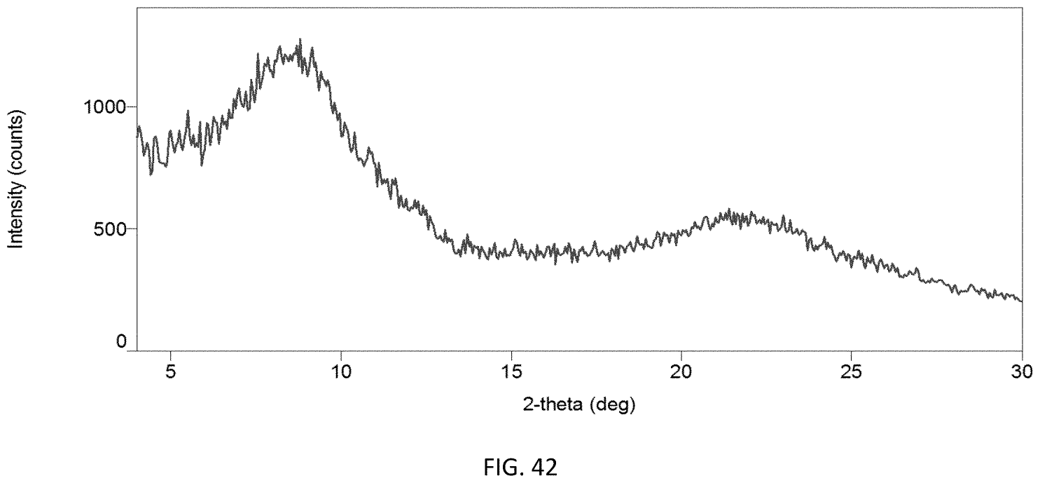

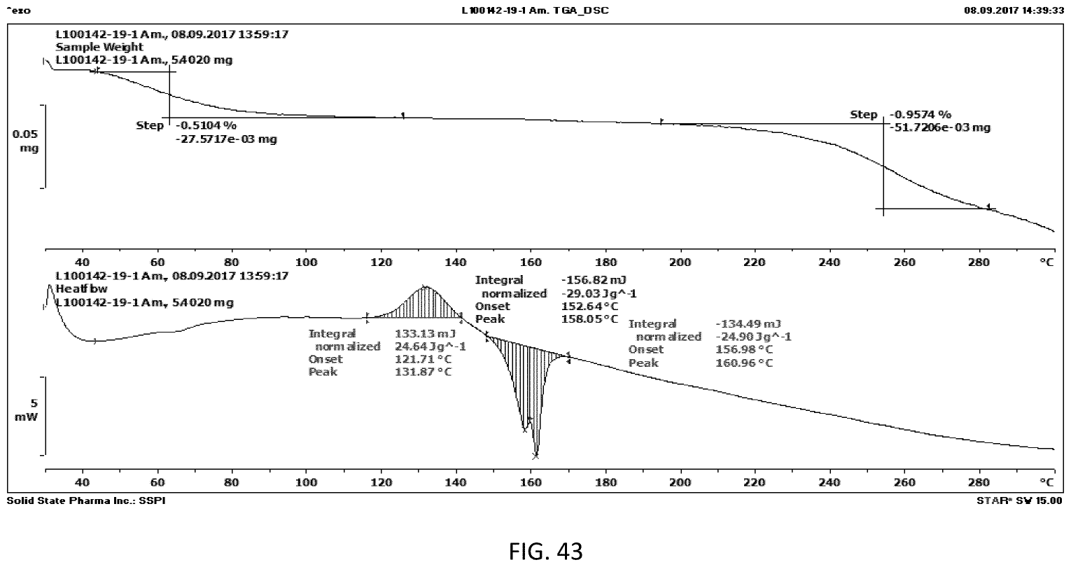

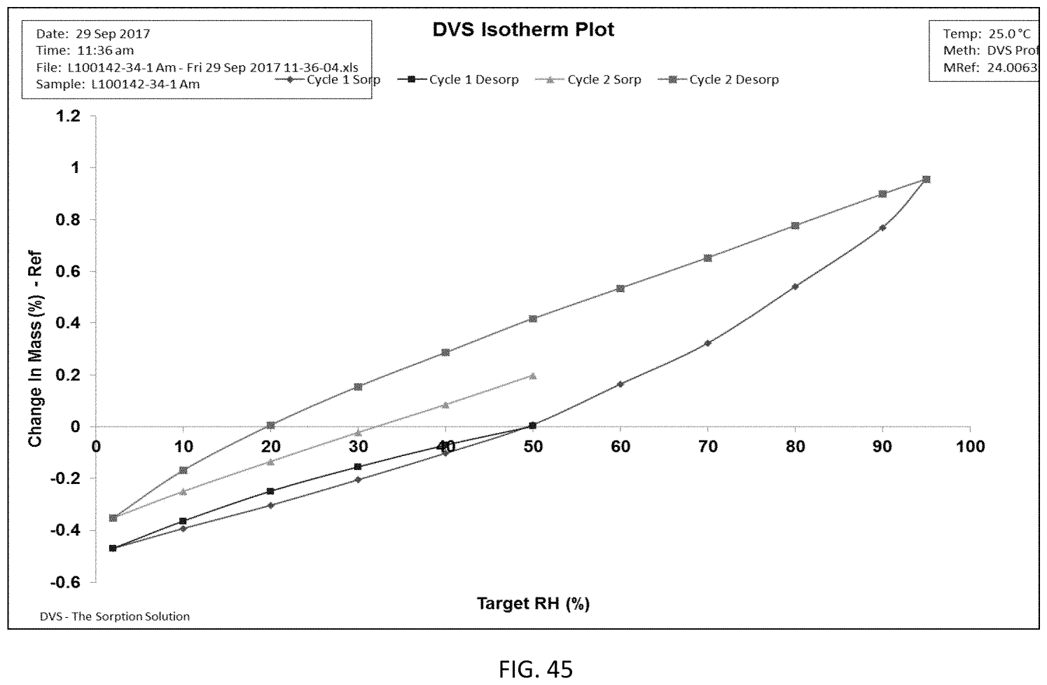

In some embodiments, a compound of formula (I) is an amorphous form. In some aspects the amorphous form exhibits an X-ray powder diffraction (XRPD) pattern substantially as shown in FIG. 42. In some aspects the amorphous form exhibits a thermogram analysis (TGA)/differential scanning calorimetry (DSC) thermogram substantially as shown in FIG. 43. The amorphous form may exhibit a dynamic vapor sorption (DVS) isotherm plot substantially as shown in FIG. 45.

In some aspects the amorphous form of a compound of formula (I) has at least one, at least two, or at least three of the following: (a) an XRPD pattern substantially as shown in FIG. 42, (b) a TGA/DSC thermogram substantially as shown in FIG. 43, and (c) a DVS isotherm plot substantially as shown in FIG. 45.

In some aspects an amorphous form of a compound of formula (I) has a solubility in water and simulated fluids in the range of 5-18 μg/mL at about 37° C. In certain aspects an amorphous form of a compound of formula (I) has a solubility in water of 5 μg/mL at about 37° C. In certain aspects an amorphous form of a compound of formula (I) has a solubility in fasted state simulated intestinal fluid (FaSSIF) of 18 μg/mL at about 37° C. In certain aspects an amorphous form of a compound of formula (I) has a solubility in fasted state simulated gastric fluid (FaSSGF) of 6 μg/mL at about 37° C.

In some embodiments, an amorphous form of compound (I) is a substantially pure amorphous form. In some aspects, “substantially pure” refers to a substance free of other substances, including polymorphic forms and/or impurities. In some aspects, the compound of formula (I) is an amorphous form and has a purity of greater than 80%, e.g., greater than 85%, greater than 90%, greater than 92.5%, greater than 95%, greater than 96%, greater than 97%, greater than 98%, greater than 99%, greater than 99.5%, greater than 99.8% (and in certain embodiments of any of the foregoing, less than 100%). In some aspects, the compound of formula (I) is an amorphous form having a purity of about 95% or at least about 95%.

Methods of Producing Polymorphs or Amorphous Forms

One or more polymorphic or amorphous forms of 2-ethylamino-8-fluoro-3-methyl-1-oxo-1,2-dihydro-isoquinoline-4-carboxylic acid ((S)-cyclopropyl-phenyl-methyl)-amide may be prepared from 2-ethylamino-8-fluoro-3-methyl-1-oxo-1,2-dihydro-isoquinoline-4-carboxylic acid ((S)-cyclopropyl-phenyl-methyl)-amide.

In some aspects, a prepared form of a compound of formula (I) is a beta form polymorph of the compound of formula (I). In some aspects a prepared form of a compound of formula (I) is a substantially pure beta form polymorph of the compound of formula (I). In some aspects, a prepared form of a compound of formula (I) is a gamma form polymorph of the compound of formula (I). In some aspects a prepared form of a compound of formula (I) is a substantially pure gamma form polymorph of the compound of formula (I). In some aspects, a prepared form of a compound of formula (I) is an alpha form polymorph of the compound of formula (I). In some aspects a prepared form a compound of formula (I) is a substantially pure alpha form polymorph of the compound of formula (I). In some aspects, a prepared form of a compound of formula (I) is an amorphous form of the compound of formula (I). In some aspects a prepared form of a compound of formula (I) is a substantially pure amorphous form of a compound of formula (I). In some aspects, a prepared form of a compound of formula (I) comprises a beta form polymorph of the compound of formula (I) and an alpha form polymorph of the compound of formula (I). In some aspects, a prepared form of a compound of formula (I) comprises a beta form polymorph of the compound of formula (I) and a gamma form polymorph of the compound of formula (I). In some aspects, a prepared form of a compound of formula (I) comprises a gamma form polymorph of the compound of formula (I) and an alpha form polymorph of the compound of formula (I). In some aspects, a prepared form of a compound of formula (I) comprises a beta form polymorph of the compound of formula (I), an alpha form polymorph of the compound of formula (I), and a gamma form polymorph of the compound of formula (I). In some aspects, a prepared form of a compound of formula (I) comprises a beta form polymorph of the compound of formula (I) and an amorphous form of the compound of formula (I).

In some embodiments methods of producing a polymorph of a compound of formula (I) comprise combining a compound of formula (I) with a solvent to produce a composition comprising one or more polymorphs of the compound of formula (I). In some aspects the solvent is a suitable solvent or a mixture of suitable solvents for producing a composition comprising one or more polymorphs of the compound of formula (I). In some aspects the solvent is selected from the group consisting of methanol, ethanol, water, isopropyl acetate, ethyl acetate, methyl tert-butyl ether, n-heptane, acetonitrile, acetone, 2-methyltetrahydrofuran, tetrahydrofuran, methyl isobutyl ketone, methyl ethyl ketone, dichloromethane, 2-propanol, 1-propanol, 1-butanol, toluene, nitromethane, methyl acetate, anisole, and any mixtures thereof.

In some embodiments methods of producing a polymorph of a compound of formula (I) include cooling crystallization, evaporative crystallization, dry and solvent drop milling, and salt disproportionation. In some aspects cooling crystallization methods include fast and slow cooling and stagnant cooling. In some aspects evaporative crystallization methods include flash evaporation, rotary evaporation, stirred evaporation, and stagnant evaporation.

In some aspects a polymorph (e.g., a beta form polymorph of the compound of formula (I)) is produced by dissolving the compound of formula (I) in one or more solvents. In some aspects the solvent is selected from the group consisting of toluene, methyl ethyl ketone, nitromethane, methyl acetate, anisole, 1-butanol, and combinations thereof. In some aspects the solvent is selected from the group consisting of 2-propanol, water, methanol, methyl isobutyl ketone, ethyl acetate, isopropyl acetate, acetone, heptane, and combinations thereof. In some aspects the solvent is selected from the group consisting of 2-propanol, ethyl acetate, acetonitrile, methyl isobutyl ketone, tert-butyl methyl ether, isopropyl acetate, ethyl acetate, 1-butanol, and combinations thereof. In some aspects the solvent is selected from the group consisting of tetrahydrofuran, acetone, methyl isobutyl ketone, and combinations thereof. In some aspects the solvent is selected from the group consisting of methyl isobutyl ketone, 2-proponal, 1-proponal, and combinations thereof.

In some aspects a polymorph (e.g., an alpha form polymorph of the compound of formula (I)) is produced by dissolving the compound of formula (I) in 1-proponal. The dissolved compound may be set at about 85-95° C., or at about 90° C. The 1-proponal may evaporate to yield a gel. The gel may then be set at about 45-55° C., or at about 50° C. until the gel crystallizes. The crystalline solid may comprise an alpha form polymorph.

In some aspects an amorphous of the compound of formula (I) is produced by melting a beta form polymorph of a compound of formula (I) to about 170-180° C., or to about 175° C. The melt is then cooled to yield a solid that is crushed to form a white, flowable powder. The obtained powder may comprise an amorphous form of the compound of formula (I).

In some aspects a polymorph (e.g., a gamma form polymorph) is produced by saturating a solution of a compound of formula (I), e.g., a solution in ethyl acetate. The saturation occurs at about room temperature. Following saturation, the composition is stored at −20° C. for one week. The composition as stored for one week may comprise a gamma form polymorph.

In some aspects a method of producing a polymorph of a compound of formula (I) comprises combining a compound of formula (I) and a solvent to produce a composition comprising one or more polymorphs of the compound of formula (I), wherein the solvent is selected from the group consisting of methanol, ethanol, water, isopropyl acetate, ethyl acetate, methyl tert-butyl ether, n-heptane, acetonitrile, acetone, 2-methyltetrahydrofuran, tetrahydrofuran, methyl isobutyl ketone, methyl ethyl ketone, dichloromethane, 2-propanol, 1-propanol, 1-butanol, toluene, nitromethane, methyl acetate, anisole, and any mixtures thereof.

In some aspects a method of producing a polymorph of a compound of formula (I) comprises combining a compound of formula (I) and a solvent to produce a slurry composition comprising one or more polymorphs of the compound of formula (I), wherein the solvent is selected from the group consisting of toluene, methyl ethyl ketone, nitromethane, methyl acetate, anisole, and 1-butanol. In some embodiments, the slurry is formed at a temperature of 22-23° C., and is maintained for 2-3 days. In some embodiments, the slurry is formed at a temperature of 50° C., and is maintained for 2 days. In some aspects, the produced polymorph of the compound of formula (I) is a beta form polymorph.

In some aspects a method of producing a polymorph of a compound of formula (I) comprises combining a compound of formula (I) and a solvent to produce a saturated solution, wherein the solvent is selected from the group consisting of 2-propanol:water (85:15), methanol:water (85:15), methyl isobutyl ketone, 2-propanol, ethyl acetate, isopropyl acetate, and acetone:heptane (2:1); and fast or slow cooling the saturated solution to form a composition comprising one or more polymorphs of the compound of formula (I). In some embodiments, the saturated solution is formed at a temperature of 50° C. In some aspects, the fast cooling of the saturated solution comprises placing the saturated solution in an ice bath and cooling the saturated solution to a temperature of about 0° C. In other aspects, the slow cooling of the saturated solution comprises cooling the saturated solution to a temperature of 22-23° C. over a period of three hours. In some aspects, the slow cooling of the saturated solution comprises reducing the temperature of the saturated solution by 5° C. per hour. In some embodiments, the produced polymorph of the compound of formula (I) is a beta form polymorph.

In some aspects a method of producing a polymorph of a compound of formula (I) comprises combining a compound of formula (I) and a solvent to produce a saturated solution, wherein the solvent is selected from the group consisting of 2-propanol, ethyl acetate, acetonitrile, methyl isobutyl ketone, tert-butyl methyl ether, isopropyl acetate, and 1-butanol; and stagnant cooling the saturated solution to form a composition comprising one or more polymorphs of the compound of formula (I). In some embodiments, the saturated solution is formed at a temperature of 22-23° C. or at a temperature of 63° C. In some embodiments, the stagnant cooling of the saturated solution comprises setting the saturated solution at −20° C. for a period of about 24 hours or for a period of about 1 to 2 weeks. In some embodiments, the stagnant cooling of the saturated solution comprises setting the saturated solution at 22-23° C. for a period of 24 hours. In some embodiments, the produced polymorph of the compound of formula (I) is selected from the group consisting of a beta form polymorph, a gamma form polymorph, and combinations thereof.

In some aspects a method of producing a polymorph of a compound of formula (I) comprises combining a compound of formula (I) and a solvent to produce a solution, wherein the solvent is selected from the group consisting of methanol, acetic acid, tetrahydrofuran, and acetone; and adding an anti-solvent to the solution dropwise to form a composition comprising one or more polymorphs of the compound of formula (I), wherein the anti-solvent is water or heptane. In some embodiments, the produced polymorph of the compound of formula (I) is a beta form polymorph.

In some aspects a method of producing a polymorph of a compound of formula (I) comprises combining a compound of formula (I) and a solvent to produce a solution, wherein the solvent is selected from the group consisting of methanol, acetic acid, tetrahydrofuran, and acetone; and adding the solution to an anti-solvent to form a composition comprising one or more polymorphs of the compound of formula (I), wherein the anti-solvent is water or heptane. In some embodiments, the produced polymorph of the compound of formula (I) is a beta form polymorph.

In some aspects a method of producing a polymorph of a compound of formula (I) comprises combining a compound of formula (I) and a solvent to produce a solution, wherein the solvent is selected from the group consisting of tetrahydrofuran, acetone, and methyl isobutyl ketone; and depositing the solution dropwise on a surface to form a gel comprising one or more polymorphs of the compound of formula (I). In some embodiments, the solution is prepared at a temperature of 50° C., and is deposited on the surface at a temperature of 120° C. The solvent may evaporate after depositing the solution on the surface. In some embodiments, the produced polymorph of the compound of formula (I) is a beta form polymorph.

In some aspects a method of producing a polymorph of a compound of formula (I) comprises combining a compound of formula (I) and a solvent to produce a solution, wherein the solvent is methyl isobutyl ketone; and evaporating the solvent using a rotary evaporator to form a composition comprising one or more polymorphs of the compound of formula (I). In some embodiments, the solvent is evaporated using a rotary evaporator at a temperature of 70° C. In some embodiments, the produced polymorph of the compound of formula (I) is a beta form polymorph.

In some aspects a method of producing a polymorph of a compound of formula (I) comprises combining a compound of formula (I) and a solvent to produce a near-saturated solution, wherein the solvent is selected from the group consisting of 2-propanol and methyl isobutyl ketone; and evaporating the solvent overnight from the near-saturated solution to form a composition comprising one or more polymorphs of the compound of formula (I), wherein the near-saturated solution is stirred during the evaporation. In some embodiments, the near-saturated solution is prepared at a temperature of 50° C. In some embodiments, the produced polymorph of the compound of formula (I) is a beta form polymorph.

In some aspects a method of producing a polymorph of a compound of formula (I) comprises combining a compound of formula (I) and a solvent to produce a solution, wherein the solvent is selected from the group consisting of 2-propanol, methyl isobutyl ketone, 1-proponal, and 1-butanol; and stagnant evaporating the solvent from the solution to form a composition comprising one or more polymorphs of the compound of formula (I). In some embodiments, the solvent is 2-propanol or methyl isobutyl ketone. In some embodiments, the solution is heated to 60° C. prior to evaporating the solvent, and the evaporation temperature is 50° C. In some embodiments, the solution is a saturated solution (e.g., at a temperature of 50° C.), and the evaporation temperature is between 40° C. and 60° C. In some embodiments, the solvent is 1-butanol or 1-proponal and the evaporation temperature is between 90° C. and 100° C. In some embodiments, the evaporation results in a gel, and the gel is set at 50° C. In some embodiments, the produced polymorph of the compound of formula (I) is selected from the group consisting of a beta form polymorph, an alpha form polymorph, and combinations thereof.

In some aspects a method of producing a polymorph of a compound of formula (I) comprises milling a compound of formula (I) three times for thirty seconds each time in a milling capsule to form a composition comprising one or more polymorphs of the compound of formula (I). In some embodiments, the compound of formula (I) is milled in combination with a solvent, and the solvent is selected from the group consisting of methanol:water (85:15), ethanol, 2-propanol, methyl isobutyl ketone, and tert-butyl methyl ether. In some embodiments, the produced polymorph of the compound of formula (I) is a beta form polymorph.

In some aspects a method of producing a polymorph of a compound of formula (I) comprises dissolving a compound of formula (I) in ethanol to produce a solution; adding an acid to the solution to form an acidic solution, wherein the acid is selected from the group consisting of benzenesulfonic acid, hydrobromic acid, hydrochloric acid, methanesulfonic acid, sulfuric acid, and toluenesulfonic acid; and adding water to the acidic solution to form a composition comprising one or more polymorphs of the compound of formula (I). In some embodiments, the method occurs at a temperature of 22-23° C., or at a temperature of 50° C. In some embodiments, the produced polymorph of the compound of formula (I) is a beta form polymorph.

In some aspects a method of producing an amorphous form of a compound of formula (I) comprises melting a compound of formula (I); and cooling the melted compound to form a solid comprising an amorphous form of the compound of formula (I). In some embodiments, the melted compound is cooled to a temperature of 25° C. In some embodiments, compound of formula (I) is heated to a temperature of 175° C. or 180° C. to melt the compound.

In some aspects a method of producing a polymorph of a compound of formula (I) comprises heating a compound of formula (I) to a temperature of 175° C. to form a melted compound; cooling the melted compound to form a solid comprising an amorphous form of the compound of formula (I); and adding a solvent to the solid to form a composition comprising one or more polymorphs of the compound of formula (I), wherein the solvent is selected from the group consisting of 2-proponal, 2-proponal:water (9:1), isopropyl acetate, tert-butyl methyl ether, water, and cyclohexane. In some embodiments, the produced polymorph of the compound of formula (I) is a beta form polymorph.

In certain aspects of any of the foregoing, the method of producing a polymorph in a method of producing a crystalline form that is substantially pure, in particular a substantially pure beta form polymorph of the compound of formula (I). In certain embodiment, the input compound is substantially free of impurities. In certain aspects, the method comprises dissolving a compound of formula (I) in acetone to form a first solution, slowly adding n-heptane to the solution to form a suspension wherein the acetone:n-heptane ratio is 1:2; heating the suspension to a temperature of about 55° C., and slowly cooling the suspension to a temperature of about -20° C. to yield beta form polymorph of the compound of formula (I).

In certain aspects, the method of producing a crystalline form that is substantially pure, in particular a substantially pure beta form polymorph of the compound of formula (I), comprises combining a compound of formula (I) and isopropyl acetate to form a first solution; distilling the first solution to replace the isopropyl acetate with acetone and to form a second solution; adding n-heptane to the second solution to form a suspension, wherein the acetone:n-heptane ratio is 1:2; heating the suspension to a temperature of about 55° C. and slowly cooling the heated suspension to a temperature of about −20° C. to yield a beta form polymorph of the compound of formula (I).

In certain embodiments, the foregoing methods of producing polymorphs include using combinations of solvents. In certain embodiments, the method of producing a substantially pure form polymorph comprises recrystallization.

Pharmaceutical Compositions

In some embodiments compositions comprise a polymorph of a compound of formula (I). In certain aspects the compositions comprise at least one, at least two, or at least three of the polymorphs described herein. In some embodiments compositions comprise an amorphous form of a compound of formula (I). In some embodiments compositions comprise an amorphous form of a compound of formula (I) and at least one polymorph of a compound of formula (I). The composition may be a pharmaceutical composition. In some embodiments, this disclosure provides compositions comprising the beta form of a compound of formula (I) as described herein.

In some embodiments a pharmaceutical composition comprises a polymorph or an amorphous form of a compound of formula (I) and a pharmaceutically acceptable carrier, diluent, or excipient. The pharmaceutical composition may comprise one or more of the forms described herein (e.g., one or more of the alpha, beta, gamma, and/or amorphous forms). In some aspects the pharmaceutical composition comprises two of the polymorphs described herein. In other aspects the pharmaceutical composition comprises three of the polymorphs described herein. In some aspects the pharmaceutical comprising comprises an amorphous form described herein. In some embodiments the compositions described herein may comprise substantially pure polymorphic or amorphous forms, or may be substantially free of other polymorphs, amorphous forms, and/or impurities. In some embodiments the pharmaceutical composition comprises a substantially pure beta form polymorph (e.g., a beta form polymorph substantially free of other polymorphs, amorphous forms, and/or impurities).

In some embodiments the term “substantially pure” or “substantially free” with respect to a particular polymorphic and amorphous form of a compound means that the composition comprising the form contains less than 95%, less than 90%, less than 80%, less than 70%, less than 60%, less than 50%, less than 40%, less than 30%, less than 20%, less than 10%, less than 5%, or less than 1% by weight of other substances, including other polymorphic forms, amorphous forms, and/or impurities. In certain embodiments, “substantially pure” or “substantially free of” refers to a substance free of other substances, including other polymorphic forms, amorphous forms, and/or impurities. Impurities may, for example, include by-products or left over reagents from chemical reactions, contaminants, degradation products, other polymorphic forms, amorphous forms, water, and solvents.

In some aspects, a polymorph of the compound of formula (I) (e.g., an alpha form, a beta form, a gamma form) has a purity of greater than 80%, e.g., greater than 85%, greater than 90%, greater than 92.5%, greater than 95%, greater than 96%, greater than 97%, greater than 98%, greater than 99%, greater than 99.5%, greater than 99.8% (and in certain embodiments of any of the foregoing, less than 100%).

In some aspects a pharmaceutical composition comprises a polymorph of a compound of formula (I) and a pharmaceutically acceptable carrier, diluent, or excipient, wherein the polymorph is an α form. In some aspects a pharmaceutical composition comprises a polymorph of a compound of formula (I) and a pharmaceutically acceptable carrier, diluent, or excipient, wherein the polymorph is a β form. In some aspects a pharmaceutical composition comprises a polymorph of a compound of formula (I) and a pharmaceutically acceptable carrier, diluent, or excipient, wherein the polymorph is a γ form. In some aspects a pharmaceutical composition comprises an amorphous form of a compound of formula (I) and a pharmaceutically acceptable carrier, diluent, or excipient. In some aspects a pharmaceutical composition comprises one or more polymorphs of a compound of formula (I) and a pharmaceutically acceptable carrier, diluent, or excipient, wherein the polymorph has a form selected from the group consisting of α form, β form, γ form, and combinations thereof.

In some aspects the pharmaceutical composition further comprises one or more additional therapeutic agents and/or active ingredients. In certain aspects a pharmaceutical composition further comprises an anti-psychotic drug. In certain aspects a pharmaceutical composition further comprises one or more drugs having as a side effect hot flashes. In certain aspects a pharmaceutical composition further comprises low-dose hormone replacement therapy. In some aspects a pharmaceutical composition further comprises one or more selective serotonin reuptake inhibitors. In some aspects a pharmaceutical composition further comprises one or more serotonin and norepinephrine reuptake inhibitors (SNRIs).

The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being comingled with an agent, and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

A pharmaceutically-acceptable carrier employed in conjunction with the compounds described herein is used at a concentration or amount sufficient to provide a practical size to dosage relationship. The pharmaceutically-acceptable carriers, in total, may, for example, comprise from about 40% to about 99.99999% by weight of the pharmaceutical compositions, e.g., from about 60% to about 99.99%, e.g., from about 80% to about 99/97%, from about 90% to about 99.95%, from about 95% to about 99.9%, or from about 98% to about 99%.

Pharmaceutically-acceptable carriers suitable for the preparation of unit dosage forms for oral administration and topical application are well-known in the art. Their selection will depend on secondary considerations like taste, cost, and/or shelf stability, which are not critical for the purposes of the subject invention, and can be made without difficulty by a person skilled in the art.

Pharmaceutically acceptable compositions can include diluents, fillers, salts, buffers, stabilizers, solubilizers and other materials which are well-known in the art. The choice of pharmaceutically-acceptable carrier to be used in conjunction with the compounds of the present invention is basically determined by the way the compound is to be administered. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof in certain embodiments. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used. Furthermore it will be appreciated that agents may be modified, e.g., with targeting moieties, moieties that increase their uptake, biological half-life (e.g., pegylation), etc.

The pharmaceutical compositions may further comprise pharmaceutically acceptable concentrations of salt, buffering agents, preservatives, compatible carriers, adjuvants, and optionally other therapeutic ingredients.

In some embodiments the pharmaceutical compositions are formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants. In some embodiments the pharmaceutical compositions are formulated as tablets or suspensions.

Uses of the Polymorph and Amorphous Forms and Compositions Thereof

In some embodiments the polymorphic and amorphous forms described herein can be administered (e.g., to a subject) alone as a pure compound. In alternative aspects the polymorphic or amorphous forms are administered in the form of a pharmaceutical composition or formulation.

In some embodiments, the present invention relates to the compositions of the present invention for use in therapy. For any of the compositions and uses described herein, in certain embodiments, the beta form of the polymorph is administered (e.g., a composition in which the beta form is the primary species of compound of formula (I)).

In some aspects disclosed herein are methods of treating or preventing a disease or disorder in a subject by administering a polymorph or amorphous form of the compound of formula (I). In some aspects, a pharmaceutical composition comprises one or more polymorphs of the compound of formula (I). In some aspects, a pharmaceutical composition comprises an amorphous form of the compound of formula (I). In some aspects, a therapeutically effective amount of the pharmaceutical composition is administered to the subject. The disorder or disease may be selected from psychosis; schizophrenia; schizophrenoform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; major depression; general anxiety disorder; bipolar disorder (maintenance treatment, recurrence prevention and stabilization); mania; hypomania; cognitive impairment; ADHD; obesity; appetite reduction; excess body weight; excess body fat; cognitive disorders; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; PTSD; dementia and agitation and delirium in the elderly; inflammatory diseases including irritable bowel syndrome and inflammatory bowel disorders; emesis; pre-eclampsia; airway hyperresponsiveness; reproduction disorders and sex hormone-dependent diseases including but not limited to benign prostatic hyperplasia (BPH), metastatic prostatic carninoma, testicular cancer, breast cancer, androgen dependent acne, male pattern baldness, endometriosis, abnormal puberty, uterine fibrosis, hormone-dependent cancers, hyperandrogenism, hirsutism, virilization, polycystic ovary syndrome (PCOS), HAIR-AN syndrome (hyperandrogenism, insulin resistance and acanthosis nigricans), ovarian hyperthecosis (HAIR-AN with hyperplasia of luteinized theca cells in ovarian stroma), other manifestations of high intraovarian androgen concentrations (e.g. follicular maturation arrest, atresia, anovulation, dysmenorrhea, dysfunctional uterine bleeding, infertility) and androgen-producing tumor (virilizing ovarian or adrenal tumor); gynecological disorders and infertility.

In some aspects disclosed herein are methods of treating or preventing hot flashes in a subject by administering a polymorph or amorphous form of the compound of formula (I). In some aspects the hot flashes occur as a result of menopause, removal of ovaries or tests, treatment for breast cancer, androgen deprivation therapy, hypogonadism and low serum gonadotropin levels, leukemia, non-dipper hypertension, carcinoid syndrome, post-menopausal hyperandrogenism, or precocious puberty in males and females. In some aspects the hot flashes are drug-induced hot flashes. In some aspects, the method of treating or preventing is in a subject in need thereof. In some aspects, the subject is a post-menopausal or peri-menopausal female. In some aspects, the method of treating or preventing hot flashes is by administering any of the compounds or pharmaceutical compounds of the disclosure, such as beta form polymorph, in a form suitable for once daily dosing (or in accordance with a dosing regimen for once daily dosing).

In some aspects disclosed herein are methods of treating gynecological disorders and infertility in a subject by administering a polymorph or amorphous form of the compound of formula (I). In some aspects the methods include suppressing the LH-surge in assisted conception. In some embodiments the compounds are administered to cause male castration and/or to inhibit the sex drive in men.

In some aspects disclosed herein are methods for treating an excess of body fat and/or excess body weight (e.g., treating, preventing, arresting, and/or reducing weight gain) in a subject by administering a polymorph or amorphous form of the compound of formula (I). In some aspects the methods of treating an excess of body fat and/or excess body weight in a subject includes decreasing body fat and/or body weight; preventing weight gain and/or ceasing weight gain; decreasing or maintaining plasma triglyceride levels; improving leptin resistance; reducing hyperglycemia and/or decreasing incidence or severity of diabetes; reducing hyperlipidaemia and/or hypertriglyceridemia; decreasing food intake; improving at least one condition associated with weight gain including a cardiovascular disorder, a sleep disorder, a metabolic condition, or a diabetes-related condition; at least partially improving (e.g., terminating or reducing in occurrence) a condition selected from binge eating disorder, night eating syndrome, obsessive eating, compulsive eating, or bulimia; preventing or decreasing abdominal fat accumulation.

In some aspects disclosed herein are methods for treating or preventing a leptin-related disease in a subject by administering a polymorph or amorphous form of the compound of formula (I). Examples of leptin-related diseases include metabolic disorders such as diabetes (e.g., Type 1 diabetes), cardiovascular diseases or metabolic syndrome; lipid regulation disorders such as lipodystrophy, including congenital and acquired lipodystrophy, dyslipidemia, nonalcoholic fatty liver disease (NAFLD), nonalcoholic steatohepatitis (NASH) or hyperlipidemia; Congenital Leptin Deficiency (CLD); hypothalamic amenorrhea, such as exercise-induced hypothalamic amenorrhea; Rabson-Mendenhall syndrome; and osteoporosis.

In some aspects disclosed herein are methods for treating a subject suffering from a hormonal imbalance by administering a polymorph or amorphous form of the compound of formula (I). Subjects who may be suffering from a hormonal imbalance include women subjected to estrogen-lowering therapies, for example for treatment of breast, cervical, or uterine cancers, or for the treatment of women's health disorders such as endometriosis, uterine fibroids, heavy menstrual bleeding and polycystic ovary syndrome (PCOS); women experiencing natural, age-related decreases in estrogens as occurring during peri-menopause and post-menopause; men subjected to androgen-lowering therapies, such as for treatment of prostate-cancer or benign prostatic hyperplasia (BPH); and men experiencing natural, age-related decreases in circulating testosterone.

In some embodiments the disclosure provides a method for the treatment of schizophrenia, the method comprising the administration of a therapeutically effective amount of a polymorph or amorphous form of the compound of formula (I) to a patient in need thereof. In particular, said treatment includes the treatment of the positive, negative and/or cognitive symptoms of schizophrenia.

In some embodiments the disclosure provides a method of treating cognitive impairment, the method comprising the administration of a therapeutically effective amount of a polymorph or amorphous form of the compound of formula (I) to a patient in need thereof. In particular, said cognitive impairment is manifested as a decline in working memory, attention and vigilance, verbal learning and memory, visual learning and memory, reasoning and problem solving e.g. executive function, speed of processing and/or social cognition.

In some embodiments a polymorph or amorphous form of the compound of formula (I) is administered as part of a combination therapy. In some aspects the combination therapy includes co-administration of the polymorph or amorphous form with a therapeutic agent. In other aspects the combination therapy includes sequential administration of the polymorph or amorphous form and the therapeutic agent.

In some aspects a polymorph or amorphous form of the compound of formula (I) is administered in combination with D2 antagonists. In some aspects a polymorph or amorphous form of the compound of formula (I) is administered in combination with antagonists/inverse agonists/negative modulators/partial agonists of one or more of the targets dopamine D2 receptor, dopamine D3 receptor, dopamine D4 receptor, phosphodiesterase PDE10, serotonin 5-HT_{1A} receptor, serotonin 5-HT_{2A} receptor, serotonin 5-HT₆ receptor, adrenergic alpha 2 receptor, cannabinoid type 1 receptor, histamine H3 receptor, cyclooxygenases, sodium channels or glycine transporter GlyT1; or with agonists/positive modulators/partial agonists of one or more of the targets serotonin 5-HT_{2C} receptor, KCNQ channels, NMDA receptor, AMPA receptor, nicotinic alpha-7 receptor, muscarinic M1 receptor, muscarinic M4 receptor, metabotropic glutamate receptor mGluR2, metabotropic glutamate receptor mGluR5, dopamine D1 receptor or dopamine D5 receptor.

Such combined administration of a polymorph or amorphous form of the compound of formula (I) and other anti-psychotic compounds may be sequential or concomitant. Examples of D2 antagonists or partial agonists include haloperidol, chlorpromazine, sulpirid, risperidone, ziprasidon, olanzapine, quetiapin, clozapine and aripiprazole.

In some aspects a polymorph or amorphous form of the compound of formula (I) is administered in combination with low-dose hormone replacement therapy. In certain aspects the polymorph or amorphous form increases the efficacy of the low-dose hormone replacement therapy.

In some aspects a polymorph or amorphous form of the compound of formula (I) is administered in combination with one or more selective serotonin reuptake inhibitors. In some aspects a polymorph or amorphous form of the compound of formula (I) is administered in combination with one or more serotonin and norepinephrine reuptake inhibitors (SNRIs).

In some aspects a polymorph or amorphous form of the compound of formula (I) is administered in combination with a therapeutic agent or drug that has as a side effect hot flashes.

In some aspects, a polymorph or amorphous form of the compound of formula (I) is administered in an amount from about 0.001 mg/kg body weight to about 100 mg/kg body weight per day. In particular, daily dosages may be in the range of 0.01 mg/kg body weight to about 50 mg/kg body weight per day. The exact dosages will depend upon the frequency and mode of administration, the sex, the age the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 1-1000 mg/day of a compound of the present invention, such as 1-500 mg/day, such as 1-100 mg/day, such as 1-50 mg/day, such as 2-20 mg/day, or such as or 10-30 mg/day (e.g., 10 mg/day, 15 mg/day, 20 mg/day, 22.5 mg/day, 25 mg/day, or 30 mg/day).

In one embodiment the present invention relates to the use of a polymorph or amorphous form of the compound of formula (I) in the manufacture of a medicament for the treatment of a disease. In some aspects, the disease is selected from psychosis; schizophrenia; schizophrenoform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; major depression; general anxiety disorder; bipolar disorder (maintenance treatment, recurrence prevention and stabilization); mania; hypomania; cognitive impairment; ADHD; obesity; appetite reduction; cognitive disorders; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; PTSD; dementia and agitation and delirium in the elderly; inflammatory diseases including irritable bowel syndrome and inflammatory bowel disorders; emesis; pre-eclampsia; airway hyperresponsiveness; reproduction disorders and sex hormone-dependent diseases including but not limited to benign prostatic hyperplasia (BPH), metastatic prostatic carninoma, testicular cancer, breast cancer, androgen dependent acne, male pattern baldness, endometriosis, abnormal puberty, uterine fibrosis, hormone-dependent cancers, hyperandrogenism, hirsutism, virilization, polycystic ovary syndrome (PCOS), HAIR-AN syndrome (hyperandrogenism, insulin resistance and acanthosis nigricans), ovarian hyperthecosis (HAIR-AN with hyperplasia of luteinized theca cells in ovarian stroma), other manifestations of high intraovarian androgen concentrations (e.g. follicular maturation arrest, atresia, anovulation, dysmenorrhea, dysfunctional uterine bleeding, infertility) and androgen-producing tumor (virilizing ovarian or adrenal tumor); gynecological disorders and infertility.

In some embodiments a polymorph or amorphous form of the compound of formula (I) is used in the manufacture of a medicament for treating or preventing hot flashes. In some aspects the hot flashes occur as a result of menopause, removal of ovaries or tests, treatment for breast cancer, androgen deprivation therapy, hypogonadism and low serum gonadotropin levels, leukemia, non-dipper hypertension, carcinoid syndrome, post-menopausal hyperandrogenism, or precocious puberty in males and females. In some aspects the hot flashes are drug-induced hot flashes.

In some embodiments a polymorph or amorphous form of the compound of formula (I) is used in the manufacture of a medicament for treating or preventing gynecological disorders and infertility in a subject by administering a polymorph or amorphous form of the compound of formula (I).

In some embodiments a polymorph or amorphous form of the compound of formula (I) is used in the manufacture of a medicament for treating or preventing an excess of body fat and/or excess body weight (e.g., treating, preventing, arresting, and/or reducing weight gain).

In some embodiments a polymorph or amorphous form of the compound of formula (I) is used in the manufacture of a medicament for treating or preventing a leptin-related disease. Examples of leptin-related diseases include metabolic disorders such as diabetes (e.g., Type 1 diabetes), cardiovascular diseases or metabolic syndrome; lipid regulation disorders such as lipodystrophy, including congenital and acquired lipodystrophy, dyslipidemia, nonalcoholic fatty liver disease (NAFLD), nonalcoholic steatohepatitis (NASH) or hyperlipidemia; Congenital Leptin Deficiency (CLD); hypothalamic amenorrhea, such as exercise-induced hypothalamic amenorrhea; Rabson-Mendenhall syndrome; and osteoporosis.

In some embodiments a polymorph or amorphous form of the compound of formula (I) is used in the manufacture of a medicament for treating a subject suffering from a hormonal imbalance. Subjects who may be suffering from a hormonal imbalance include women subjected to estrogen-lowering therapies, for example for treatment of breast, cervical, or uterine cancers, or for the treatment of women's health disorders such as endometriosis, uterine fibroids, heavy menstrual bleeding and polycystic ovary syndrome (PCOS); women experiencing natural, age-related decreases in estrogens as occurring during peri-menopause and post-menopause; men subjected to androgen-lowering therapies, such as for treatment of prostate-cancer or benign prostatic hyperplasia (BPH); and men experiencing natural, age-related decreases in circulating testosterone.

In some embodiments a polymorph or amorphous form of the compound of formula (I) is used in the manufacture of a cosmetic treatment to stimulate the loss of body weight and/or of body fat in a subject. The “cosmetic treatment” is intended to provide an aesthetic/cosmetic effect in subjects, by improving body appearance through stimulating the loss of body weight and/or of body fat (e.g., reduce cellulite). It enables subjects to stabilize weight and to stay thin without localized fat deposits.

In one embodiment the present invention relates to the use of a compound of the present invention in the manufacture of a medicament for the treatment of schizophrenia. In particular, said treatment includes the treatment of the positive, negative and/or cognitive symptoms of schizophrenia.

In some embodiments the present invention relates to the use of a compound as described herein in the manufacture of a medicament to suppress the LH-surge in assisted conception in a patient.

In some embodiments the present invention relates to the use of a compound as described herein in the manufacture of a medicament to treat and/or prevent leptin-related diseases.

In one embodiment, the present invention relates to the use of a compound of the present invention in the manufacture of a medicament for the treatment of cognitive impairment. In particular, said cognitive impairment is manifested as a decline in working memory, attention and vigilance, verbal learning and memory, visual learning and memory, reasoning and problem solving e.g. executive function, speed of processing and/or social cognition.

In one embodiment, the present invention relates to a compound of the present invention for use in the treatment of a disease selected from psychosis; schizophrenia; schizophrenoform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; major depression; general anxiety disorder; bipolar disorder (maintenance treatment, recurrence prevention and stabilization); mania; hypomania; cognitive impairment; ADHD; obesity; appetite reduction; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; gut inflammation; inflammatory bowel syndrome; PTSD; dementia and agitation and delirium in the elderly.

In one embodiment, the present invention relates to a compound of the present invention for use in the treatment of schizophrenia. In particular, said treatment includes the treatment of the positive, negative and/or cognitive symptoms of schizophrenia.

In one embodiment, the present invention relates to a compound of the present invention for use in the treatment of cognitive impairment. In particular, said cognitive impairment is manifested as a decline in working memory, attention and vigilance, verbal learning and memory, visual learning and memory, reasoning and problem solving e.g. executive function, speed of processing and/or social cognition.

In one embodiment, the invention relates to a pharmaceutical composition comprising a composition of the present invention together with an anti-psychotic agent. In one embodiment, said anti-psychotic agent is selected from antagonists/inverse agonists/negative modulators/partial agonists of the targets dopamine D2 receptor, dopamine D3 receptor, dopamine D4 receptor, phosphodiesterase PDE10, serotonin 5-HT_{1A} receptor, serotonin 5-HT_{2A} receptor, serotonin 5-HT₆ receptor, adrenergic alpha 2 receptor, cannabinoid type 1 receptor, histamine H3 receptor, cyclooxygenases, sodium channels or glycine transporter GlyT1; or from agonists/positive modulators/partial agonists of the targets serotonin 5-HT_{2C} receptor, KCNQ channels, NMDA receptor, AMPA receptor, nicotinic alpha-7 receptor, muscarinic M1 receptor, muscarinic M4 receptor, metabotropic glutamate receptor mGluR2, metabotropic glutamate receptor mGluR5, dopamine D1 receptor or dopamine D5 receptor. Particular examples of such anti-psychotics include haloperidol, chlorpromazine, sulpirid, risperidone, ziprasidon, olanzapine, quetiapine, clozapine and aripoprazole.

The polymorphs described herein may be administered in pharmaceutically acceptable solutions, which may routinely contain pharmaceutically acceptable concentrations of salt, buffering agents, preservatives, compatible carriers, adjuvants, and optionally other therapeutic ingredients.

The polymorph or amorphous forms of the compound of formula (I) may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, polymorph or amorphous forms of the compound of formula (I) may be administered directly to a tissue. Direct tissue administration may be achieved by direct injection. The polymorph or amorphous forms may be administered once, or alternatively they may be administered in a plurality of administrations. If administered multiple times, the polymorph or amorphous forms may be administered via different routes. For example, the first (or the first few) administrations may be made directly into the affected tissue while later administrations may be systemic.

For oral administration, compositions can be formulated readily by combining the polymorph or amorphous forms with pharmaceutically acceptable carriers well known in the art. Such carriers enable the polymorph or amorphous forms to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, emulsions, and other non-aqueous vehicles. Examples of non-aqueous solvents are propylene glycol, polyethylene glycol, vegetable oils such as olive oil, and injectable organic esters such as ethyl oleate. Additional examples of non-aqueous vehicles for delivery include 10% 2-hydroxypropyl-β-cyclodextrin (HPbetaCD), 25% polyethylene glycol (15)-hydroxystearate (Solutol HS15®), 25% polyoxyl 35 hydrogenated castor oil (Cremophor EL®), 100% macrogol 6 glycerol caprylocaprate (Softigen 767®); 100% polyethylene glycol (PEG 400), 100% Viscoleo, and 90:10 Gelucire® 44/14:PEG 400.

Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include suppositories, sprays, ointments, cremes, gels, inhalants, dermal patches, implants, etc. Transdermal patches may be placed on the skin and used to deliver a specific dose of medication through the skin and into the bloodstream. In some aspects a transdermal patch provides for controlled release of the pharmaceutical composition.

Conveniently, the compounds of the invention are administered in a unit dosage form containing said compounds in an amount of about 0.1 to 500 mg, such as 1 mg, 5 mg, 7.5 mg, 10 mg, 20 mg, 50 mg 100 mg, 150 mg, 200 mg or 250 mg of a compound of the present invention.

In certain embodiments a polymorph described herein (e.g., a beta form polymorph) is formulated in a solid dosage form, such as a tablet. The polymorph may be micronized prior to formulating as a tablet. In some aspects the tablet comprises the polymorph in an amount of 7-10 mg, or the tablet may comprise the polymorph in an amount of 7.5 mg or 10 mg. In some aspects a polymorph (e.g., a beta form polymorph) is administered once a day to a subject in one or more tablets comprising a total amount of polymorph of 7.5 mg, 10 mg, 15 mg, 17.5 mg, 20 mg, 22.5 mg, or 25 mg. In certain aspects the polymorph is a substantially pure beta form polymorph (e.g., substantially free of impurities, other polymorphs, or amorphous forms). The substantially pure beta form polymorph may have a purity of greater than 95% or greater than 99%. In certain aspects, the disclosure provide a solid dosage form, such as a tablet, in which the active pharmaceutical ingredient is the beta form polymorph (as described herein) of a compound of Formula (I), which may be micronized and may be substantially pure.

For parenteral routes such as intravenous, intrathecal, intramuscular and similar administration, typically doses are in the order of about half the dose employed for oral administration.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospho lipids, fatty acids, fatty acid amines, polyoxyethylene and water. The pharmaceutical compositions formed by combining the compound of the invention and the pharmaceutical acceptable carriers are then readily administered in a variety of dosage forms suitable for the disclosed routes of administration.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablet, e.g. placed in a hard gelatine capsule in powder or pellet form or in the form of a troche or lozenge. The amount of solid carrier may vary but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants and/or diluents followed by the compression of the mixture in a conventional tabletting machine. Examples of adjuvants or diluents comprise: Corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

Kits

The polymorph and/or amorphous forms described herein can be provided in a kit. The kit includes (a) one or more polymorphs and/or amorphous forms of the compound of formula (I), e.g., a composition that includes the one or more compounds and (b) informational material. In some embodiments the kit further includes an anti-psychotic drug. In some aspects the anti-psychotic drug is selected from typical anti-psychotics, atypical anti-psychotics, antagonists/inverse agonists/negative modulators/partial agonists of one or more of the targets dopamine D2 receptor, dopamine D3 receptor, dopamine D4 receptor, phosphodiesterase PDE10, serotonin 5-HT_{1A} receptor, serotonin 5-HT_{2A} receptor, serotonin 5-HT₆ receptor, adrenergic alpha 2 receptor, cannabinoid type 1 receptor, histamine H3 receptor, cyclooxygenases, sodium channels or glycine transporter GlyT1; or with agonists/positive modulators/partial agonists of one or more of the targets serotonin 5-HT_{2C} receptor, KCNQ channels, NMDA receptor, AMPA receptor, nicotinic alpha-7 receptor, muscarinic M1 receptor, muscarinic M4 receptor, metabotropic glutamate receptor mGluR2, metabotropic glutamate receptor mGluR5, dopamine D1 receptor or dopamine D5 receptor.

Particular examples of such anti-psychotics include haloperidol, chlorpromazine, sulpirid, risperidone, ziprasidon, olanzapine, quetiapine, clozapine and aripiprazole.

The informational material can be descriptive, instructional, marketing or other material that relates to the methods described herein and/or the use of the components for the methods described herein. For example, the informational material may describe methods for administering the compounds, and in some aspects the anti-psychotic drugs, to a subject. The informational material can include instructions to administer the compounds described herein in a suitable manner, e.g., in a suitable dose, dosage form, or mode of administration. In some embodiments, the instructions recommend administering an effective amount of a compound. The informational material can include instructions for selecting a suitable subject.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the inhibitor and/or its use in the methods described herein. Of course, the informational material can also be provided in any combination of formats.

In addition to the compounds and, in some aspects the anti-psychotic drug, the kit can include other ingredients, such as a solvent or buffer, a stabilizer or a preservative, and/or an agent for treating a condition or disorder described herein. Alternatively, the other ingredients can be included in the kit, but in different compositions or containers than the compound. In such embodiments, the kit can include instructions for admixing the compound and the other ingredients, or for using the compound with the other ingredients.

The compound described herein can be provided in any form, e.g., liquid, dried or lyophilized form. It is preferred that the glutamine metabolism inhibitor be substantially pure and/or sterile. When the compound is provided in a liquid solution, the liquid solution preferably is an aqueous solution, with a sterile aqueous solution being preferred. When the compound is provided as a dried form, reconstitution generally is by the addition of a suitable solvent. The solvent, e.g., sterile water or buffer, can optionally be provided in the kit.

The kit can include one or more containers for the composition containing the compound and an anti-psychotic drug. In some embodiments, the kit contains separate containers, dividers or compartments for the compound (e.g., in a composition), the anti-psychotic drug, and informational material. For example, the compound and the anti-psychotic drug can each be contained in a bottle, vial, or syringe, and the informational material can be contained in a plastic sleeve or packet. In other embodiments, the separate elements of the kit are contained within a single, undivided container. For example, the compound (e.g., in a composition) and the anti-psychotic drug are contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the compound (e.g., in a composition) and the anti-psychotic drug. For example, the kit includes a plurality of syringes, ampules, foil packets, or blister packs, each containing a single unit dose of the agent. The containers of the kits can be air tight and/or waterproof.

All patents and other publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following example illustrates some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### pKa and Log P/D Experimental Determination of SJB-01

pKa Determination of SJB-01

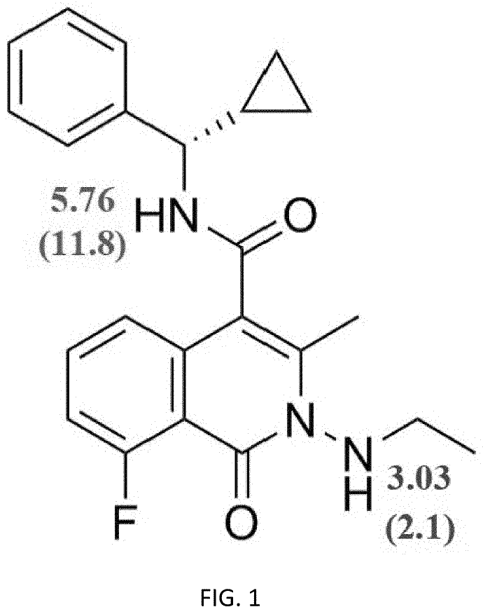

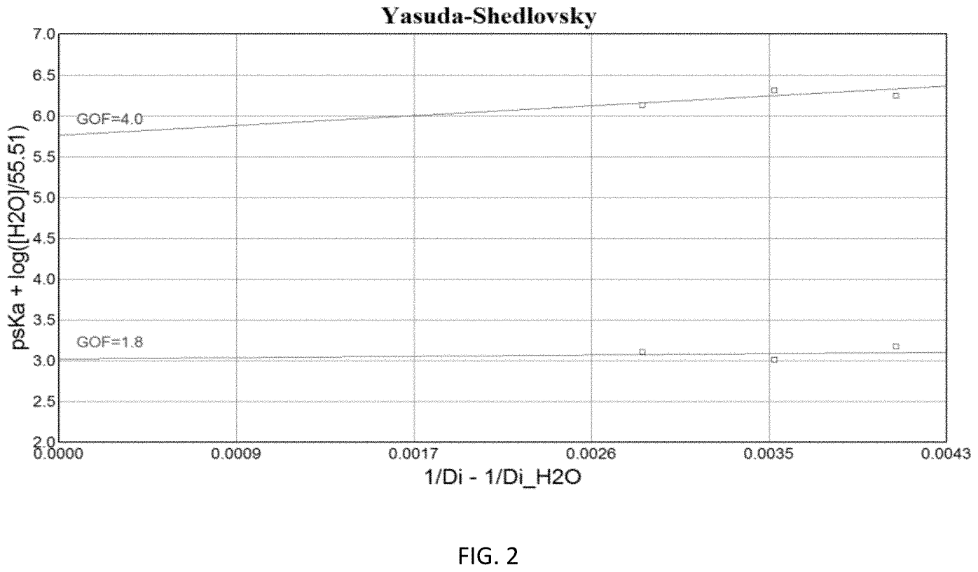

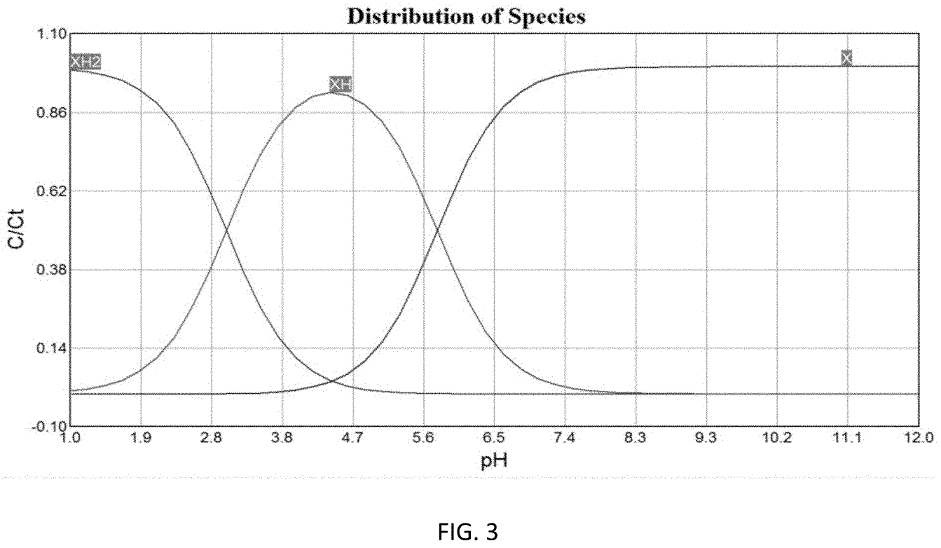

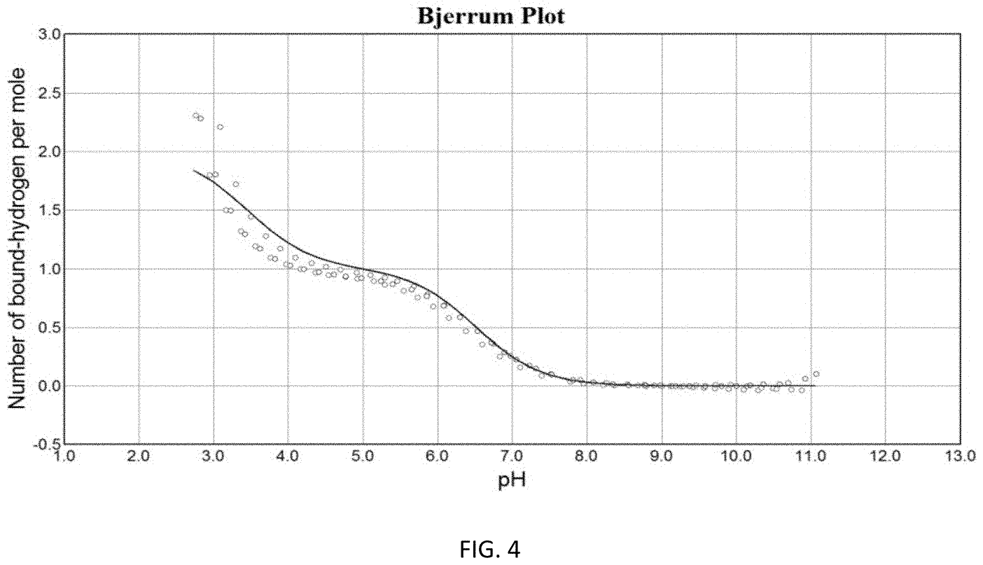

pKa determination and assignment was completed on SJB-01 (FIG. 1). About 2 mg of SJB-01 (100142-1-1) was titrated three times with varying concentrations of MeOH as co-solvent to generate a Yasuda-Shedlovsky plot (L100142-65-4, FIG. 2 and Table 1). Extrapolation of the Yasuda-Shedlovsky plot to 0% co-solvent determined the pKa's (FIG. 3), which are acidic 5.76, basic 3.03. Acidic pKa is in red and basic pKa is in blue. The Bjerrum plot shows good correlation of experimental data fitted to the theoretical model (FIG. 4).

TABLE 1
pKa Data of SJB-01 (L100142-69-1).

| | | Temperature | | |
|---|---|---|---|---|
| Type | pKa | ° C. | Wt. % MeOH | Method |
| Acidic | 5.76 ± 0.11 | 25.4 | 41.66 | co-solvent |
| Basic | 3.03 ± 0.11 | | 37.10 | potentiometric |
| | | | 31.68 | |

Log P/D Determination of SJB-01

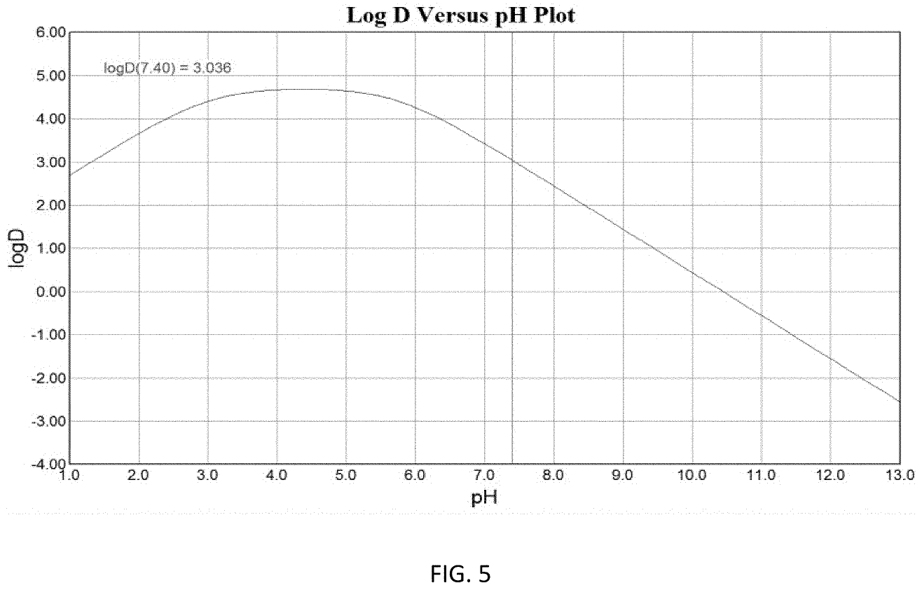

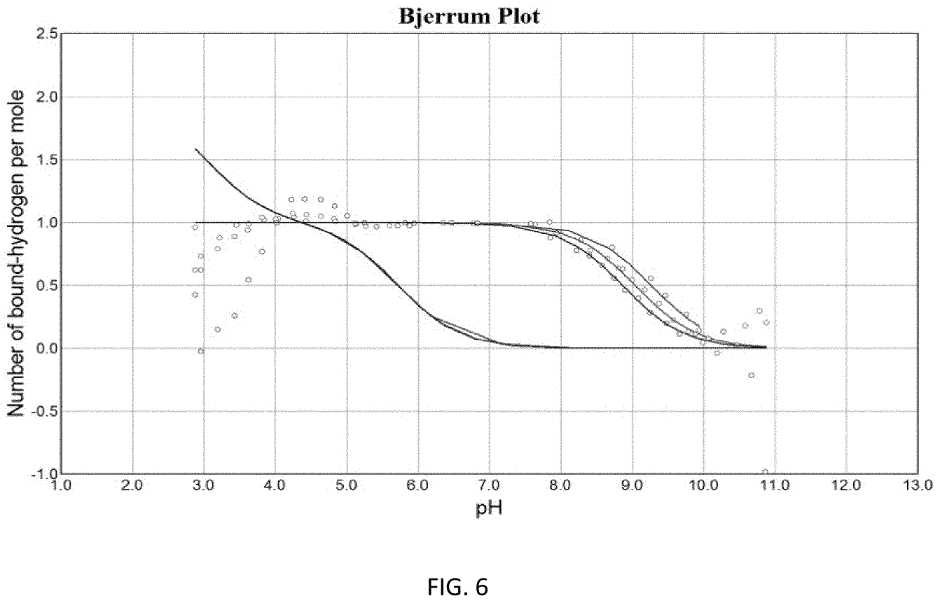

Log P and log D were determined for SJB-01 free base using 1-octanol as the partition solvent (Table 2). Log P was experimentally determined to be 4.73 which is larger than the predicted value for the structure, the corresponding Bjerrum plot is shown in FIG. 6. A log D profile (FIG. 5) was constructed for SJB-01 based on the distribution of the API at a particular pH.

There are challenges with performing log P experiments of SJB-01 due to its low water solubility across the pH range.

TABLE 2
Log-P of SJB-01 neutral species - grouped after three titrations.

| Partition | | Temperature | Octanol:water | |
|---|---|---|---|---|
| Equilibria | LogP | (° C.) | ratio (mL) | Method |
| XH═XH* | 4.73 ± 0.14 | 25.3 | 0.4:6 | pH-metric |
| | | | 0.4:10 | |
| | | | 0.4:15 | |

pKa and Log P Theoretical Calculations

Based on the theoretical calculations, the following assignments and values were determined:

Strongest pKa (base): 2.1±0.9

Strongest pKa (acid): 11.8±1.0

pKa Value Contribution of Microstages

Statically the microstages (MS) contain microstates that play a role in the overall thermodynamic fluctuations of a molecule which contribute to the observed macrostate of SJB-01. The following gives the predicted pKa values for SJB-01 with the corresponding microstage and influence associated with each value:

2.1±0.9 100% MS1

11.8±1.0 100% MS2

MS1 and MS2 consist of the following contributions:

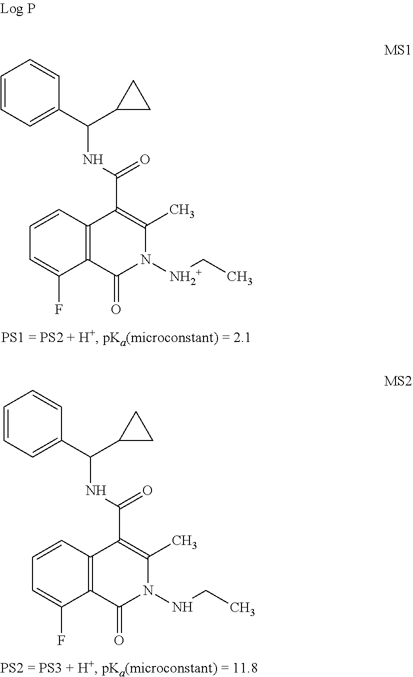 [SMILES: CC[NH2+]n1c(C)c(C(=O)NC(c2ccccc2)C2CC2)c2cccc(F)c2c1=O ; CCNn1c(C)c(C(=O)NC(c2ccccc2)C2CC2)c2cccc(F)c2c1=O]

Log P for SJB-01 was predicted to the following values based on the characteristic atomic or fragment regions of the molecule. Two calculations were conducted, and the predictions are as follows:

3.26 (moderate reliability, RI=0.69)

4.06±0.75

Instrumentation

pKa, Log P/D Titrations

pKa and log P/D data were collected using Pion PULSE Attain software. The Pion PULSE solutions consisted of using ionic strength adjusted water (0.15 M KCl), 0.5 M HCl and 0.5 M NaOH which were used for all titrations. Purge gas (argon) was used for HEPES (probe validation), KHP (KOH concentration determination), aqueous potentiometric and log P/D potentiometric titrations.

The Pion PULSE was equipped with an overhead variable speed stirrer, double junction Ag/AgCl pH probe, UV probe with a 1 cm pathlength, Carl-Zeiss spectrophotometer with 256-bit photodiode array, and a temperature probe. The pH of the instrument can range from 1.8-12.2. The compatible co-solvents are MeOH, EtOH, ACN, DMSO and 1,4-dioxane. pKa measurements were completed by either potentiometric or UV-metric methods. log P/D was measured using water saturated 1-octanol as the partitioning solvent. Data was refined using the Pion PULSE refine software.

Parameters
Experiment 1: pKa Determination

| Method | Co-solvent potentiometric |
|---|---|
| Solvent used | 80% vol. ionic strength adjusted MeOH |
| Sample amount | 1-3 mg |
| Titration direction | Alternating starting low-high (up-down-up) |
| pH range | 2-11 |

Experiment 2: Log P/D Determination

| | |
|---|---|
| Method | Aqueous potentiometric |
| Partitioning solvent | 1-octanol |
| Sample amount | 1-2 mg |
| Titration direction | Low-high (up only) |
| pH range | 3-11 |

### Example 2

### Salt Screening and Polymorph Screening of SJB-01

Baseline Characterization

Baseline characterization of SJB-01 was carried out on lot number 2223168, (L100142-1-1) and included x-ray powder diffraction (XRPD), simultaneous thermogravimetric analysis and differential scanning calorimetry (TGA/DSC), dynamic vapor sorption (DVS) and optical microscopy. A second batch of slightly lower purity SJB-01, lot number 17-12607, (L100142-21-1) was utilized to carry out additional screening experiments to determine the effect of impurities on the propensity for polymorphism. The purity of the two batches were compared by high performance liquid chromatography (HPLC).

X-ray Powder Diffraction (XRPD)

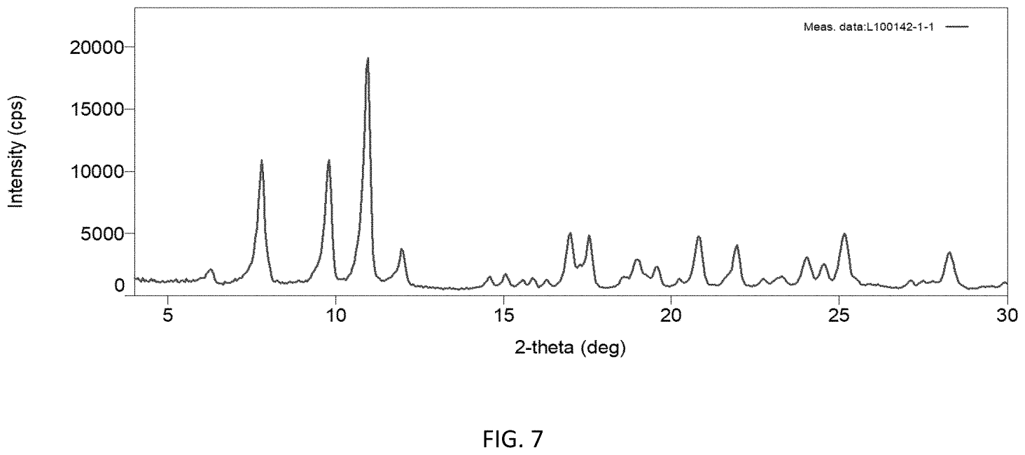

XRPD was done on SJB-01 freebase (lot#2223168, L100142-1-1). The SJB-01 freebase was a solid sample that was shown to be crystalline by the XRPD. The pattern exhibited by the material (FIG. 7) was found to be Pattern β.

Thermogravimetric Analysis and Differential Scanning Calorimetry (TGA/DSC)

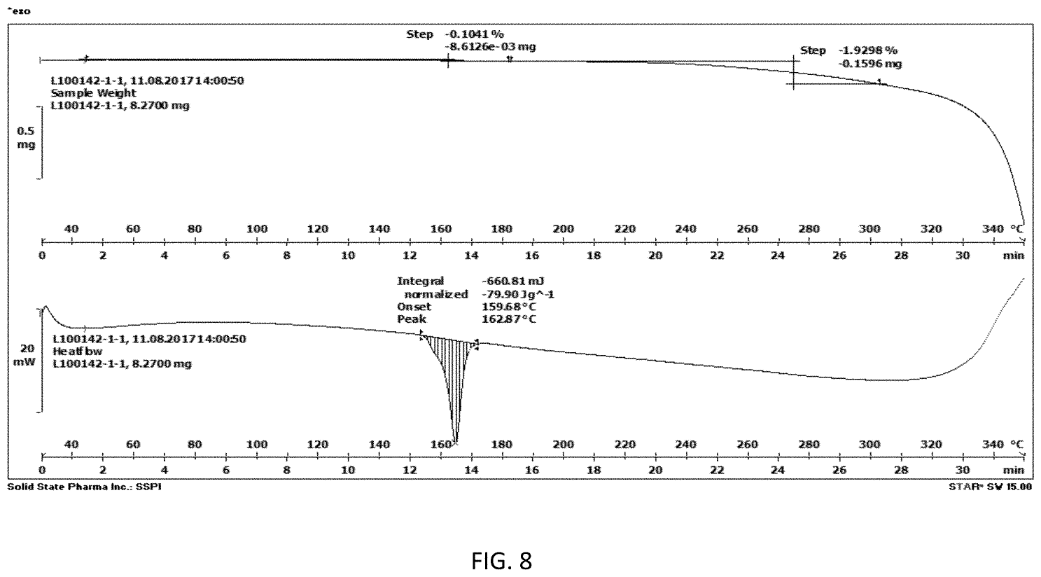

The solid had a melting onset temperature of about 159° C. with a peak of about 162.9° C. The solid was anhydrous (FIG. 8).

Dynamic Vapor Sorption (DVS)

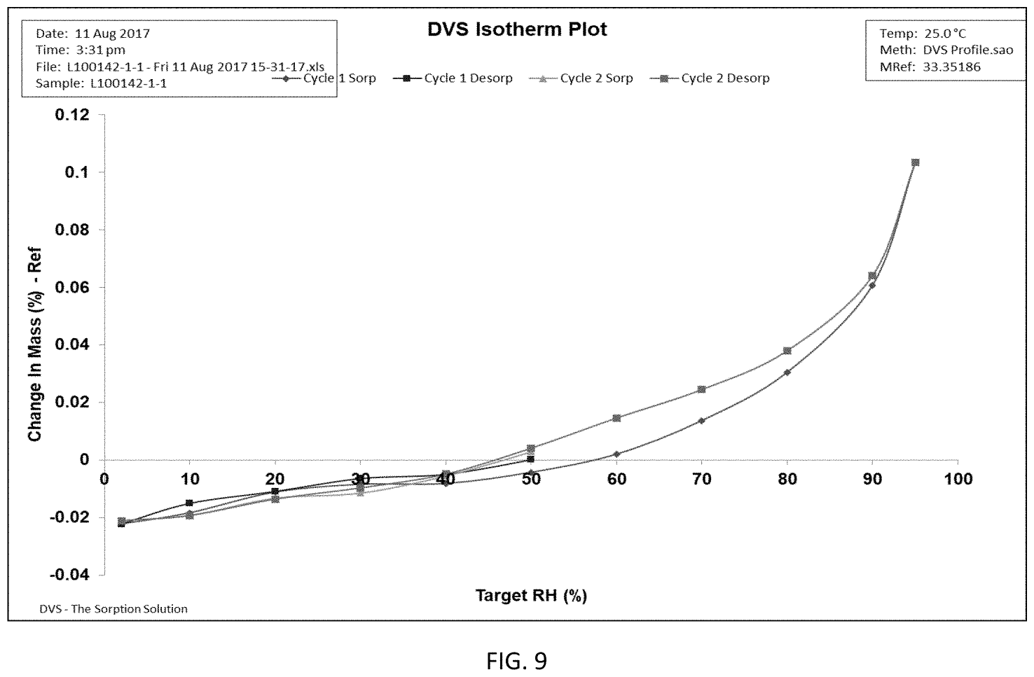

Pattern β was analyzed using DVS with two sorption/desorption cycles. The solid reversibly picked-up about 0.12% between 2-95% relative humidity at 25° C. Therefore, it is not hygroscopic (FIG. 9)

Proton Nuclear Magnetic Resonance (¹H NMR)

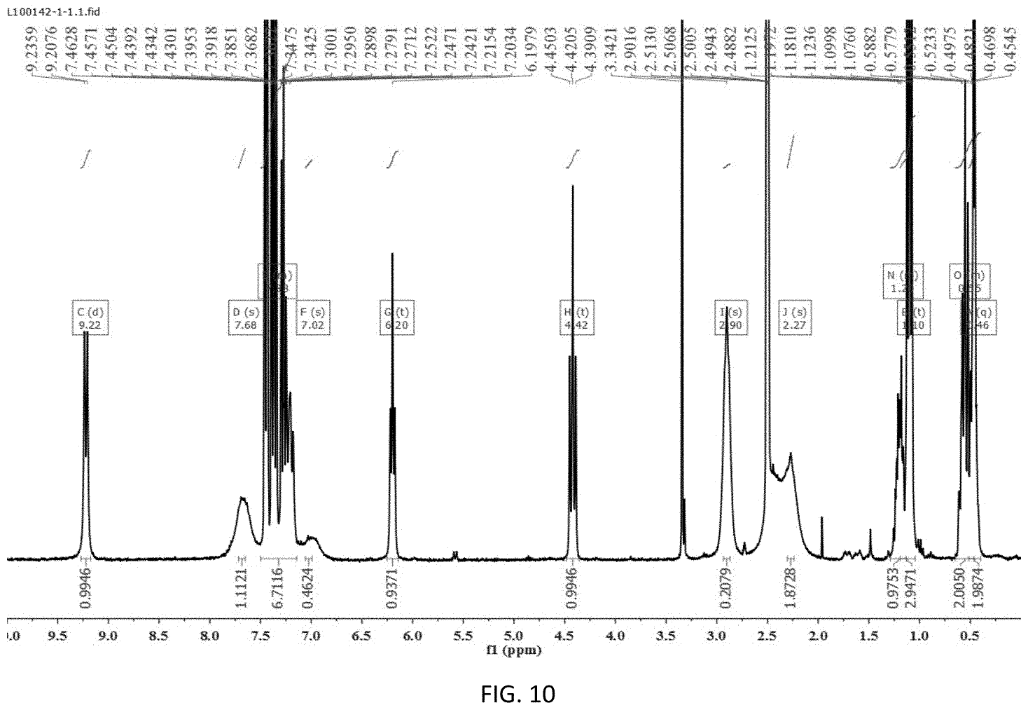

A solution ¹H NMR spectrum of SJB-01 (Lot#2223168, L100142-1-1), Pattern β, in DMSO-d₆ was obtained (FIG. 10).

Optical Microscopy

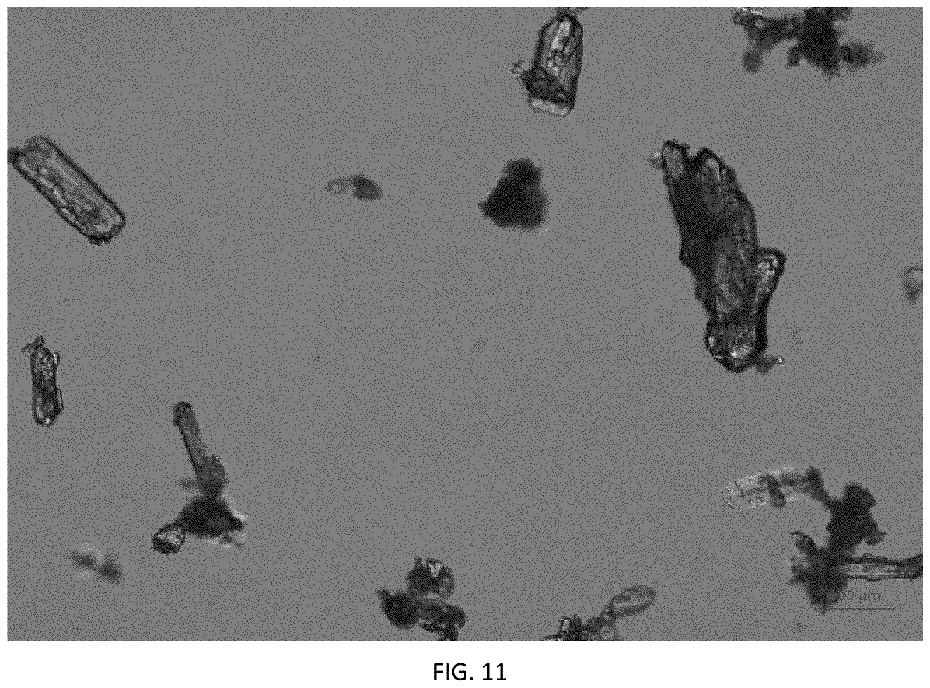

Pattern β morphology is rod-like as shown in FIG. 11.

Particle Size Analysis

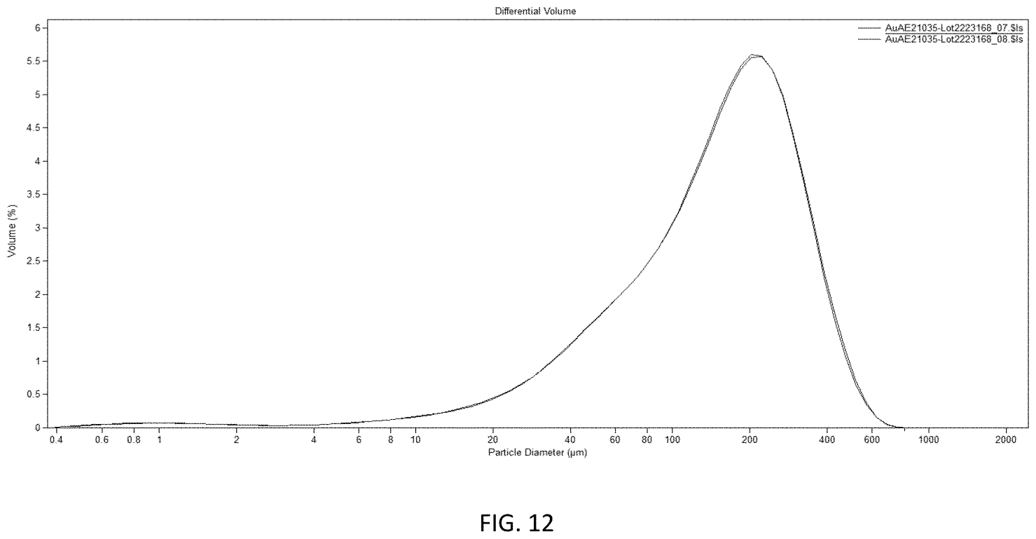

A generic method was used for particle size analysis. The results are shown in FIG. 12. The sizes are:

Median=160 μm

D[10]=40 μm

D[50]=160 μm

D[90]=336 μm

Purity by High Performance Liquid Chromatography (HPLC)

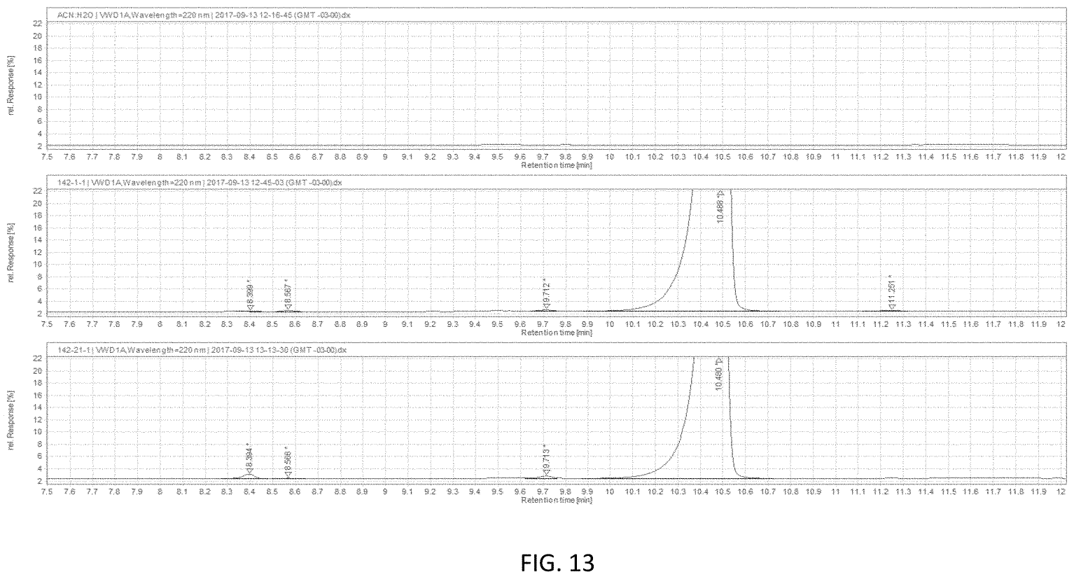

HPLC was performed on the two batches of SJB-01 to compare chemical purity and the impurity profiles using a generic method, described below in Instrumentation-High Performance Liquid Chromatography (HPLC). Both have a purity greater than 99% by peak area%, peaks with area % less than 0.03 were not included. Batch 17-12607 (L100142-21-1) had a purity of 99.2% due to a higher content of the impurities that elute at RRT 0.8 and 0.93, but do not include a peak at RRT 1.07, which is present in the batch with 99.7% purity, lot#2223168 (L100142-1-1) (FIG. 13).

TABLE 3
Peak table for HPLC chromatograms of L100142-
21-1 (SJB-01 lot#17-12607) and L100142-
1-1 (SJB-01 lot# 2223168).

| | |
|---|---|
| L100142-1-1 | L100142-21-1 |

| RRT | Area | Area % | Area | Area % |
|---|---|---|---|---|
| 0.80 | 7.71 | 0.03 | 71.62 | 0.46 |
| 0.82 | 13.55 | 0.07 | 8.19 | 0.05 |
| 0.93 | 14.99 | 0.08 | 35.01 | 0.23 |
| 1.00 | 18806.20 | 99.74 | 15379.22 | 99.26 |
| 1.07 | 16.37 | 0.08 | — | — |

Solubility Measurements and Short Term Slurry in 14 Solvents

Solubility measurements were done by gravimetric method in 14 different solvents at two temperatures (23° C. and 50° C.). About 25-35 mg solid was dispensed in 2 mL vials and 0.75 mL solvent was added to form a slurry. The slurries were then stirred for two days. The vials were centrifuged and the supernatant was collected for solubility measurement by gravimetric method. Solubility data are summarized in Table 4.

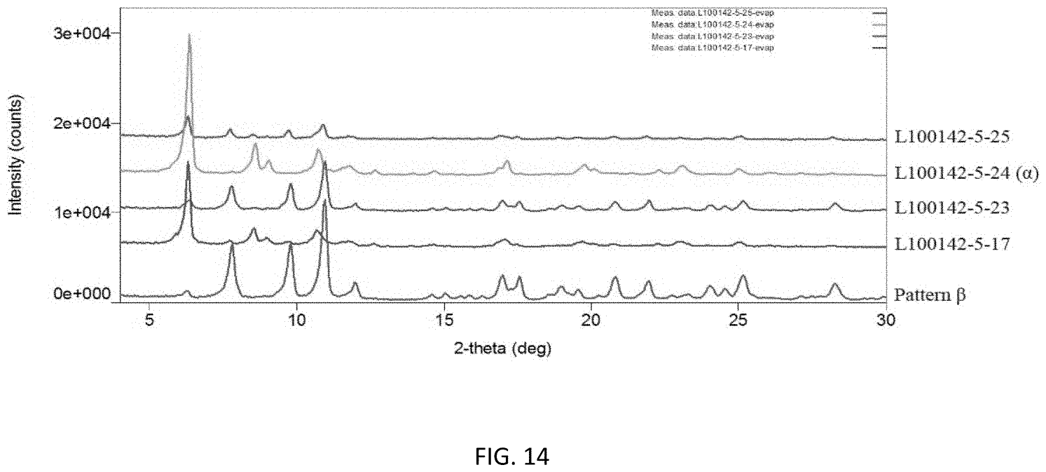

Solids from the slurry and solids after evaporation were collected for XRPD analysis. Pattern α was exhibited for solids obtained from evaporation of IPA or MIBK at 50° C., illustrated in FIG. 14. Data summarized in Table 4 and FIG. 14 illustrates XRPD patterns.

TABLE 4
Solubility of SJB-01 (L100142-1-1) in various solvent systems at 23° C. and 50° C.

| | | Solubility | | XRPD pattern of solids from | | | |
|---|---|---|---|---|---|---|---|
| | | (mg/mL) | | Slurry | | Evaporation | |
| L100142- | Solvent | 23° C. | 50° C. | 23° C. | 50° C. | 23° C. | 50° C. |
| 5-1 & 5-15 | MeOH | >76 | >76 | Diss. | Diss. | β | β |
| 5-2 & 5-16 | EtOH | >42 | >42 | Diss. | Diss. | β | β |
| 5-3 & 5-17 | IPA | 29 | 64 | β | β | β | α + traces |
| | | | | | | | of β |
| 5-4 & 5-18 | EtOAc | 42 | 67 | β | β | β | β |
| 5-5 & 5-19 | THF | >68 | >68 | Diss. | Diss. | β | β |
| 5-6 & 5-20 | Acetone | >67 | >67 | Diss. | Diss. | β | β |
| 5-7 & 5-21 | ACN | 62 | 99 | β | β | β | β |
| 5-8 & 5-22 | Dioxane | >74 | >74 | Diss. | Diss. | β | β |
| 5-9 & 5-23 | IPA:water | 27 | 60 | β | β | β | α + traces |
| | (85:15 vol) | | | | | | of β |
| 5-10 & 5-24 | MIBK | 40 | 61 | β | β | β | α |
| 5-11 & 5-25 | MtBE | 8 | 11 | β | β | β | α + traces |
| | | | | | | | of β |
| 5-12 & 5-26 | IPAc | 22 | 37 | β | β | β | β |
| 5-13 & 5-27 | Acetic acid | >72 | >72 | Diss. | Diss. | β | β |
| 5-14 & 5-28 | Water:DMSO | Nil | Nil | β | β | Insuff. | Insuff. |
| | (9:1 vol) | | | | | solid | solid |

Abbreviated Salt Screening

A stock solution in 2,2,2-Trifluoruethanol (8% w/w in TFE) of SJB-01 freebase (L100142-1-1) was prepared and 0.200 mL (equal to ˜22 mg of freebase) dispensed into vials. The counter-ion (CI) solutions were administered as ˜5% w/w solutions in EtOH, the contents were mixed and the solvents evaporated at 30° C. overnight. The solids were then further dried in a vacuum oven at 50° C. for 3 hrs. CIs chosen for screening and their pKa values are presented in Table 5.

Three solvents: EtOH, ACN and IPA:water (9:1 vol) were selected for screening. 0.22 mL (10 volumes) of the chosen solvent was added to the corresponding vial and the contents stirred for 2 days at room temperature (22-23° C.). If flowable solids developed, they were isolated and characterized by XRPD. If the solution was homogenous in phase, the vials were uncapped and the solvent evaporated. XRPD was performed on the solids recovered from the evaporated solvent.

XRPD patterns of all crystalline solids recovered from the experiments matched freebase Pattern β, indicating that a salt did not form as a solid even though it might have formed in the liquid phase due to differences in pKa. A summary of all data recorded during the screening is presented in Table 6.

TABLE 5
Counter-ions used in salt screening and associated pKa values.

| | | | Equivalents used |
|---|---|---|---|
| ID | Counter-ion | pKa | for screening |
| 1 | Benzenesulfonic acid | −2.8 | 1.1 |
| 2 | Hydrobromic acid | −9 | 1.1 |
| 3 | Hydrochloric acid | −7 | 1.1 |
| 4 | Methanesulfonic acid | −1.9 | 1.1 |
| 5 | Sulfuric acid | −3 | 1.1 |
| 6 | Toluenesulfonic acid | −2.8 | 1.1 |

TABLE 6
Data from salt screening experiments including CI and
equivalents, observations and resultant XRPD of solids.

| | SJB-01 | | Actual | After | Slurry | Slurry | XPRD |
|---|---|---|---|---|---|---|---|
| L100142-\| | (mg) | Counter-ion | CI eq. | evaporation | solvent | result | pattern |
| 3-1 | 21.2 | None | N/A | Gel | EtOH | Slurry | β |
| 3-2 | 22.1 | Benzenesulfonic acid | 1.12 | Gel | EtOH | Slurry | β |
| 3-3 | 22.5 | Hydrobromic acid | 1.07 | Gel | EtOH | Solution | β |
| 3-4 | 22.5 | Hydrochloric acid | 1.07 | Gel | EtOH | Slurry | β |
| 3-5 | 22.8 | Methanesulfonic acid | 1.13 | Gel | EtOH | Slurry | β |
| 3-6 | 22.5 | Sulfuric acid | 1.06 | Gel | EtOH | Slurry | β |
| 3-7 | 22.3 | Toluenesulfonic acid | 1.10 | Gel | ACN | Slurry | β |
| 3-8 | 22.4 | None | N/A | Gel | ACN | Slurry | β |
| 3-9 | 22.4 | Benzenesulfonic acid | 1.11 | Gel | ACN | Solution | Gel |
| 3-10 | 22.4 | Hydrobromic acid | 1.13 | Gel | ACN | Solution | Gel |
| l3-11 | 22.5 | Hydrochloric acid | 1.10 | Gel | ACN | Solution | β |
| 3-12 | 21.7 | Methanesulfonic acid | 1.15 | Gel | ACN | Solution | β |
| 3-13 | 22.0 | Sulfuric acid | 1.10 | Gel | ACN | Solution | β |
| 3-14 | 21.5 | Toluenesulfonic acid | 1.15 | Gel | ACN | Solution | β |
| 3-15 | 22.2 | None | N/A | Gel | IPA:water 9:1 | Slurry | β |
| 3-16 | 21.5 | Benzenesulfonic acid | 1.11 | Gel | IPA:water 9:1 | Slurry | β |
| 3-17 | 22.4 | Hydrobromic acid | 1.16 | Gel | IPA:water 9:1 | Slurry | β |
| 3-18 | 21.3 | Hydrochloric acid | 1.09 | Solid on | IPA:water 9:1 | Slurry | β |
| | | | | the wall | | | |
| 3-19 | 22.2 | Methanesulfonic acid | 1.17 | Gel | IPA:water 9:1 | Slurry | β |
| 3-20 | 22.3 | Sulfuric acid | 1.11 | Gel | IPA:water 9:1 | Slurry | β |
| 3-21 | 22.1 | Toluenesulfonic acid | 1.15 | Gel | IPA:water 9:1 | Slurry | β |

Polymorph Screening

Slurry Experiments of Crystalline Pattern 13 in Additional Solvents

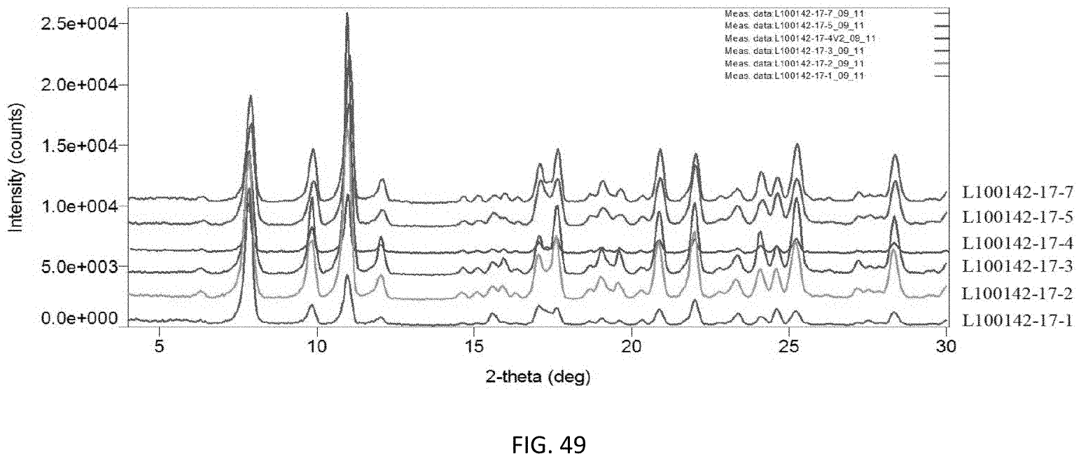

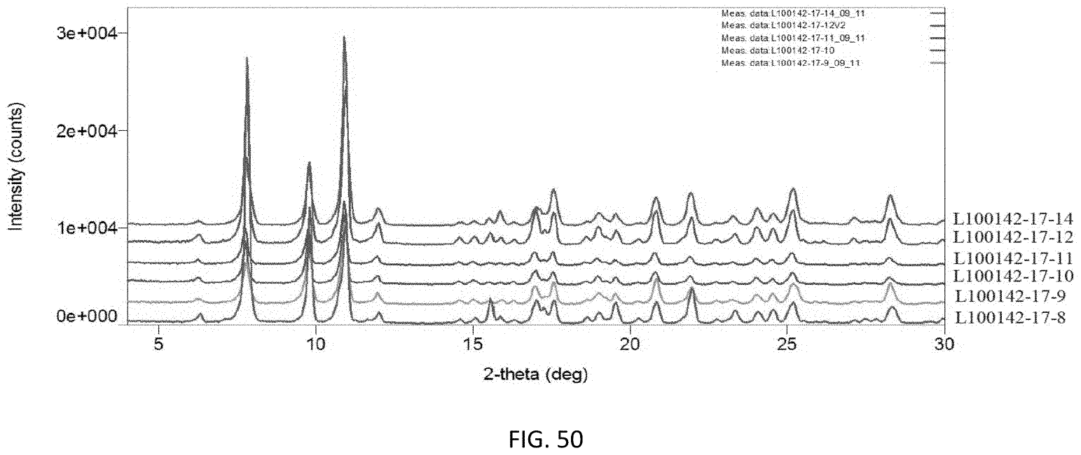

Slurries of SJB-01 freebase (Pattern β, L100142-1-1) in 0.3 mL of one of 7 solvents were made at room temperature (22-23° C.) and 50° C. and stirred. See Table 7 for a summary of details and FIGS. 49-50 for XRPD patterns. Despite the addition of roughly 90 mg to benzyl alcohol at room temperature (22-23° C.), a slurry never formed, all solids dissolved quickly and results from this solvent at both room temperature (22-23° C.) and 50° C. are not applicable. All XRPD patterns exhibited by recovered solids after a couple days (experiment dependent) matched Pattern β.

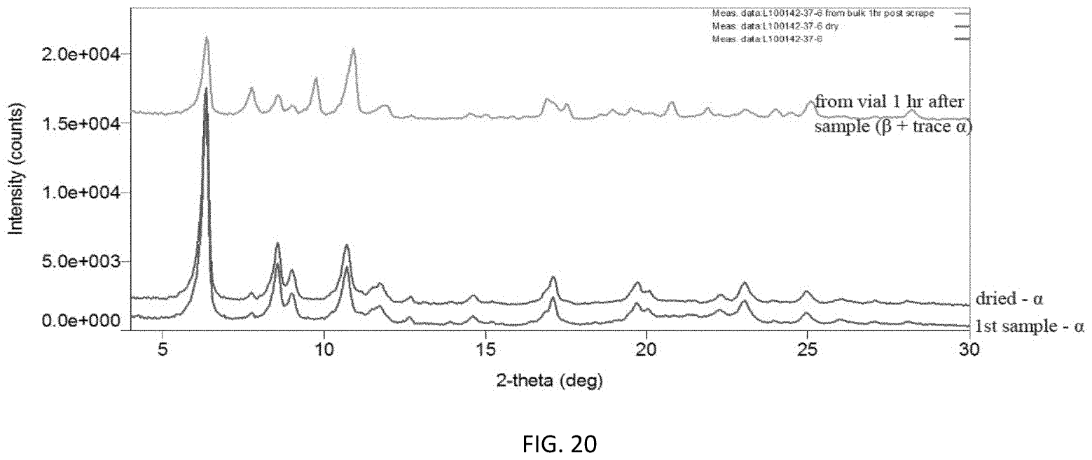

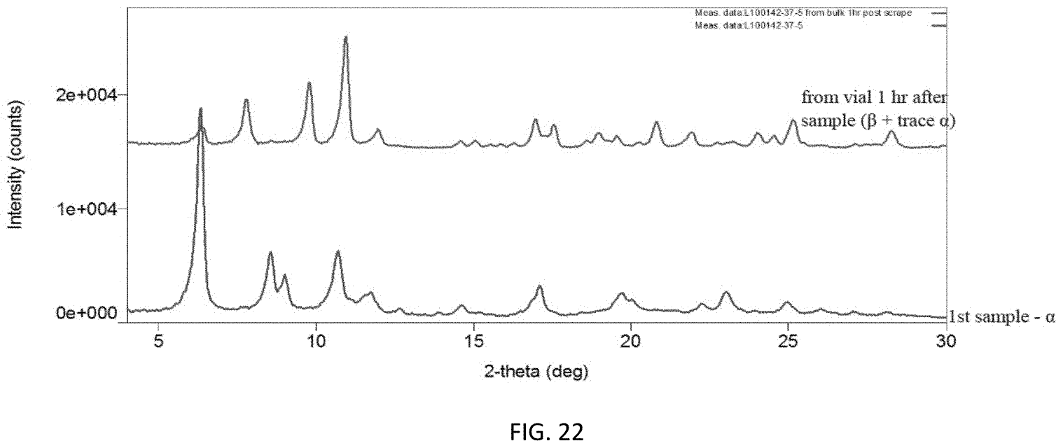

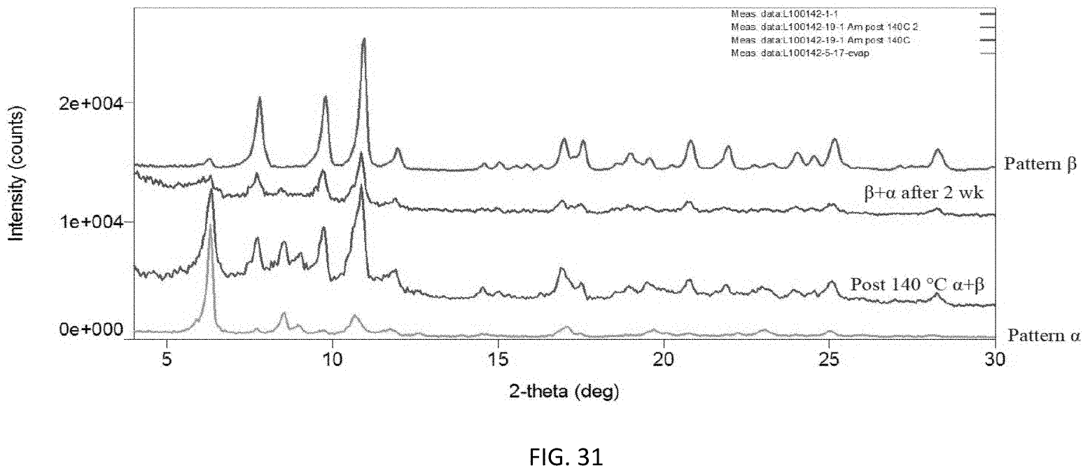

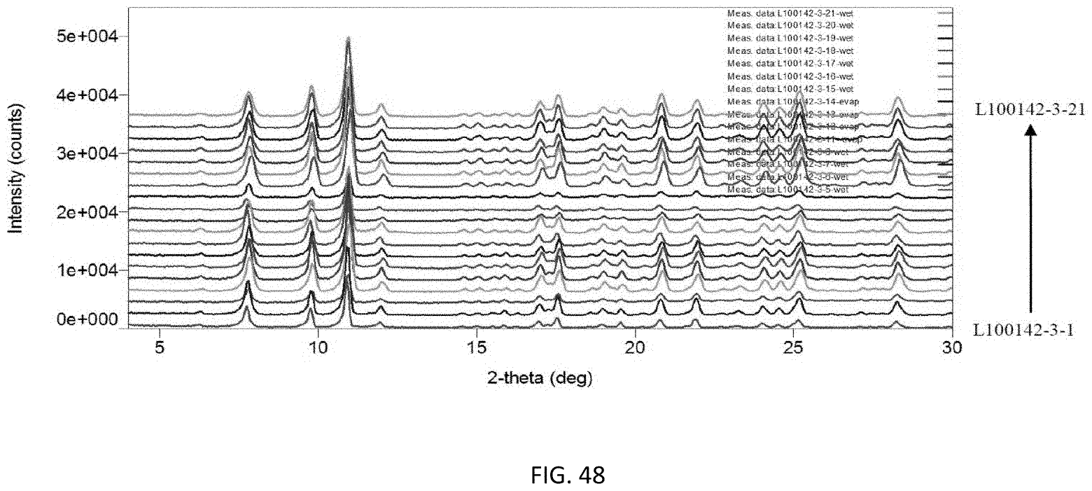

TABLE 7
Summary of data for slurry experiments of SJB-01
(L100142-1-1) Pattern β in 7 solvents. Corresponding
XRPD patterns are illustrated in FIGS. 4-50.

| | | | Slurry | XRPD of |
|---|---|---|---|---|
| L100142- | Solvent | Condition | time | slurried solids |
| 17-1 | Toluene | RT | 3 d | β |
| 17-2 | MEK | | 2 d | β |
| 17-3 | Nitromethane | | 2 d | β |
| 17-4 | MeOAc | | 3 d | β |
| 17-5 | Anisole | | 2 d | β |
| 17-6 | Benzyl alcohol | | N/A | N/A |
| 17-7 | 1-BuOH | | 3 d | β |
| 17-8 | Toluene | 50° C. | 2 d | β |
| 17-9 | MEK | | 2 d | β |
| 17-10 | Nitromethane | | 2 d | β |
| 17-11 | MeOAc | | 2 d | β |
| 17-12 | Anisole | | 2 d | β |
| 17-13 | Benzyl alcohol | | N/A | N/A |
| 17-14 | 1-BuOH | | 2 d | β |

Cooling Crystallization

Fast and Slow Cooling with SJB-01 lot L100142-1-1

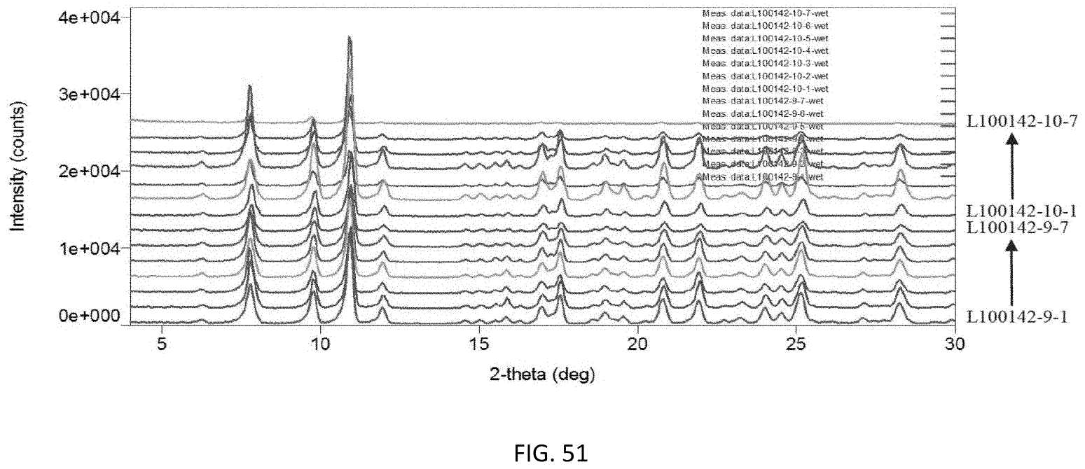

Fast and slow cooling experiments were conducted by forming saturated solutions with about 25 mg of SJB-01 (L100142-1-1) in the chosen solvent at 50° C. In fast cooling experiments, solutions were cooled quickly to 0° C. by placing the vial in an ice bath. In the slow cooling experiments, the solutions were cooled to room temperature (22-23° C.) over 3 hours. The resultant solids were all Pattern β, confirmed by XRPD (FIG. 51). Experimental details and results are summarized in Table 8.

TABLE 8
Results for fast and slow cooling crystallization experiments with
SJB-01 (L100142-1-1). XRPD patterns are illustrated in FIG. 51.

| | | Solvent | XRPD pattern | |
|---|---|---|---|---|
| L100142- | Solvent | vol | Fast | Slow |
| 9-1 & 10-1 | IPA:water(85:15) | 20 | β | β |
| 9-2 & 10-2 | MeOH:water(85:15) | 20 | β | β precipitated |
| | | | | O/N |
| 9-3 & 10-3 | MIBK | 20 | β | β |
| 9-4 & 10-4 | IPA | 15 | β | β |
| 9-5 & 10-5 | EtOAc | 15 | β | β |
| 9-6 & 10-6 | IPAc | 30 | β | β |
| 9-7 & 10-7 | Acetone:Hep- | 20 | β | β (small solid |
| | tane(2:1) | | | weight, low yield), |
| | | | | precipitated O/N |

Fast and Slow Cooling with SJB-01 lot L100142-21-1

The cooling crystallization screening experiment was repeated with a second batch of SJB-01, lot 17-12607 (L100142-21-1). A smaller scale of ˜17 mg was used, maintaining a proportional volume of the solvent. No changes in results were observed, the XRPD pattern of all solids recovered match Pattern β. Data are summarized in Table 9.

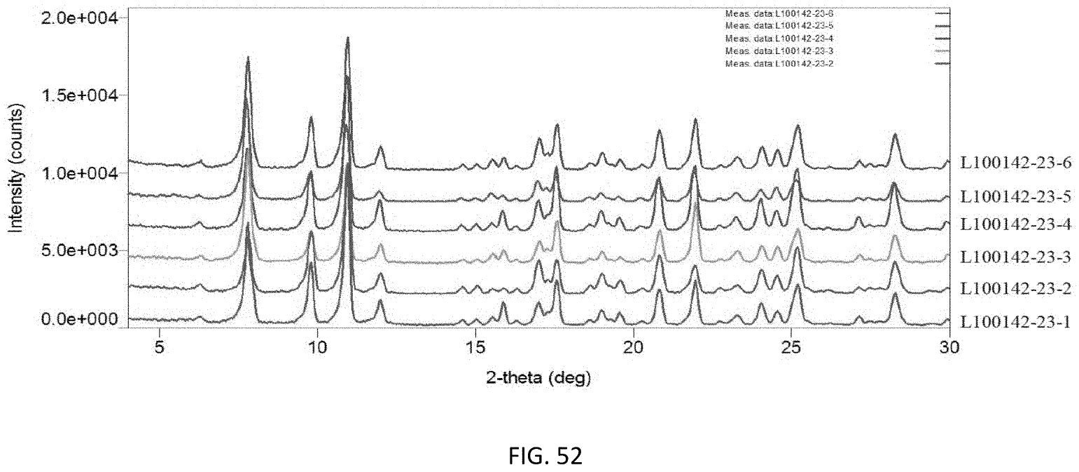

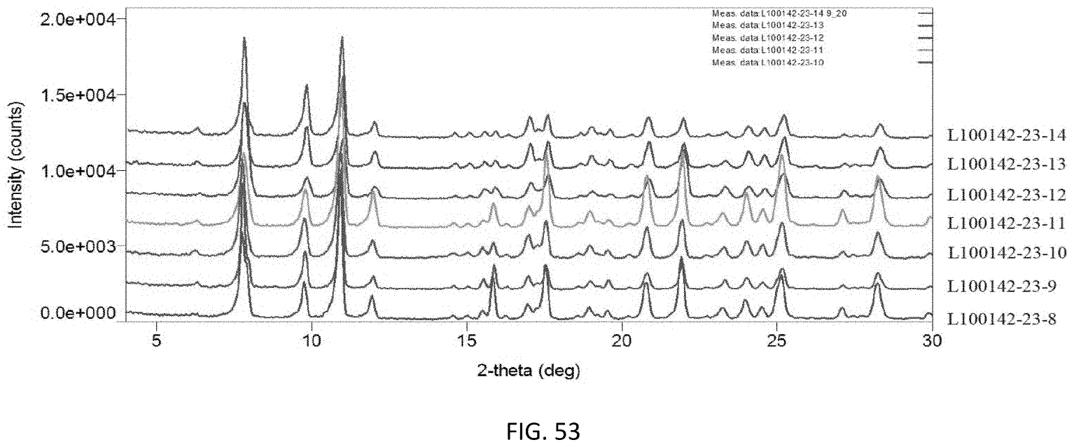

TABLE 9
Summary of data for cooling crystallization experiments with
SJB-01 lot 17-12607. XRPD are illustrated in FIGS. 52-53.

| | | Crystal- | | |
|---|---|---|---|---|
| | | lization | Cooling | |
| L110142- | Solvent | temperature | method | XRPD |
| 23-1 | IPA:water (85:15 vol.) | 0° C. | Slow | β |
| 23-2 | MeOH:water (85:15 vol.) | RT | cooling | β |
| 23-3 | MIBK | 0° C. | (5° C./hr) | β |
| 23-4 | IPA | 5° C. | | β |
| 23-5 | EtOAc | RT | | β |
| 23-6 | IPAc | 7.5° C. | | β |
| 23-7 | Acetone:heptane | 0° C. | | insuff. |
| | (2:1 vol.) | | | solid |
| 23-8 | IPA:water (85:15 vol.) | 0° C. | Fast | β |
| 23-9 | MeOH:water (85:15 vol.) | 0° C. | cooling | β |
| 23-10 | MIBK | 0° C. | | β |
| 23-11 | IPA | 0° C. | | β |
| 23-12 | EtOAc | 0° C. | | β |
| 23-13 | IPAc | 0° C. | | β |
| 23-14 | Acetone:heptane | −20 | | β |
| | (2:1 vol.) | | | |

Stagnant Cooling Crystallizations

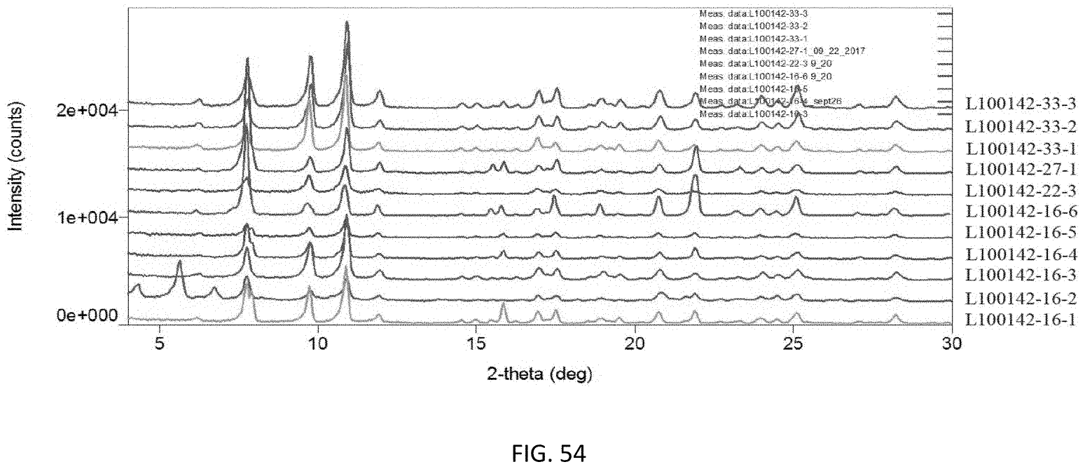

Saturated solutions of SJB-01 were made in a chosen solvent at a chosen initial temperature. These saturated solutions were then set at a final temperature and left unstirred. Solids that precipitated were collected and analyzed by XRPD. Conditions for the various experiments are summarized in Table 10 and XRPD patterns are illustrated in FIG. 54.

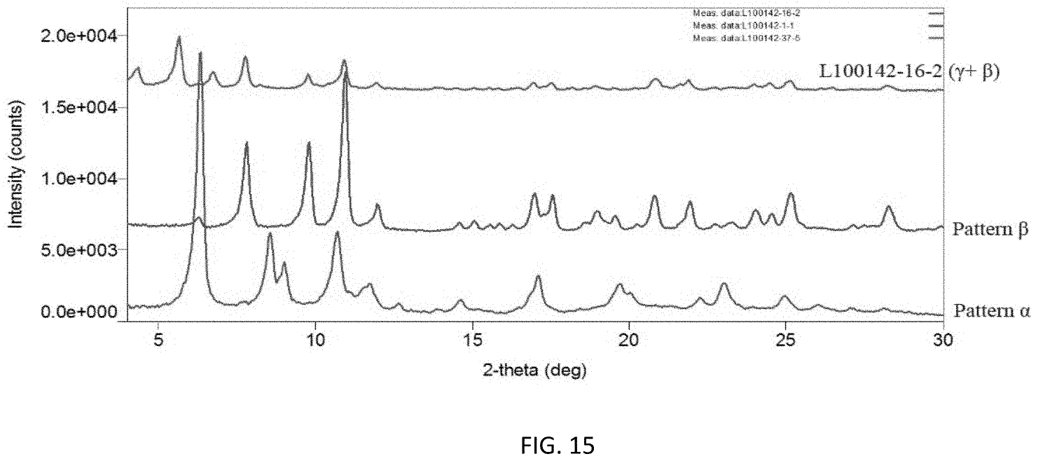

Saturated solutions of SJB-01 freebase (L100142-1-1) were prepared in IPA, EtOAc, ACN, MIBK, MtBE and IPAc at room temperature (22-23° C.) and set at −20° C. After one week, dendritic white solids appeared on the walls of vial L100142-16-2 (EtOAc). These were collected and showed new unique reflections in the XRPD pattern, along with all the expected peaks for Pattern β FIG. 15). Clear-white block crystals developed in the remainder of the vials and these exhibited Pattern β by XRPD analysis.

A saturated solution of SJB-01 (L100142-21-1) in EtOAc was made at room temperature (22-23° C.) then set at −20° C. Blocky, clear-white crystals were collected a week later (L100142-22-3), and exhibited Pattern β when analyzed by XRPD.

A saturated solution of SJB-01 (L100142-1-1) in EtOAc was made at room temperature (22-23° C.) then set at −20° C. After 3 hrs, the still clear solution was seeded with L100142-16-2, which showed a mixture of Pattern γ+β. Blocky, clear-white crystals were collected the next day (L100142-27-1), and exhibited Pattern β when analyzed by XRPD.

A saturated solution of SJB-01 (L100142-1-1) in IPA and 1-BuOH was made at 63° C. then set at room temperature (22-23° C.). An additional vial of saturated IPA was made and set at −20° C. The following day crystalline solids were collected from all three vials and all exhibited Pattern β when analyzed by XRPD.

TABLE 10
Summary of data for stagnant cooling crystallizations. XRPD patterns are illustrated in FIG. 54.

| | | | Initial | Final T | | |
|---|---|---|---|---|---|---|
| L100142- | SJB-01 lot | Solvent | T (° C.) | (° C.) | Comments | XRPD |
| 16-1 | L100142-1-1 | IPA | 22-23 | −20 | Blocky clear-white crystals collected | β |
| | | | | | after 2 weeks | |
| 16-2 | L100142-1-1 | EtOAc | 22-23 | −20 | Dendritic white solids collected after | γ + β |
| | | | | | 1 week | |
| 16-3 | L100142-1-1 | ACN | 22-23 | −20 | Blocky clear-white crystals collected | β |
| | | | | | after 1 week | |
| 16-4 | L100142-1-1 | MIBK | 22-23 | −20 | Blocky clear-white crystals collected | β |
| | | | | | after 2 weeks | |
| 16-5 | L100142-1-1 | MtBE | 22-23 | −20 | Blocky clear-white crystals collected | β |
| | | | | | after 2 weeks | |
| 16-6 | L100142-1-1 | IPAc | 22-23 | −20 | Blocky clear-white crystals collected | β |
| | | | | | after 2 weeks | |
| 22-3 | L100142-21-1 | EtOAc | 22-23 | −20 | Blocky clear-white crystals collected | β |
| | | | | | after 2 weeks | |
| 27-1 | L100142-1-1 | EtOAc | 22-23 | −20 | Seeded with L100142-16-2. Blocky | β |
| | | | | | clear-white crystals collected next day | |
| 33-1 | L100142-1-1 | IPA | 63 | 22-23 | Blocky clear-white crystals collected | β |
| | | | | | next day | |
| 33-2 | L100142-1-1 | 1-BuOH | 63 | 22-23 | Blocky clear-white crystals collected | β |
| | | | | | next day | |
| 33-3 | L100142-1-1 | IPA | 63 | −20 | Blocky clear-white crystals collected | β |
| | | | | | next day | |

Anti-Solvent Crystallization

About 25 mg of SJB-01 (L100142-1-1) was weighed into 2 mL vials and dissolved in the chosen solvent. For direct addition, the anti-solvent was added to the solutions dropwise until precipitation was observed. For reverse experiments, an equal volume of anti-solvent was prepared in a separate vial and the SJB-01 solution was added to anti-solvent all at once. The experiment was performed at room temperature (22-23° C.). The results are presented in Table 11.

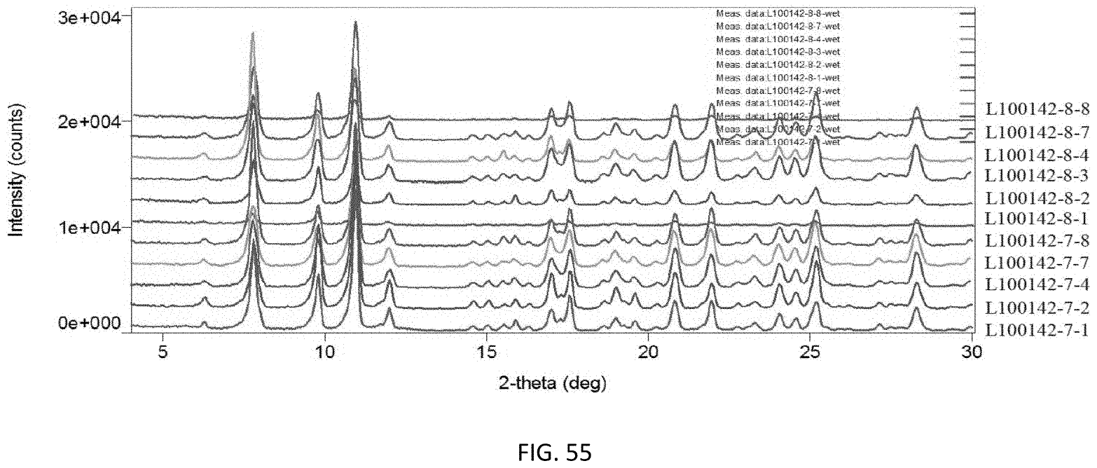

No precipitation was observed in acetone or acetic acid with the use of heptane as the antisolvent in direct or reverse experiments. Oiling occurred in THF with direct addition of water. The XPRD patterns of all other solids were Pattern β. See FIG. 55 for XRPD patterns.

TABLE 11
Details and results of the anti-solvent crystallization experiments.
Corresponding XRPD patterns are illustrated in FIG. 55.

| | | | | Anti-solvent | | | | XRPD | |
|---|---|---|---|---|---|---|---|---|---|
| | | Solvent | Anti- | volume | | Comments | | pattern | |
| L100142- | Solvent | volume | solvent | Direct | Reverse | Direct | Reverse | Direct | Reverse |
| 7-1 & | MeOH | 15 | Water | 2 | 15 | Precipitated | Precipitated | β | β |
| 8-1 | | | | | | | | | |
| 7-2 & | Acetic | 10 | Water | 8 | 10 | Precipitated | Precipitated | β | β |
| 8-2 | acid | | | | | | | | |
| 7-3 & | THF | 10 | Water | 6 | 10 | oil | precipitated | NA | β |
| 8-3 | | | | | | | after O/N | | |
| 7-4 & | Acetone | 15 | Water | 6 | 15 | Precipitated | Precipitated | β | β |
| 8-4 | | | | | | | | | |
| 7-5 & | EtOH | 20 | Heptane | 35 | 20 | no | no | NA | NA |
| 8-5 | | | | | | precipitate | precipitate | | |
| 7-6 & | Acetic | 10 | Heptane | 38 | 10 | no | no | NA | NA |
| 8-6 | acid | | | | | precipitate | precipitate | | |
| 7-7 & | THF | 10 | Heptane | 12 | 10 | Precipitated | Precipitated | β | β |
| 8-7 | | | | | | | | | |
| 7-8 & | Acetone | 15 | Heptane | 38 | 15 | Precipitated | Precipitated | β | β |
| 8-8 | | | | | | | | | |

Evaporative Crystallization

Flash Evaporation

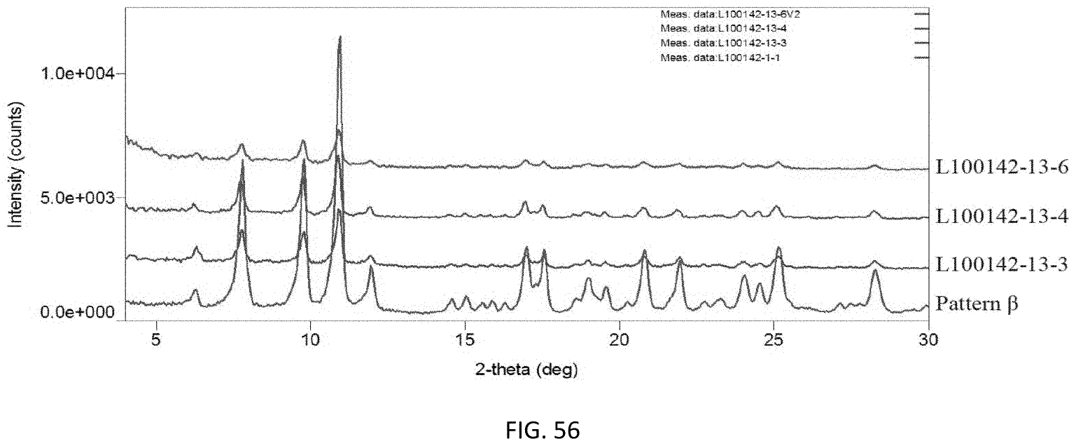

Solutions of SJB-01 freebase (L100142-1-1) in 6 solvents (MeOH, EtOH, THF, acetone, CAN and MIBK) were prepared at 50° C. These were added dropwise to microscope slides at 120° C., resulting in rapid evaporation of the solvent and a clear-colorless gel remained on the slide. Viscous bubbling occurred during the evaporation of acetone and this was disturbed by popping the bubbles with a glass pipette, yielding some opaque white solids on the slide. The slides were removed from the heat as soon as evaporation of the solvent was completed. Transformation within the gel to opaque white solids was observed on two more slides (gels from THF and MIBK) over the next 2 hr at room temperature. Slides showing white solids were scraped and analyzed by XRPD. All were less crystalline Pattern β FIG. 56). The slides on which clear gel remained were scratched in an attempt to initiate nucleation, but no conversion was observed up to 1 month later. Data for these experiments are summarized in Table 12.

TABLE 12
Details for flash evaporation experiments including solution
solvents and volumes, and resultant XRPD pattern of solids
after evaporation. XRPD are illustrated in FIG. 56.

| L100142- | Solvent | vol. | Comments | XRPD |
|---|---|---|---|---|
| 13-1 | MeOH | 10 | Clear gel. Scratched to initiate | — |
| | | | nucleation. | |
| 13-2 | EtOH | 10 | Clear gel, some “fogging” of | — |
| | | | the gel after ~2 hr. Scratched to | |
| | | | initiate nucleation. | |
| 13-3 | THF | 8 | Clear gel, substantial conversion to | β |
| | | | white solids over 2 hrs. | |
| 13-4 | Acetone | 6 | Disturbed with pipette while | β |
| | | | evaporating, developed white | |
| | | | solids. | |
| 13-5 | ACN | 6 | Clear gel. Scratched to initiate | — |
| | | | nucleation. | |
| 13-6 | MIBK | 14 | Clear gel, substantial conversion to | β |
| | | | white solids over 2 hrs. | |

Rotary Evaporation

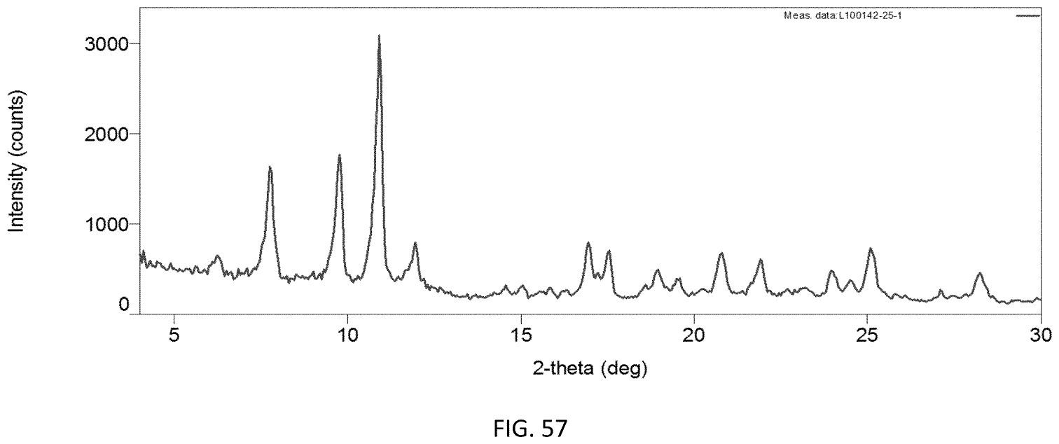

About 25 mg of SJB-01 was dissolved in 1.5 mL of MIBK and the solvent was evaporated on a rotary evaporator using a hot water bath at 70° C. The resultant solid was clear and colorless, stuck to the walls of the vial. Over 24 hrs, L100142-25-1 (used L100142-1-1 as input) transformed to an opaque white solid which exhibited Pattern β by XRPD analysis (FIG. 57). Opaque white regions grew within the amorphous glass for sample L100142-25-2 (used L100142-21-1 as input).

TABLE 13
Summary of data for rotary evaporation of solutions of SJB-01 in MIBK.

| | SJB-01 | | Evaporation | | |
|---|---|---|---|---|---|
| L100142- | lot | Solvent | T (° C.) | Comments | XRPD |
| 25-1 | L100142- | MIBK | 70 | Clear gel initially. Most | β |
| | 1-1 | | | converted to opaque white | |
| | | | | solid overnight. | |
| 25-2 | L100142- | MIBK | 70 | Clear gel initially. Small | β |
| | 21-1 | | | amount of opaque white solid | |
| | | | | grew within gel overnight. | |

Stirred Evaporation

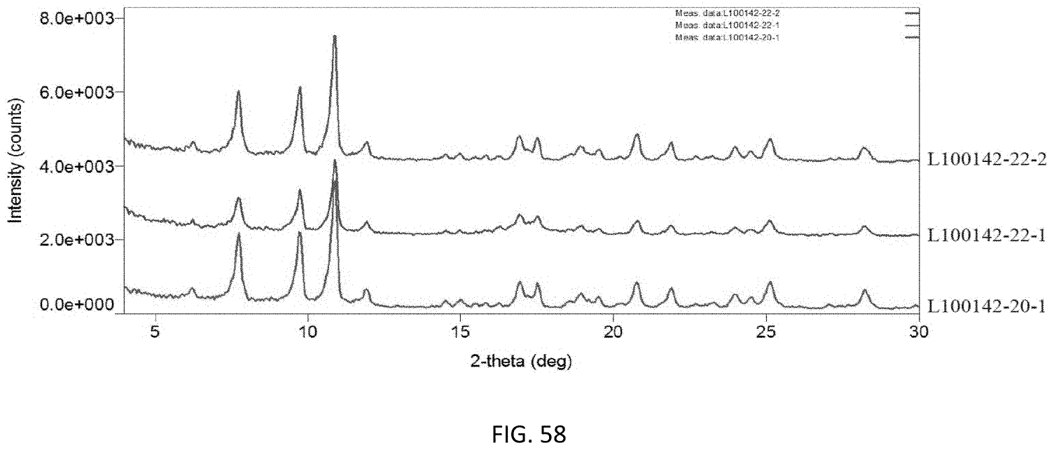

With the precedents of Pattern α having been obtained from evaporation of IPA and MIBK at 50° C., experiments aimed at generating Pattern α were performed by dissolving SJB-01 in IPA or MIBK to form near-saturated solutions at 50° C., then the caps removed to allow the solvent to evaporate overnight. The solutions were allowed to continue stirring during evaporation. All experiments yielded Pattern β. Table 14 provides a summary of the conditions and results of the experiments, and XRPD are illustrated in FIG. 58.

TABLE 14
Summary of data for stirred evaporation experiments.
XRPD patterns are illustrated in FIG. 58.

| | | | Evaporation | XRPD |
|---|---|---|---|---|
| L100142- | SJB-01 lot | Solvent | T (° C.) | pattern |
| 20-1 | L100142-1-1 | MIBK | 50 | β |
| 22-1 | L100142-21-1 | IPA | 50 | β |
| 22-2 | L100142-21-1 | MIBK | 50 | β |

Stagnant Evaporation

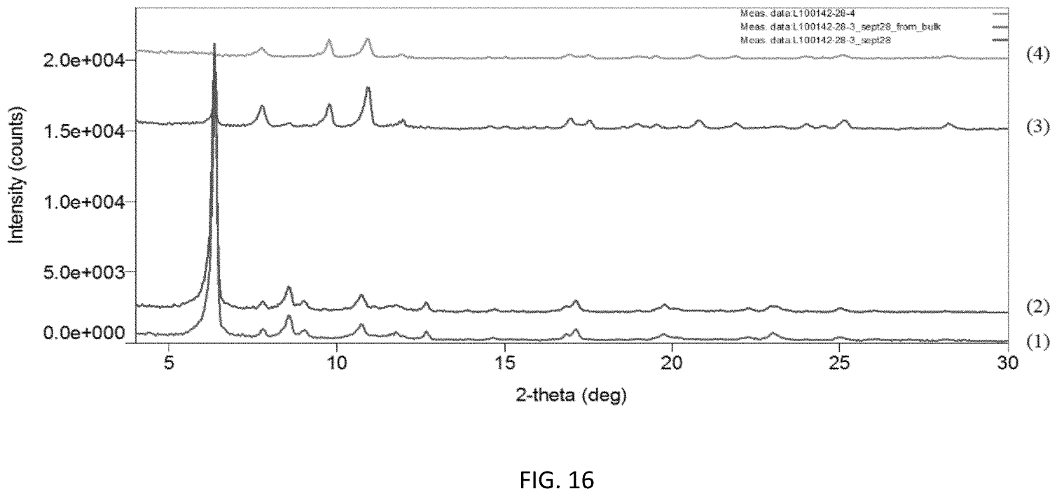

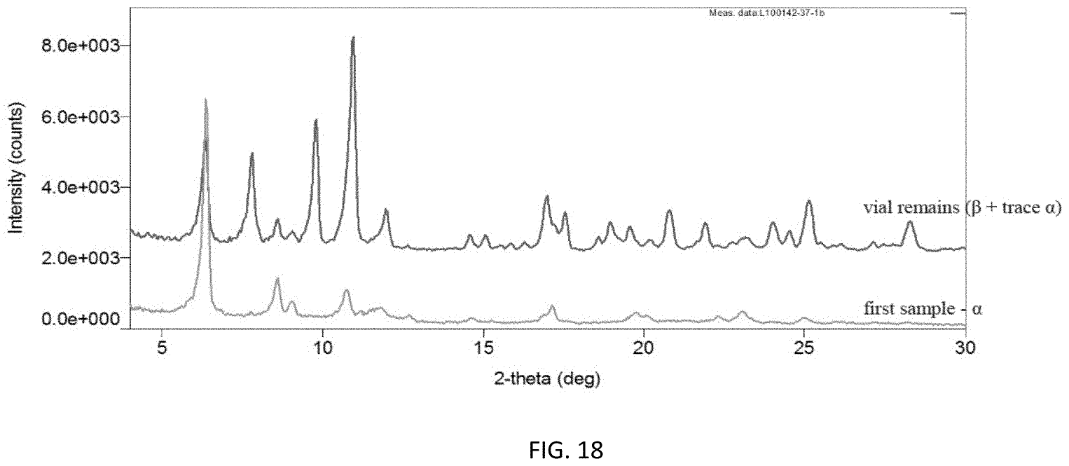

With the precedents of Pattern α having been obtained from evaporation of IPA and MIBK at 50° C., experiments aimed at generating Pattern α were performed by dissolving SJB-01 (L100142-1-1) in MIBK, IPA, 1-PA or 1-BuOH and allowing the solvent to evaporate at a chosen temperature unstirred. Conditions and results are summarized in Table 15 and XRPD are illustrated in FIG. 16, FIGS. 18-20, and FIG. 59. In cases where Pattern α was obtained, the sample taken for XRPD was stable on the XRD plate over 24 hrs, but the solids that were left in the vial from which the sample was taken transformed to become primarily Pattern β with only traces of a, even as soon as 1 hr later.

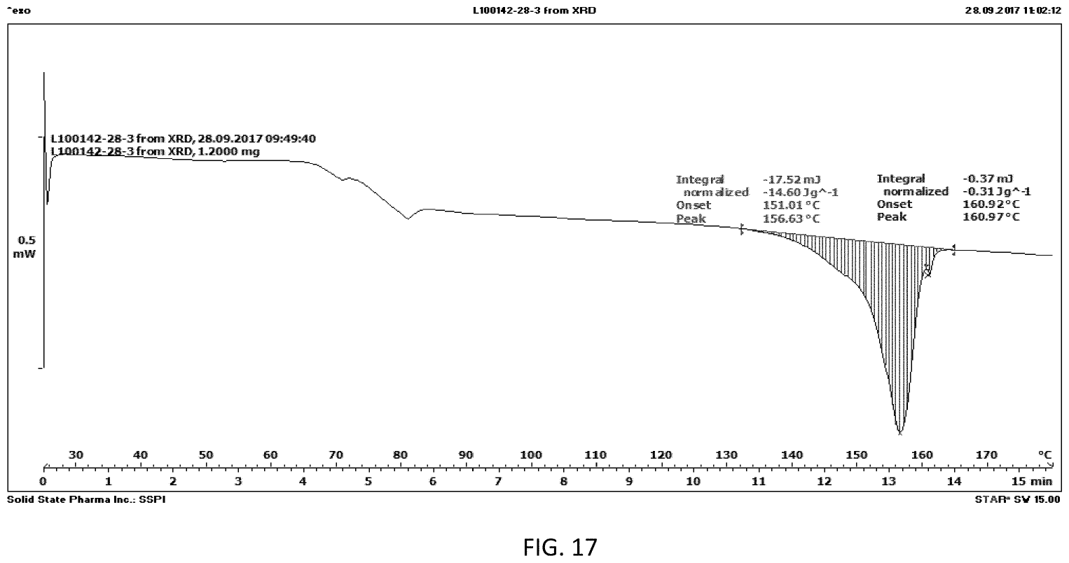

Saturated solutions of SJB-01 in MIBK and IPA were prepared and allowed to evaporate at 50° C. The solids from MIBK (L100142-28-4) yielded Pattern β and the solids from IPA (L100142-28-3) showed Pattern α (FIG. 10) by XRPD. The same XRD plate still showed Pattern α the next day, but the solids that remained in the vial had almost entirely converted to Pattern β. DSC of the XRD sample, Pattern α (L100142-28-3), shows a major endotherm with an onset temperature of 151.0 and peak of 156.6° C. as well as a minor endotherm with a calculated onset and peak of 160.9° C. (FIG. 17). These two endotherms correspond with the melting points of SJB-01 Patterns α and β respectively.

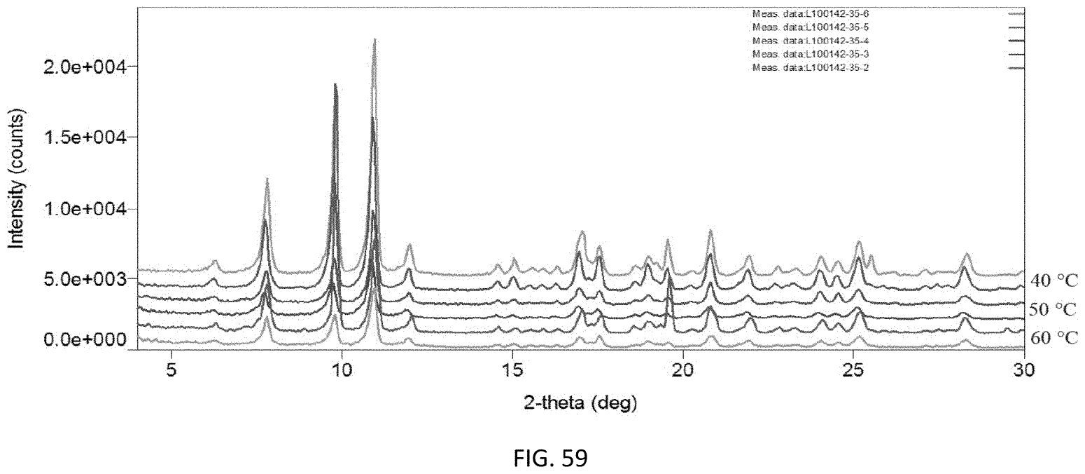

A saturated solution of SJB-01 in IPA was made and separated into 6 vials. Two were set at each temperature of 40° C. (L100142-35-1 and L100142-35-2), 50° C. (L100142-35-3 and L100142-35-4) and 60° C. (L100142-35-5 and L100142-35-6) for the IPA to evaporate unstirred. At 40 and 50° C., Pattern β was recovered. At 60° C., a gel formed, and Pattern β grew within the gel for both vials. XRPD patterns are presented in FIG. 59.

Evaporation from 1-propanol and 1-BuOH at 90° C. yielded gels. From the gel that developed from evaporation of 1-propanol (L100142-37-1), Pattern α grew over time at 50° C. After sampling for XRPD analysis, the solids that remained in the vial transformed into Pattern β with traces of a (FIG. 18). 1-BuOH (L100142-37-2) remained a gel at 50° C. after 24 hr.

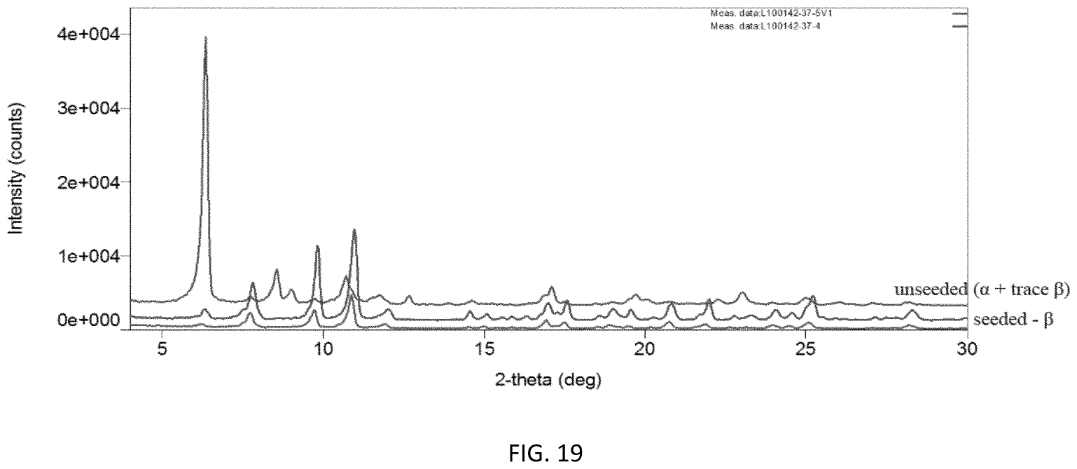

Two vials of gel formed by evaporating a solution of SJB-01 in 1-propanol at 90° C. were set at 50° C. and seeded with Pattern α. The gels converted to Pattern β overnight (L100142-37-3 and L100142-37-4). A second attempt to generate Pattern α without seeding by evaporation of 1-propanol at 90° C. yielded a gel from which about ⅓rd transformed into Pattern α (+trace β) at the time of sampling (L100142-37-5). These XRPD patterns are presented in FIG. 19.

Two vials of SJB-01 dissolved in 1-propanol were set at 90° C. The 1-propanol evaporated to yield a gel within which nucleation and propagation of a crystalline form began before transferring to 50° C. and allowing the bulk of the gel to crystallize. Samples from both vials analyzed by XRPD show Pattern α. Solids in the vials transformed into primarily Pattern β within an hour, the solid on the XRD plates remained a over this time period.

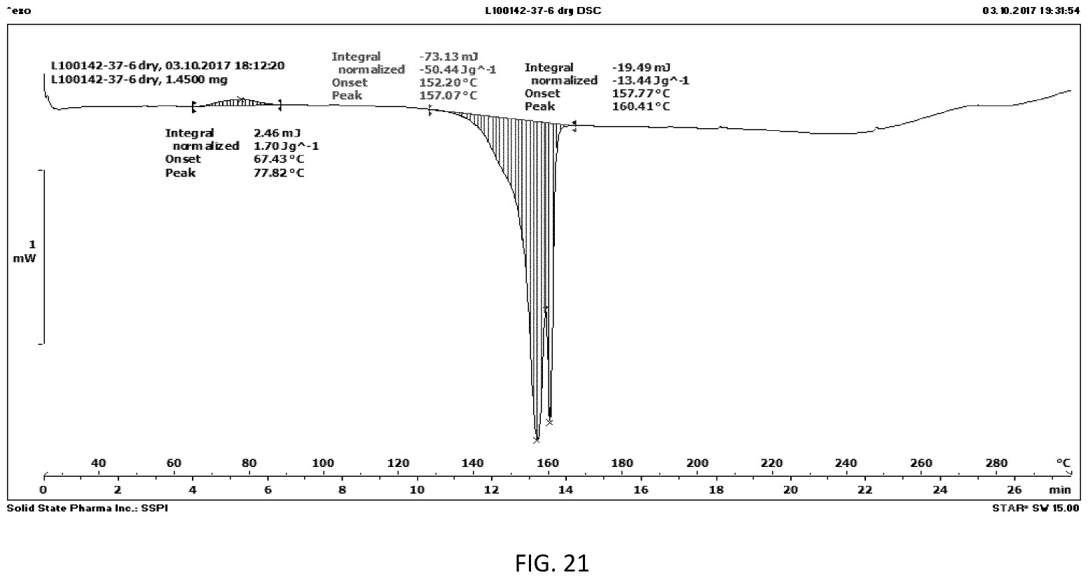

A sample from the XRD plate of L100142-37-6 was used for DSC, and shows two melting endotherms at temperatures characteristic for melting of Patterns a and β FIG. 21).

TABLE 15
Summary of data for stagnant evaporation experiments. XRPD
are illustrated in FIG. 16, FIGS. 18-20, and FIG. 59.

| | | Evaporation | | XRPD |
|---|---|---|---|---|
| L100142- | Solvent | T (° C.) | Comments | pattern |
| 28-3 | IPA | 50 | Heated solution to 60° C. to ensure no solids | α |
| | | | before evaporating at 50° C. | |
| 28-4 | MIBK | 50 | Heated solution to 60° C. to ensure no solids | β |
| | | | before evaporating at 50° C. | |
| 32-1 | IPA | 50 | Used saturated solution | β |
| 32-2 | MIBK | 50 | Used saturated solution | β |
| 35-1 | IPA | 40 | Solution saturated at 50° C. before evaporation | β |
| 35-2 | IPA | 40 | Solution saturated at 50° C. before evaporation | β |
| 35-3 | IPA | 50 | Used saturated solution | β |
| 35-4 | IPA | 50 | Used saturated solution | β |
| 35-5 | IPA | 60 | Solution saturated at 50° C. before evaporation. | β |
| | | | Evaporation gives gel which transforms to | |
| | | | crystalline solid | |
| 35-6 | IPA | 60 | Solution saturated at 50° C. before evaporation. | β |
| | | | Evaporation gives gel which transforms to | |
| | | | crystalline solid | |
| 37-1 | 1-PrOH | 90 | Evaporation yields gel. Set at 50° C., gel → | α + β |
| | | | crystalline solid | |
| 37-2 | 1-BuOH | 90 | Evaporation yields gel. Set at 50° C., gel → | gel |
| | | | crystalline solid | |
| 37-3 | 1-PrOH | 100 | Evaporation yields gel. Set at 50° C. and seeded | β |
| | | | with 37-1 | |
| 37-4 | 1-PrOH | 90 | Evaporation yields gel. Set at 50° C. and seeded | β |
| | | | with 37-1 | |
| 37-5 | 1-PrOH | 90 | Evaporation yields gel. Set at 50° C., gel → | α |
| | | | crystalline solid | |
| 37-6 | 1-PrOH | 90 | Evaporation yields gel. Set at 50° C., gel → | α |
| | | | crystalline solid | |

Dry and Solvent Drop Milling

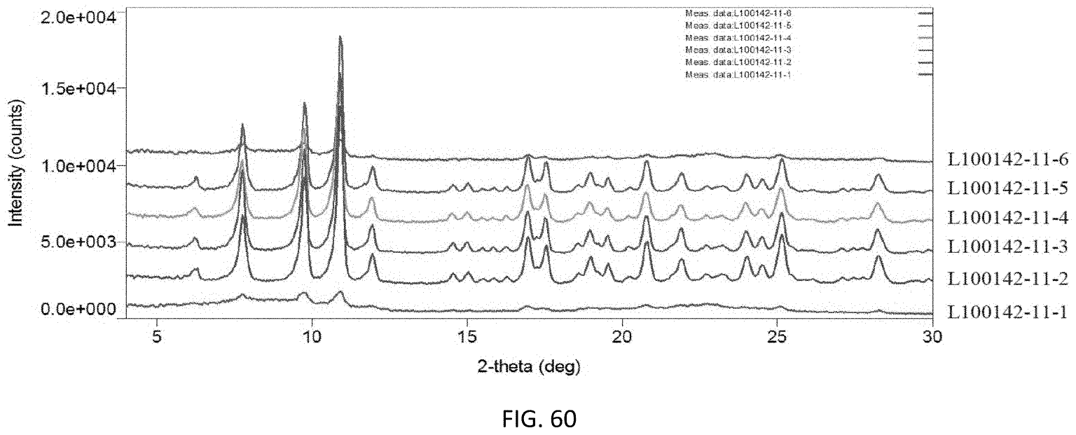

About 25 mg of SJB-01 (L100142-1-1) was weighed into a milling capsule and 25 μL of a chosen solvent (if any) was added. The solid was milled three times, for 30 seconds each time. The solid was scraped off the capsule wall between each 30 seconds of milling to prevent caking. XRPD of the milled material showed no change in form. The dry milled material and that milled with MtBE were of substantially lower crystallinity than the starting material. Table 16 summarizes the experiment conditions and results and XRPD are illustrated in FIG. 60.

TABLE 16
Result of solvent drop milling. XRPD are illustrated in FIG. 60.

| L100142- | Solvent | XRPD pattern |
|---|---|---|
| 11-1 | N/A | Low crystalline β |
| 11-2 | MeOH:water(85:15) | β |
| 11-3 | EtOH | β |
| 11-4 | IPA | β |
| 11-5 | MIBK | β |
| 11-6 | MtBE | Low crystalline β |

Salt Disproportionation

In this set of experiments, strong acid were used to form in-situ salt in the liquid and then disproportionate it into freebase by adding water. It was already observed that the salt does not precipitate as a solid. Disproportionation from various acidic solutions could potentially result in different polymorphs of freebase.

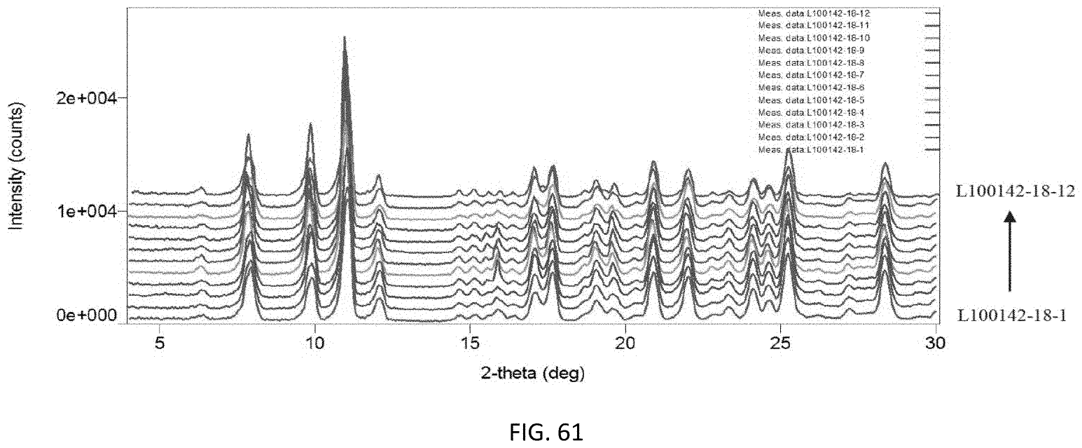

Salts were formed by dissolving about 22 mg of SJB-01 freebase (L100142-1-1) in 2.2 vol. of EtOH and adding the CI. HCl, HBr, H2SO4 and methanesulfonic acid were delivered as 20% vol. solutions in EtOH, Benzenesulfonic acid was delivered as a 37 wt.% solution in EtOH. The solutions added roughly another volume of EtOH to the total volume. Toluenesulfonic acid was added as a solid. The reaction mixtures were stirred at the appropriate temperature, all solutions remained clear and free of precipitate. Fifteen volumes of water were added to the salt solutions in one shot and stirring continued. White slurries rapidly developed at room temperature (22-23° C.) (<5 min), and after about 15 minutes at 50° C., with the development of a slurry being noticeably slower in L100142-18-8 (˜20 mins). The resultant solids were characterized by XRPD, all exhibited Pattern β, illustrated in FIG. 61. No new forms were observed. These experiments also showed that it is unlikely that any salts of SJB-01 would form. Data are summarized in Table 17.

TABLE 17
Summary of data for in-situ salt formation in EtOH
and disproportionation with water. Corresponding
XRPD patterns are illustrated in FIG. 61.

| L100142- | CI | Solvent | Temperature | XPRD |
|---|---|---|---|---|
| 18-1 | Benzenesulfonic acid | EtOH | RT | β |
| 18-2 | Hydrobromic acid | EtOH | RT | β |
| 18-3 | Hydrochloric acid | EtOH | RT | β |
| 18-4 | Methanesulfonic acid | EtOH | RT | β |
| 18-5 | Sulfuric acid | EtOH | RT | β |
| 18-6 | Toluenesulfonic acid | EtOH | RT | β |
| 18-7 | Benzenesulfonic acid | EtOH | 50° C. | β |
| 18-8 | Hydrobromic acid | EtOH | 50° C. | β |
| 18-9 | Hydrochloric acid | EtOH | 50° C. | β |
| 18-10 | Methanesulfonic acid | EtOH | 50° C. | β |
| 18-11 | Sulfuric acid | EtOH | 50° C. | β |
| 18-12 | Toluenesulfonic acid | EtOH | 50° C. | β |

Amorphous Generation

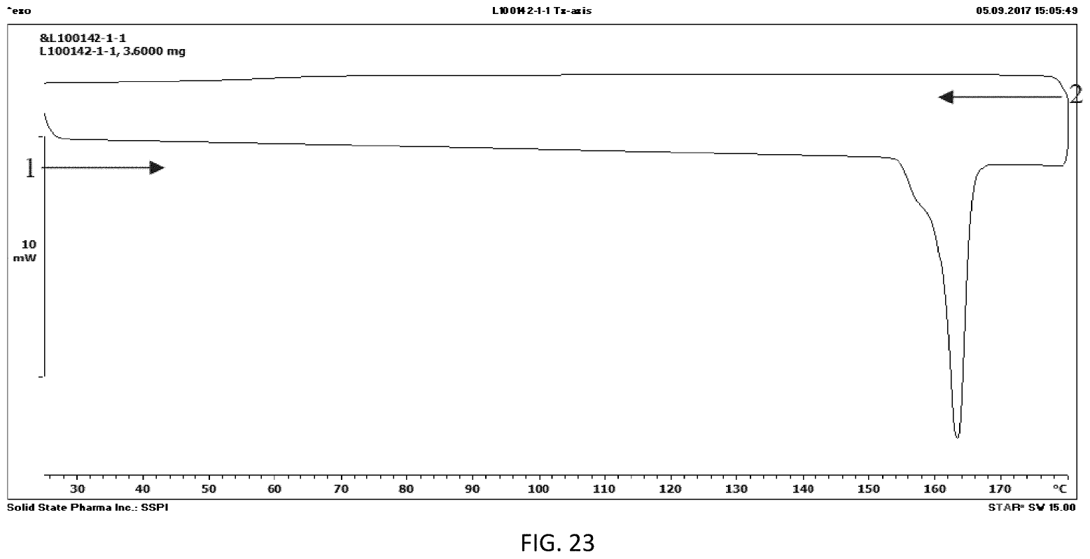

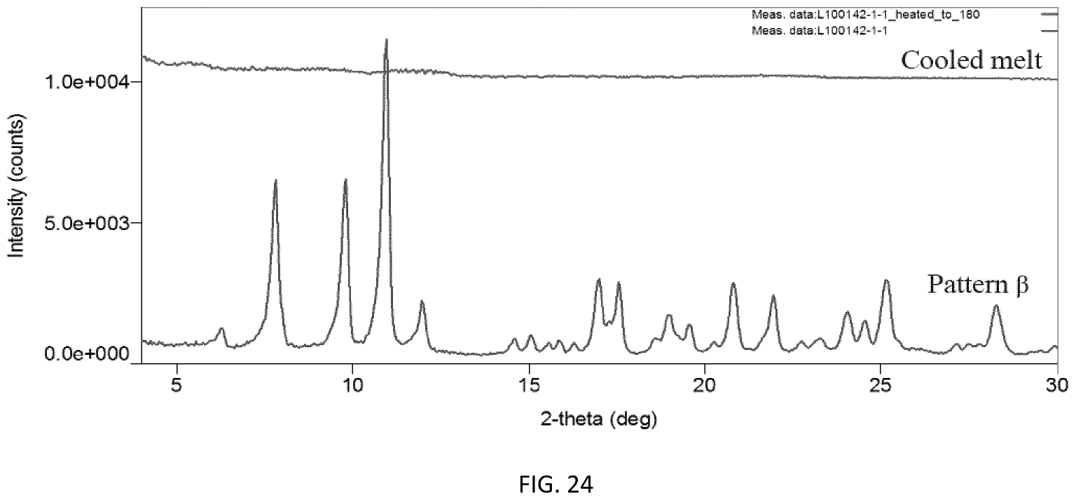

Freebase Pattern β (L100142-1-1) was heated to 180° C. to completely melt the sample, then cooled to 25° C. and observed by DSC (FIG. 23). No exotherm was observed upon decreasing the temperature, a clear colorless glass (brittle) was recovered from the pan and characterized by XRPD. The glass yielded a characteristically amorphous pattern (see blue trace, FIG. 24). Thus, it is deduced that cooling the liquid freebase melt yields amorphous material.

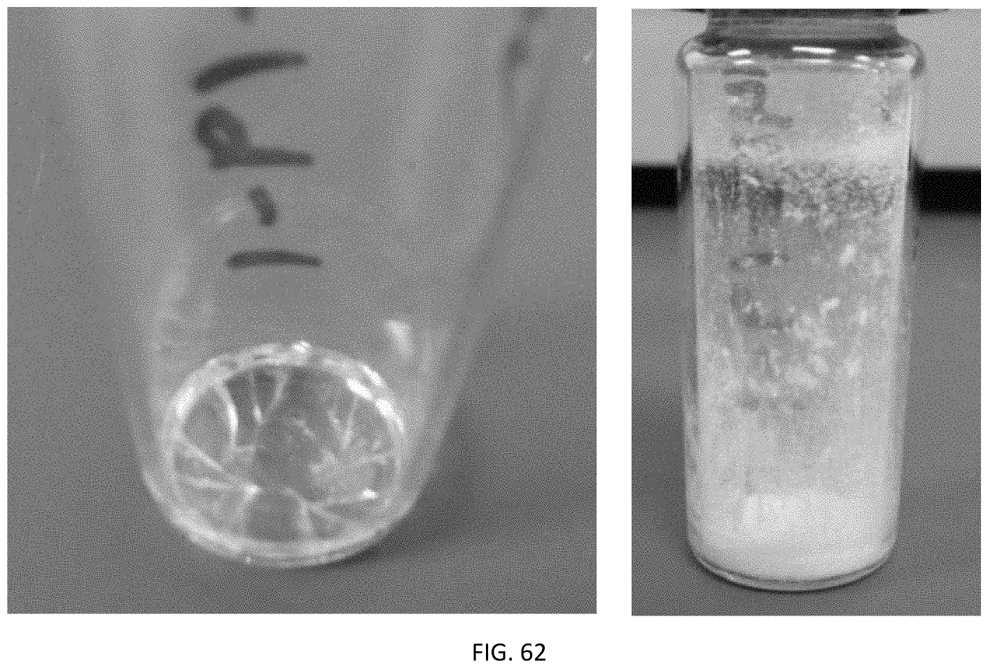

Amorphous freebase was generated by heating SJB-01 Pattern (3 (L100142-1-1) to 175° C. in a heating block to melt, then cooling the melt (heating plate turned off and allowed to cool) to yield a clear, colorless, glassy solid (FIG. 62, left). This brittle solid was scraped and crushed with a needle and spatula to a white, flowable powder (FIG. 62, right).

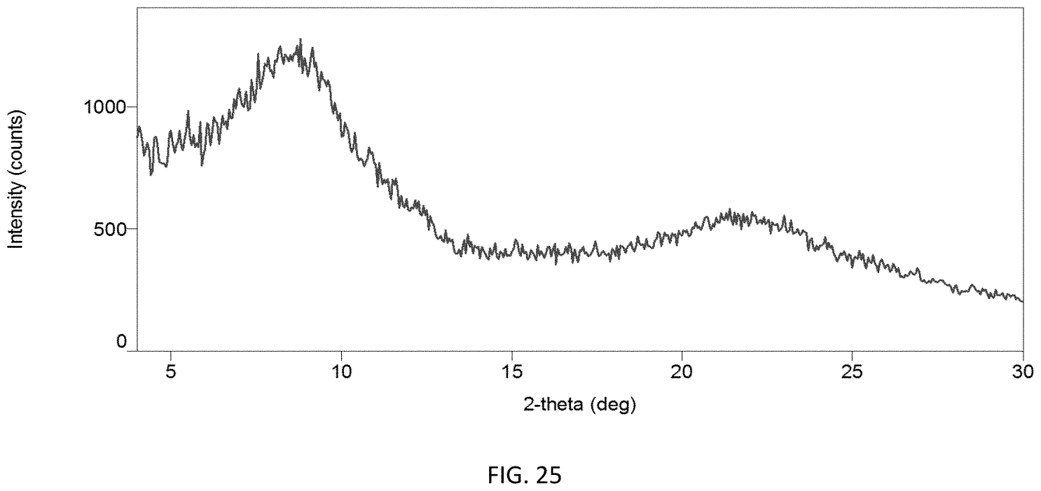

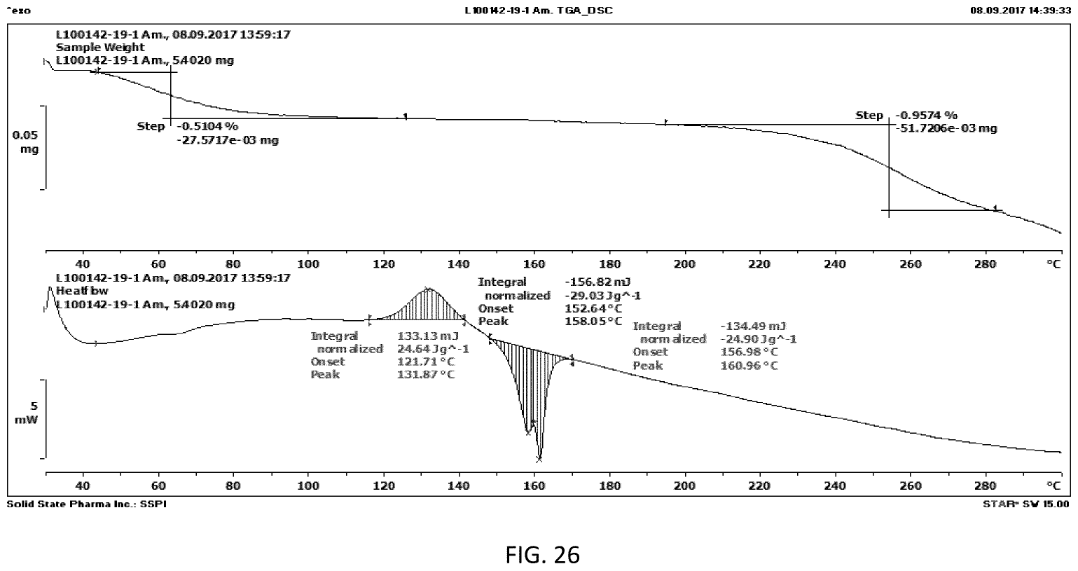

The flowable amorphous powder (L100142-19-1) yielded a characteristically amorphous pattern by XRPD (FIG. 25). Thermal analysis by TGA/DSC (FIG. 26) shows a weight loss of 0.51% between 50 and 125° C. in the TGA thermogram. There are a number of features in the DSC thermogram, including an exotherm with an onset temperature of 121.7° C., indicative of crystallization of the amorphous material, followed by two overlapping endotherms with onsets of 152.6 and 157.0° C. These two overlapping endothermic peaks are the melting events of Patterns α and β, respectively.

Heat Treatment of Amorphous SJB-01 Freebase

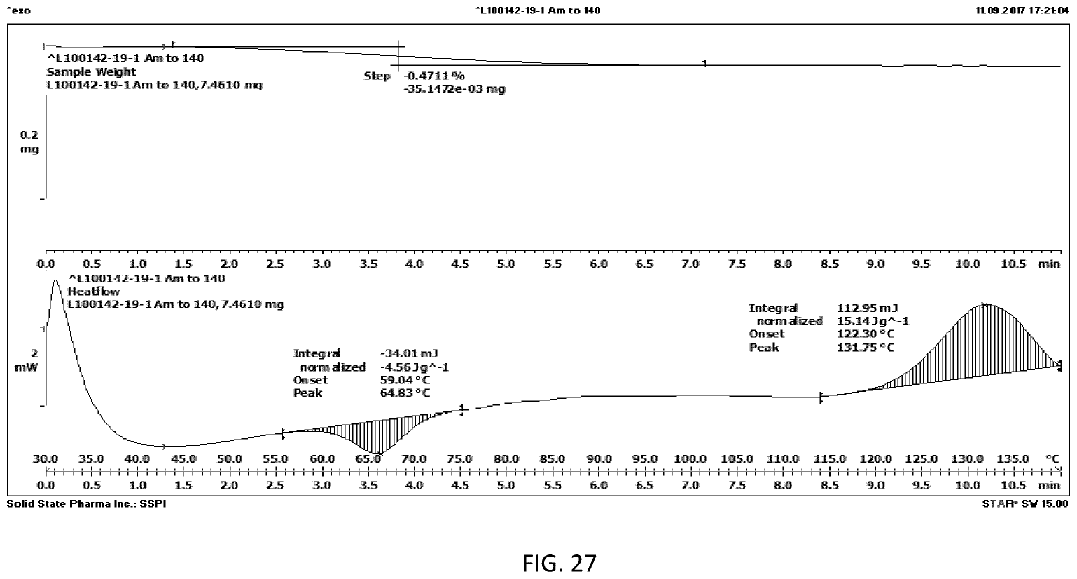

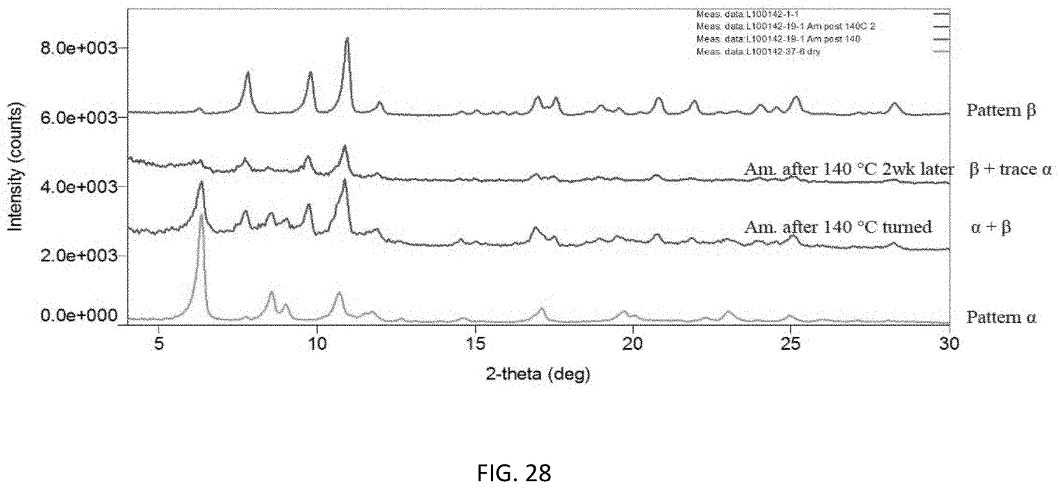

TGA/DSC was performed on a sample of free-flowing amorphous freebase 100142-19-1 up to the temperature of 140° C. (FIG. 27). The expected exotherm with an onset of ˜120° C. was observed. XRPD analysis of the post experiment pan contents showed a mixture of Patterns α and β (FIG. 28). The material was analyzed by XRPD two weeks later and the pattern observed had changed where Pattern α almost completely converted to Pattern β indicating that Pattern α is not stable under ambient conditions.

Slurry of Amorphous Freebase

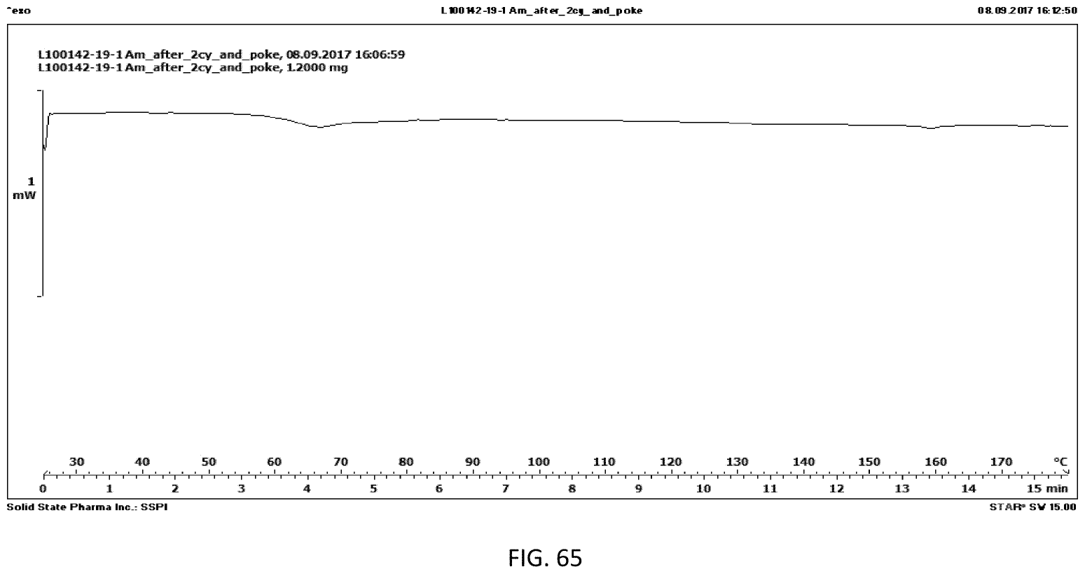

Amorphous freebase was generated by heating ˜15 mg of SJB-01 freebase Pattern β (L100142-1-1) in a vial to 175° C. to melt and allowing the liquid to cool. To the clear colorless glass was added 75 μL (-5 vol.) of one of 6 solvents and the contents stirred. White slurries developed readily (<10 min) in IPA, IPA:water (9:1 vol.), IPAc, and MtBE. The collection and analysis of these solids by XRPD show that all are Pattern β (FIG. 65). After 36 hrs, solids from water and cyclohexane were collected for analysis, also yielding Pattern β (FIG. 65). A summary of data is presented in Table 18.

TABLE 18

| L100142- | Solvent | μL | Comments | XRPD |
|---|---|---|---|---|
| 14-1 | IPA | 75 | Rapid development of while | β |
| | | | slurry. | |
| 14-2 | Water | 75 | Solids collected after 36 hrs. | β |
| 14-3 | IPA:water | 75 | Rapid development of white | β |
| | (9:1 vol.) | | slurry. | |
| 14-4 | IPAc | 75 | Rapid development of white | β |
| | | | slurry. | |
| 14-5 | MtBE | 75 | Rapid development of white | β |
| | | | slurry. | |
| 14-6 | Cyclohexane | 75 | Solids collected after 36 hrs. | β |

Amorphous Stability by XRPD

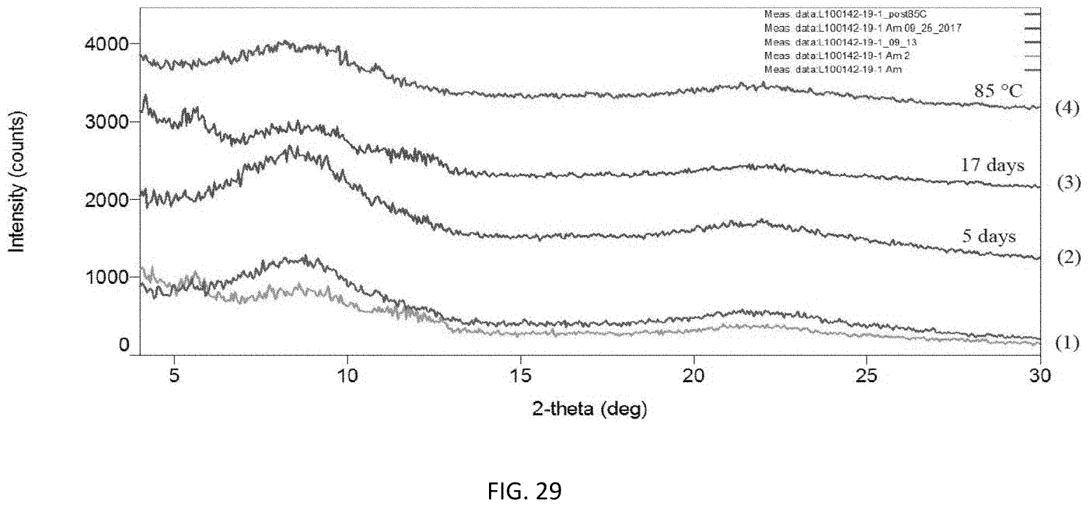

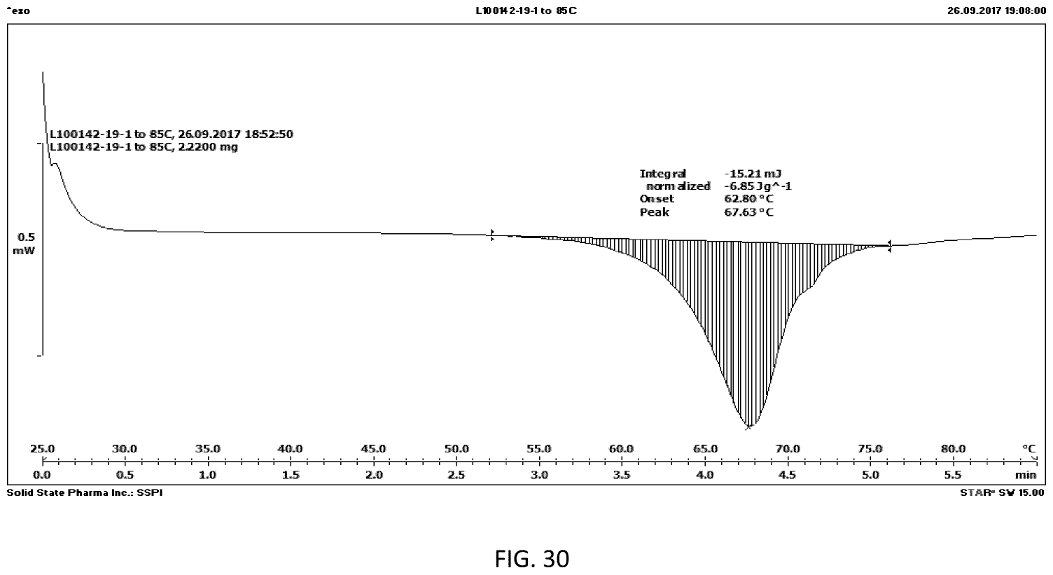

The powdered amorphous freebase was characterized by XRPD after 5 and 17 days and still showed an amorphous pattern (FIG. 29). Heating beyond the small endotherm that peaks at ˜62° C. (the sample was heated to 85° C., see FIG. 30) also made no difference in the observed pattern of the solid.

Stability in High Humidity

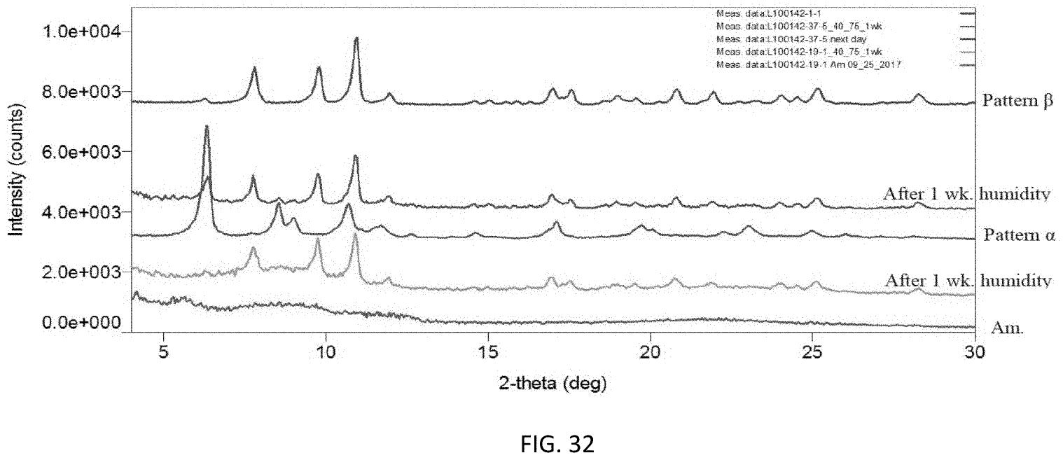

Patterns α (L100142-37-5), β (L100142-1-1) and free flowing amorphous solid (L100142-19-1) SJB-01 were exposed to a 40° C./75% RH environment for one week. XRPD of the solids after the experiment show that α and the amorphous solid had converted considerably to Pattern β. See FIG. 32.

Solubility in Water and Simulated Fluids

The solubility of Patterns α, β and the amorphous freebase was measured in water, FaSSIF (fasted state simulated intestinal fluid) and FaSSGF (fasted state simulated gastric fluid) at 37° C. The solids were slurried in the respective solution for 24 hrs then the liquid isolated by syringe filtration for analysis by HPLC. The data are summarized in Table 19; the solubility of all forms in all solutions is on the microgram scale. Essentially, none of these are substantially advantageous over the other ones in terms of equilibrium solubility.

TABLE 19
Solubility of SJB-01 Pattern α, β, and free-flowing amorphous freebase
in water FaSSIF and FaSSGF at 37° C. after 24 hrs.

| | | | Solubility |
|---|---|---|---|
| L100142- | Fluid | Pattern | (mg/mL) |
| 38-1 | Water | β | 0.005 |
| 38-2 | | Amorphous | 0.005 |
| 38-3 | | α | 0.006 |
| 38-4 | FaSSIF | β | 0.008 |
| 38-5 | | Amorphous | 0.018 |
| 38-6 | | α | 0.008 |
| 38-7 | FaSSGF | β | 0.006 |
| 38-8 | | Amorphous | 0.006 |
| 38-9 | | α | 0.007 |

Characterization and Discussion of Solid Forms

Pattern α

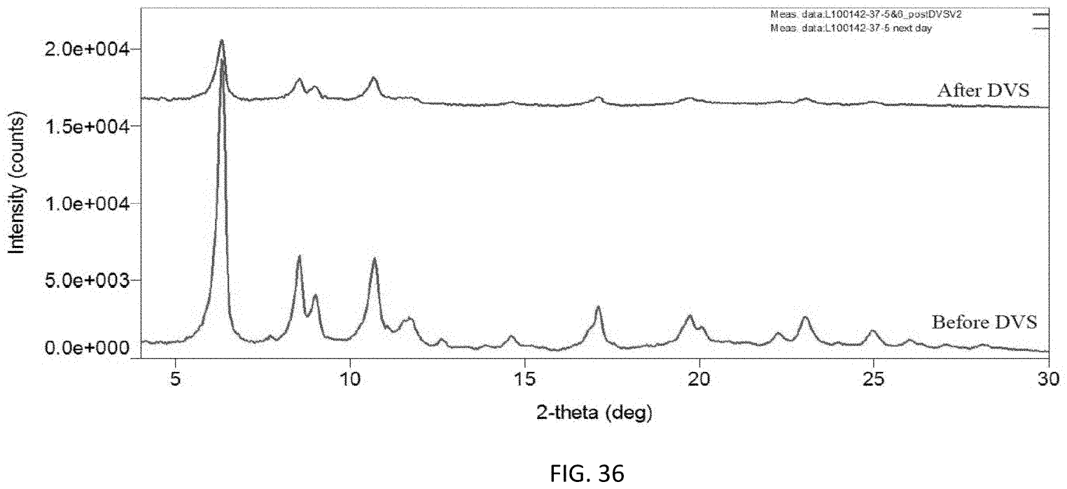

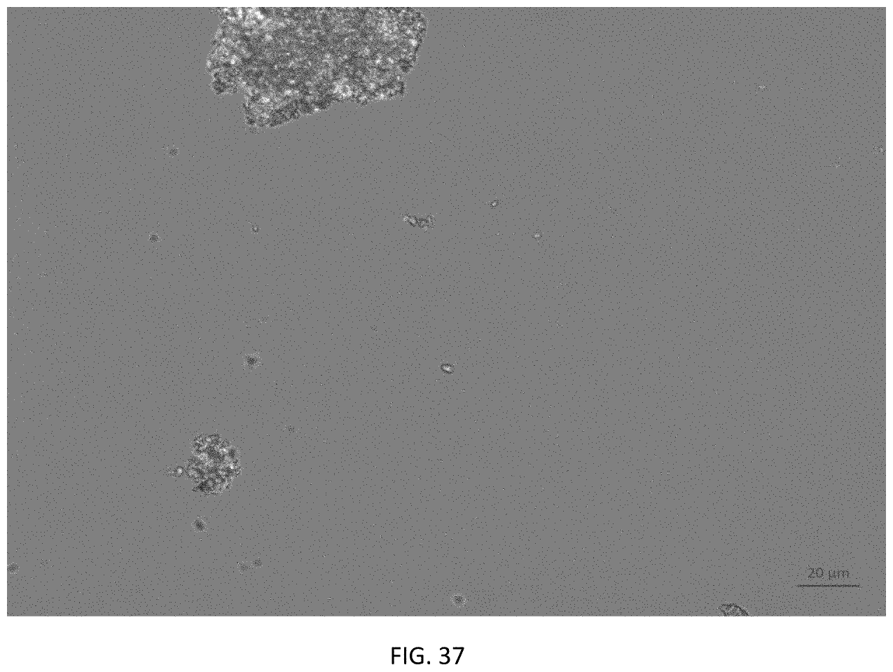

Pattern α is characterized using XRPD, DSC, DVS, and visually using microscope images. The XRPD pattern obtained for Pattern α (sample L100142-37-1a) is provided in FIG. 33 and the various XRPD peaks are identified in Table 20. A DSC thermogram of dried Pattern α (L100142-37-6), showing two melting endotherms characteristic of both Patterns α and β is provided in FIG. 34. There is a possibility that Pattern α recrystallizes as Pattern β upon melting, followed by melting of Pattern β. A DVS isotherm plot for Pattern α (L100142-37-5) is provided in FIG. 35. XRPD patterns for Pattern α (L100142-37-5) before (bottom) and after (top) DVS are shown in FIG. 36. A microscope image of Pattern α (L100142-37-6) is provided in FIG. 37.

TABLE 20
XRPD peak list of Pattern α

| 2θ | d spacing (A°) | Relative intensity, % |
|---|---|---|
| 6.37 | 13.8598 | 100.0 |
| 8.60 | 10.2690 | 16.6 |
| 9.06 | 9.7515 | 6.7 |
| 10.72 | 8.2474 | 13.1 |
| 11.76 | 7.5200 | 4.9 |
| 12.71 | 6.9602 | 2.7 |
| 14.62 | 6.0528 | 1.8 |
| 17.13 | 5.1719 | 8.3 |
| 19.74 | 4.4947 | 3.7 |
| 20.14 | 4.4054 | 1.4 |
| 22.28 | 3.9873 | 2.0 |
| 23.02 | 3.8604 | 4.6 |
| 24.99 | 3.5600 | 2.3 |
| 26.03 | 3.4198 | 0.8 |
| 27.07 | 3.2919 | 0.5 |

Pattern β

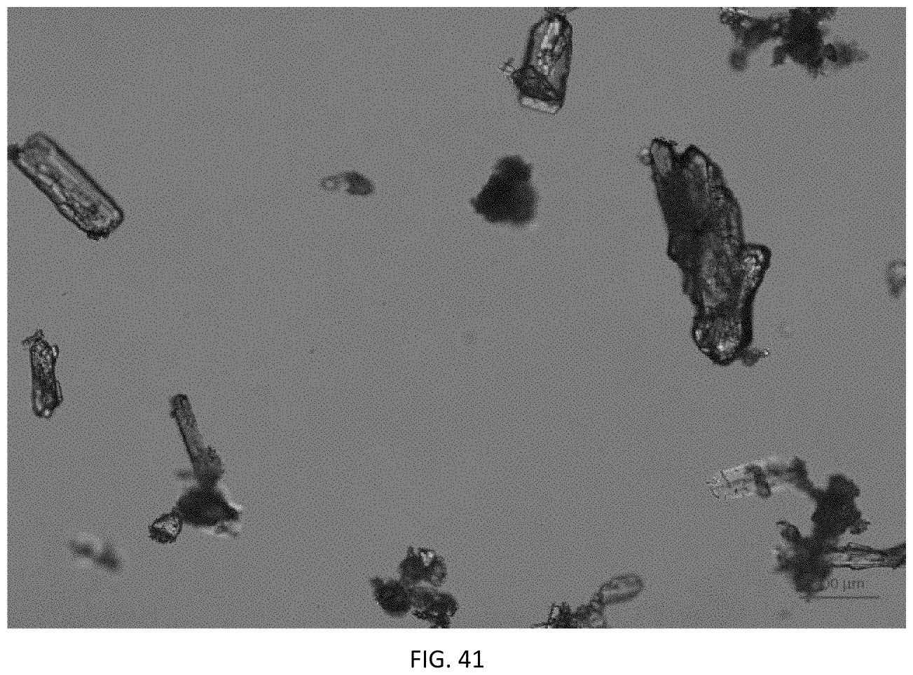

Pattern β is characterized using XRPD, TGA/DSC, DVS, and visually using microscope images. The XRPD pattern obtained for SJB-01 (Lot#2223168, L100142-1-1), Pattern β is provided in FIG. 38 and the various XRPD peaks are identified in Table 21. A TGA/DSC thermogram of SJB-01 (Lot#2223168, L100142-1-1), Pattern β is provided in FIG. 39. A DVS isotherm plot for SJB-01 (Lot#2223168, L100142-1-1), Pattern β is provided in FIG. 40. There is no change in form after the experiment. A microscope image of SJB-01 (Lot#2223168, L100142-1-1), Pattern β is provided in FIG. 41.

TABLE 21
XRPD pattern of Pattern β.

| 2θ | d spacing (A°) | Relative intensity, % |
|---|---|---|
| 6.29 | 14.0457 | 6.0 |
| 7.80 | 11.3235 | 56.6 |
| 9.79 | 9.0295 | 51.8 |
| 10.93 | 8.0861 | 100.0 |
| 11.95 | 7.3975 | 14.7 |
| 14.58 | 6.0716 | 4.4 |
| 15.06 | 5.8763 | 6.0 |
| 15.57 | 5.6879 | 3.2 |
| 15.86 | 5.5828 | 3.8 |
| 16.31 | 5.4308 | 2.9 |
| 16.95 | 5.2255 | 21.1 |
| 17.58 | 5.0415 | 19.3 |
| 18.94 | 4.6825 | 10.9 |
| 19.57 | 4.5335 | 9.0 |
| 20.23 | 4.3864 | 2.6 |
| 20.80 | 4.2672 | 22.0 |
| 21.95 | 4.0458 | 14.5 |
| 22.76 | 3.9037 | 2.7 |
| 23.33 | 3.8098 | 3.0 |
| 24.02 | 3.7014 | 11.7 |
| 24.55 | 3.6230 | 9.3 |
| 25.17 | 3.5348 | 24.7 |
| 27.07 | 3.2916 | 4.5 |
| 27.80 | 3.2064 | 2.1 |
| 28.25 | 3.1565 | 14.6 |

Amorphous

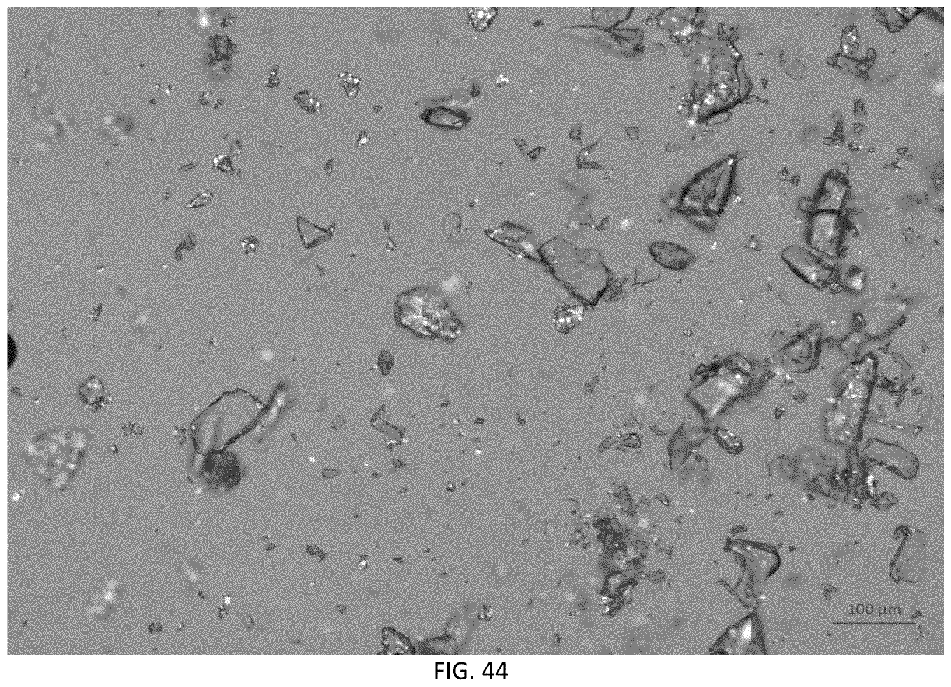

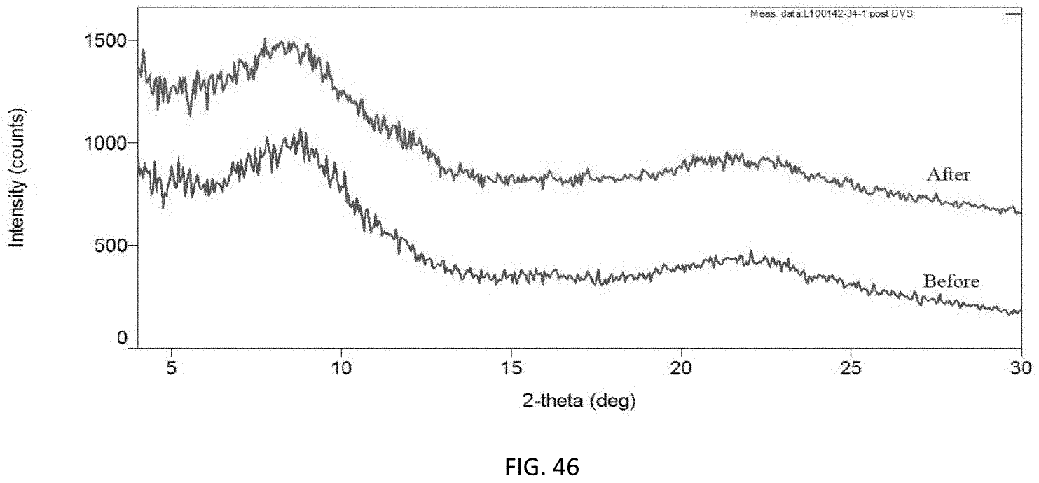

The amorphous solid is characterized using XRPD, TGA/DSC, DVS, and visually using microscope images. The XRPD pattern obtained for the amorphous solid is provided in FIG. 42. A TGA/DSC thermogram of the amorphous solid is provided in FIG. 43. A DVS isotherm plot for powdered amorphous freebase (L100142-34-1) is provided in FIG. 45. An XRPD pattern for the amorphous freebase before and after DVS is provided in FIG. 46. A microscope image of free-flowing amorphous freebase (L100142-19-1) is provided in FIG. 44.

Pattern γ

Pattern γ was only obtained as a mixture with Pattern β. Therefore, the characteristic peaks of Pattern γ were identified by subtracting Pattern β. The XRPD of Pattern γ by subtraction from Pattern β is provided in FIG. 47 and the various XRPD peaks are identified in Table 22.

TABLE 22
Characteristic peaks of Pattern γ.

| 2θ | d spacing (A°) | Relative intensity, % |
|---|---|---|
| 4.31 | 20.4935 | 30.5 |
| 5.62 | 15.7040 | 100 |
| 6.71 | 13.1578 | 25.1 |

Instrumentation

Differential Scanning calorimetry (DSC)

Differential scanning calorimetry was performed using a Mettler Toledo DSC3+. The desired amount of sample is weighed directly in a hermetic aluminum pan with pin-hole. A typical sample mass for is 3-5 mg. A typical temperature range is 30° C. to 300° C. at a heating rate of 10° C. per minute (total time of 27 minutes). Typical parameters for DSC are listed below.

P|arameters

| | |
|---|---|
| Method | Ramp |
| Sample size | 3-5 mg |
| Heating rate | 10.0° C./min |
| Temperature range | 30 to 300° C. |
| Method gas | N₂ at 60.00 mL/min |

Dynamic Vapor Sorption (DVS)

Dynamic Vapor Sorption (DVS) was done using a DVS Intrinsic 1. The sample is loaded into a sample pan and suspended from a microbalance. A typical sample mass for DVS measurement is 25 mg. Nitrogen gas bubbled through distilled water provides the desired relative humidity. A typical measurement comprises the steps:

1. Equilibrate at 50% RH

2. 50% to 2%. (50%, 40%, 30%, 20%, 10% and 2%)-Hold minimum of 5 mins and maximum of 60 minutes at each humidity. The pass criteria is less than 0.002% change.

3. 2% to 95% (2%, 10%, 20%, 30%, 40%, 50%, 60%, 70%, 80%, 90%, 95%)-Hold minimum of 5 mins and maximum of 60 minutes at each humidity. The pass criteria is less than 0.002% change.

4. 95% to 2% (95%, 80%, 70%, 60%, 50%, 40%, 30%, 20%, 10%, 2%)-Hold minimum of 5 mins and maximum of 60 minutes at each humidity. The pass criteria is less than 0.002% change.

5. 2% to 50% (2%, 10%, 20%, 30%, 40%, 50%)-Hold minimum of 5 mins and maximum of 60 minutes at each humidity. The pass criteria is less than 0.002% change.

High Performance Liquid Chromatography (HPLC)

Agilent 1220 Infinity LC: High performance liquid chromatography (HPLC) for chemical purity was conducted using an Agilent 1220 Infinity LC. Flow rate range is 0.2-5.0 mL/min, operating pressure range is 0-600 bar, temperature range is 5° C. above ambient to 60° C., and wavelength range is 190-600 nm.

Chemical Purity Parameters

| Mobile Phase A | 0.05% TFA in water | | |
|---|---|---|---|
| Mobile Phase B | 0.0375% TFA in ACN | | |
| Diluent | ACN:water, 1:1 v/v | | |
| Sample concentration | ~0.5 mg/mL | | |
| Injection Volume | 5 μL | | |
| Monitoring Wavelength | 220 nm | | |
| Column | XBridge C18, 4.6 mm × 150 mm, 3.5μ | | |
| Column Temperature | 40° C. | | |
| Flow rate | 1 mL/ min | | |
| Gradient Method | Time (min) | % A | % B |
| | 0 | 95 | 5 |
| | 3 | 95 | 5 |
| | 10 | 5 | 95 |
| | 15 | 5 | 95 |

Microscopy

Optical microscopy was performed using a Zeiss AxioScope A1 equipped with 2.5×, 10×, 20× and 40× objectives and polarizer. Images are captured through a built-in Axiocam 105 digital camera and processed using ZEN 2 (blue edition) software provided by Zeiss.

Nuclear Magnetic Resonance (NMR)

Proton NMR was done on a Bruker Avance 300 MHz spectrometer. Solids are dissolved in 0.75 mL deuterated solvent in a 4 mL vial and transferred to an NMR tube (Wilmad 5 mm thin wall 8″ 200 MHz, 506-PP-8). A typical measurement is usually 16 scans. Typical parameters for NMR are listed below.

Parameters - Bruker Avance 300

| | |
|---|---|
| Instrument | Bruker Avance 300 MHz spectrometer |
| Temperature | 300 K |
| Probe | 5 mm PABBO BB-1H/DZ-GRD Z104275/0170 |
| Number of scans | 16 |
| Relaxation delay | 1.000 s |
| Pulse width | 14.2500 μs |
| Acquisition time | 2.9999 s |
| Spectrometer frequency | 300.15 Hz |
| Nucleus | 1H |

Thermogravimetric Analysis and Differential Scanning Calorimetry (TGA and DSC)

Thermogravimetric analysis and differential scanning Calorimetry was done using a Mettler Toledo TGA/DSC3+. The desired amount of sample is weighed directly in a hermetic aluminum pan with pin-hole. A typical sample mass for the measurement is 5-10 mg. A typical temperature range is 30° C. to 300° C. at a heating rate of 10° C. per minute (total time of 27 minutes). Protective and purge gasses are nitrogen (20-30 mL/min and 50-100 mL/min). Typical parameters for DSC/TGA are listed below.

Parameters

| | |
|---|---|
| Method | Ramp |
| Sample size | 5-10 mg |
| Heating rate | 10.0° C./min |
| Temperature range | 30 to 300° C. |

X-ray Powder Diffraction (XRPD)

Powder X-ray diffraction was done using a Rigaku MiniFlex 600. Samples were prepared on Si zero-return wafers. A typical scan is from 2θ of 4 to 30 degrees, with step size 0.05 degrees over five minutes with 40 kV and 15 mA. A high-resolution scan is from 2θ of 4 to 40 degrees, with step size 0.05 degrees over thirty minutes with 40 kV and 15 mA. Typical parameters for XRPD are listed below.

Parameters for Reflection Mode

| X-ray wavelength | Cu Kα1, 1.540598 Å, |
|---|---|
| X-ray tube setting | 40 kV, 15 mA |
| Slit condition | Variable + Fixed Slit System |
| Scan mode | Continuous |
| Scan range (°2TH) | 4-30 |
| Step size (°2TH) | 0.05 |
| Scan speed (°/min) | 5 |

Conclusions

Baseline characterization was performed on the base solid compound. These characterizations include XRPD, DSC/TGA, DVS, proton NMR and polarized light microscopy (PLM). The solid (Lot#2223168) was designated as Pattern β. XRPD showed that the solid is crystalline. TGA showed about 0.1% weight loss from 30° C. to 180° C. for Pattern β. One thermal event was observed in the DSC thermogram with the peak at 162.9° C. in the temperature range of 30° C. to 300° C. Dynamic vapor sorption of Pattern β showed that the solid picks up very little moisture, about 0.1% from 2% to 95% relative humidity. The XRPD remained unchanged after DVS. PLM revealed the solid has a large rod-like morphology with length of some particles over 100 μm. Humidity test was conducted on the solid with exposure to 75% relative humidity at 40° C. for 1 week followed by XRPD analysis. The XRPD remained unchanged after one week test. Quantitative solubility measurement was performed through gravimetric method in 14 different solvents at two temperatures (23° C. and 50° C.). Solubility was moderate to high in most of the organic solvents.

Polymorph screening was conducted using a wide range of solvents and techniques: slurry experiments, cooling crystallization (fast, slow and stagnant to −20° C.), anti-solvent crystallization (direct and reverse), evaporative crystallization (flash, rotavap, stirred, stagnant), solvent drop grinding, disproportionation of in-situ salt, crystallization from gel and amorphous slurry in solvents. About 240 samples were generated and analyzed by XRPD. The result from screening was three Patterns: α, β and γ, the latter of which was mixed with Pattern β.

Pattern β was most frequently observed pattern. Pure Pattern α was produced through evaporation of a 1-propanol solution. Pattern γ was produced once through saturating a solution of API in ethyl acetate at room temperature followed by storing at −20° C. for one week. This resulted in Pattern γ mixed with Pattern β. This pattern however, was not reproduced again.

Pattern α was found to be crystalline and less stable than Pattern β. The melting peak of Pattern α was 157.1° C. Dynamic vapor sorption of Pattern α showed that the solid picks up very little moisture, about 0.6% from 2% to 95% relative humidity. The XRPD remained unchanged after DVS. The solubility of Pattern α in water and simulated fluids was in the range of 6-8 μg/mL.

Amorphous solid was generated by melting the API at 175° C. followed by rapid cooling to room temperature which resulted in a hard gel-like material. Upon crushing the hard gel, a flowable powder was obtained which was found to be amorphous by XRPD. The solubility of amorphous solid in water and simulated fluids was in the range of 5-18 μg/mL. The amorphous form remained stable at ambient environment for at least 17 days.

### Example 3

### Additional Data Characterizing SJB-01

The chemical name of SJB-01 is 2-Ethylamino-8-fluoro-3-methyl-1-oxo-1,2-dihydro-isoquinoline-4-carboxylic acid ((S)-cyclopropyl-phenyl-methyl)-amide and it has a chiral chemical structure as shown here:

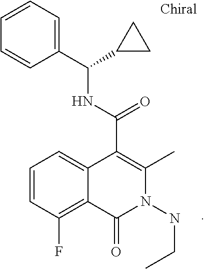 [SMILES: CCNn1c(C)c(C(=O)N[C@H](c2ccccc2)C2CC2)c2cccc(F)c2c1=O]

SJB-01 has a conversion factor of 1 mg˜2.54 μmol; 1 mol/L˜393.46 mg/mL.

The pKa value was measured by titration. SJB-01 is a weak base with a pKa value below the measurable detection limit of 2.

The Log P/D_{7.4} value was determined by the shake flask method in an octanol-buffer two phase systems. Log P/D_{7.4} is measured to 3.3, which indicates that the compound has lipophilicity suitable for penetration through the blood brain barrier.

Multiple batches of SJB-01 were prepared and the CHN, DSC, TGA, KF, and X-ray results of the various prepared free bases were summarized in Table 27.

TABLE 27
CHN, DSC, TGA, KF results and polymorph form of the prepared SJB-01 bases.
CHN values in bold correspond to the theoretical content in the free base.

| | | | | | DSC | | TGA | |
|---|---|---|---|---|---|---|---|---|
| | | | | | (Tₒₙₛ/ | | % weight | Polymorph |
| | | | | % | Tₚₑₐₖ) | ΔHₘ | loss | Form |
| Batch | % C | % N | % H | Water | ° C. | (J/g) | (−>Temp.) | (x-ray) |
| Base: | 70.21 | 10.68 | 6.15 | | | | | |
| 60119-80 | 70.26 | 10.66 | 6.11 | | 153.9/157.2 | 52 | <0.1 (165) | Alpha |
| 60095-057 | 70.05 | 10.57 | 6.17 | | | | | Alpha |
| 60215-26E | 70.35 | 10.70 | 6.16 | <0.1 | 152.3/157.7 | 42 | <0.1 (150) | Alpha |
| 60317-001E | 70.57 | 7.97 | 5.88 | 0.25 | 154.9/158 | 43 | 0.6 (160) | |
| 60095-076 | 70.12 | 10.58 | 6.19 | | | | | Beta |
| 60095-056 | | | | | 153.7/156.5 | 48 | | Beta |
| 60119-83 | 70.14 | 10.62 | 6.18 | | | | | Alpha |

SJB-01 was found to be stable when exposed to oxygen and a pH range of 2, 4, 6, and 7.4. However, in aqueous solution and under extreme light exposure, 100% of the compound was decomposed after 24 hr. Solid compound was found to be slightly degraded under this extreme light exposure after 24 hr where ˜86% of the compound is left. In the non-aqueous vehicle, polyethylene glycol (PEG) 400, the compound was found to be stable for at least 4 months when kept at room temperature and covered from light.

Among the three crystal forms and amorphous solid, Pattern β was the most stable polymorph. None of the solid forms showed superior solubility in water and simulated fluids.

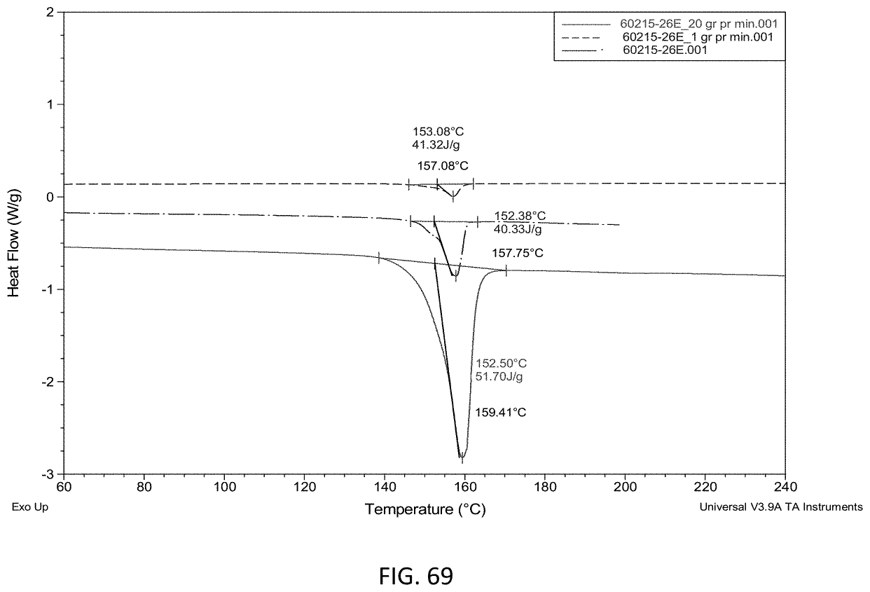

DSC measurements at three different heating rates were done on an alpha and a beta batch (2009-V-0680). For batch 60215-26E (alpha) the onset temperature is between 152.3 and 152.9° C. when varying the heating rate (FIG. 69).

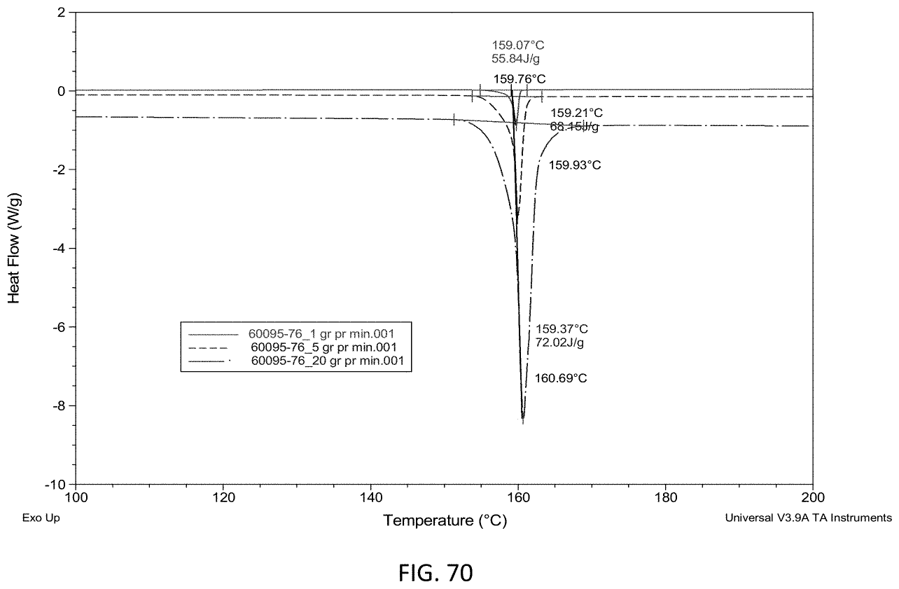

For batch 60095-76 (beta) the onset temperature lies between 159.1 and 159.4° C. (FIG. 70). The narrow interval suggests pure melting. The higher melting point of beta combined with the slightly higher melting enthalpy (see values in FIG. 69 and FIG. 70) suggests that beta is the stable form.

The melting temperature of beta batch 60095-76 is somewhat higher than seen for batch 60095-056/6 (153.7° C.-also beta), which might reflect that the latter contains impurities (also reflected in a broad melting interval). Also, for some alpha batches, melting takes place over a broader interval.

Two batches of both alpha and beta polymorphic forms have been heated on a hot stage microscope, and pictures have been recorded at different temperatures. The samples were heated at a heating rate of 20° C/min until 155° C. Heating was then continued in steps of 1° C., pausing for approximately 2 min at each temperature (2009-V-678).

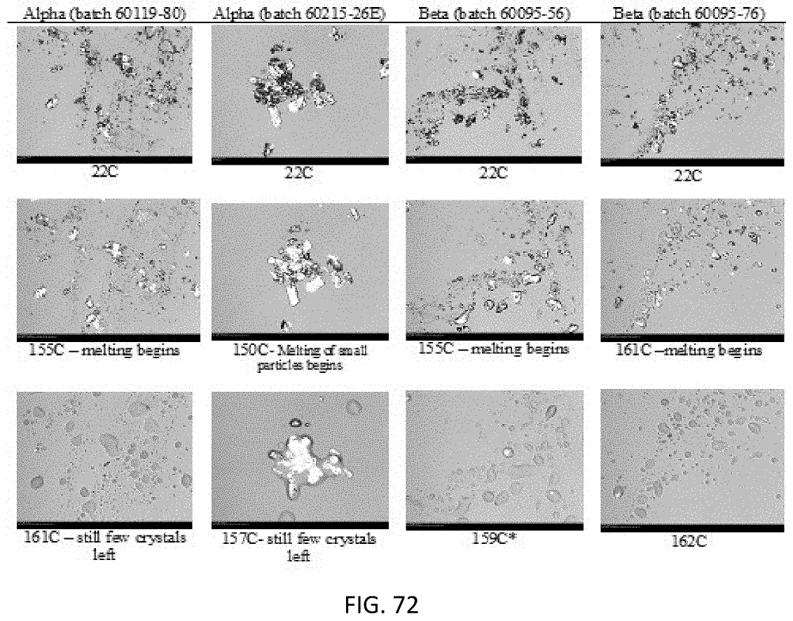

The crystals formed at room temperature were similar for one alpha batch and two of the beta batches: they formed small cubes of varying size (FIG. 72). For the other alpha batch (60215-26E) larger cubes were observed.

For alpha, batch 60215-26E melting begins at a lower temperature than for the other batches.

When heating beta, batch 60095-56, appearance of needles around the melting point (159° C.) were seen. This might be due to formation of a different polymorph. For two of the batches (60215-26E and 60095-56) melting took place over a quite broad temperature interval which is confirmed by the DSC results. This might be due to impurities or transformation during melting. For beta, batch 60095-76, melting took place over a much narrower interval and this may reflect melting of the pure beta-form.

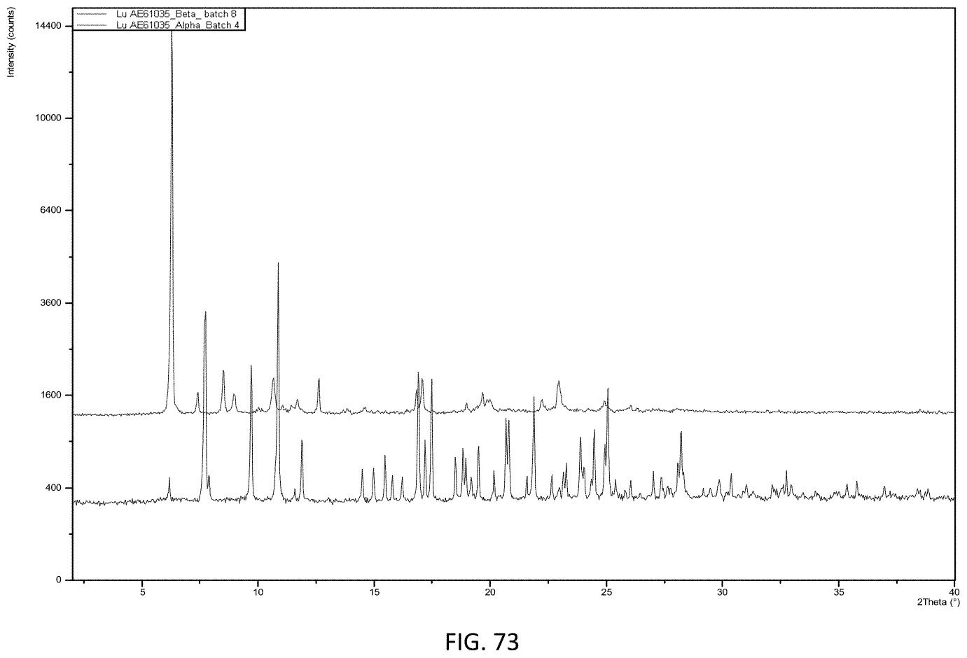

For SJB-01 two polymorphic forms were initially discovered among the synthesized batches and were shown using x-ray powder diffraction (XRPD) (FIG. 73). Batches 4 and 7 were designated the α-form, whereas batch 6 and 8 were a β-form. Melting point for both forms was 152-154° C.

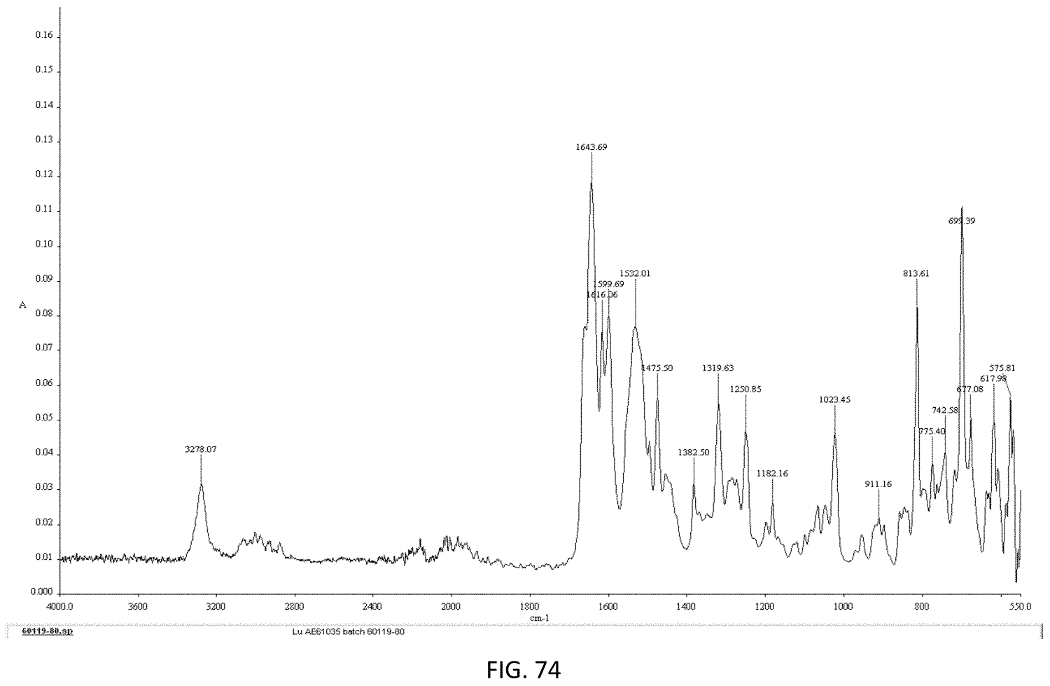

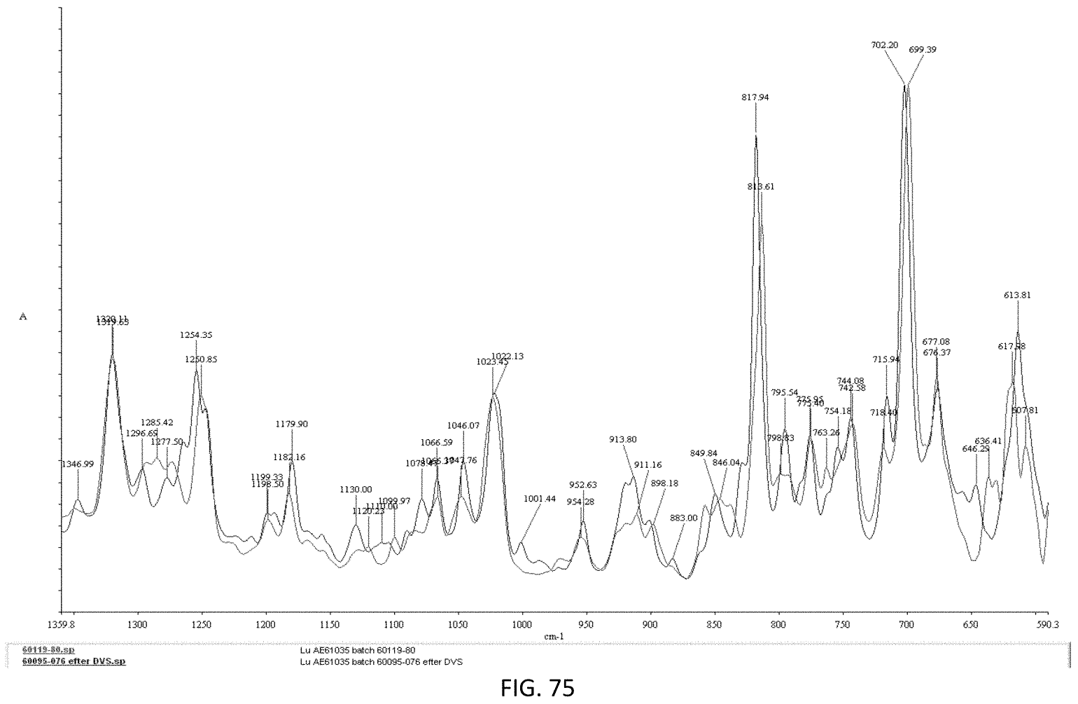

In addition, infrared spectroscopy was obtained for SJB-01. Only small differences were observed between the two polymorphic forms of SJB-01, summarized in Table 23 and seen in FIGS. 74-75. The slight differences indicated that the binding pattern in the two crystal structures was almost identical.

TABLE 23
IR peak positions (cm⁻¹) differing between the alpha and beta-form

| Alpha | Beta |
|---|---|
| 1250.8 | 1254.3 |
| | 1078.5 |
| | 1001.4 |
| 813.6 | 817.9 |
| 636.4 | 646.3 |

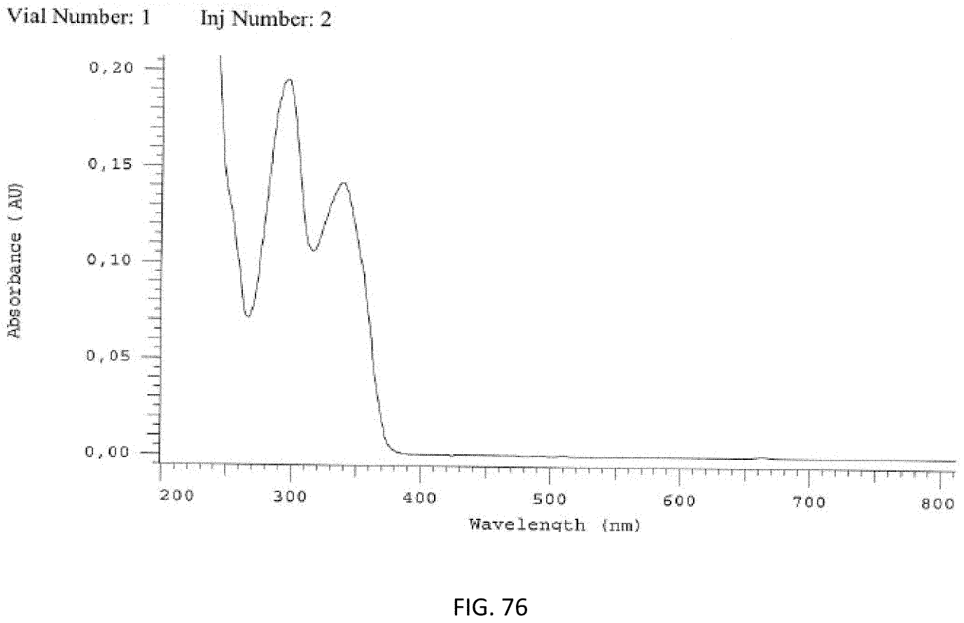

The UV-vis spectrum of the compound displayed absorbance in the region 290-700 nm (FIG. 76).

The aqueous solubility of SJB-01 is low and the thermodynamic equilibrium solubility is measured to be ˜8 μg base/ml at pH 7.4 for the β-polymorph form, and about 14 μg/ml for the a-polymorph form.

In addition, the solubility of SJB-01 was investigated in a large number of vehicles for use in in vivo pharmacological studies, as well as toxicological evaluation of the compound. A selection of the vehicles tested are shown in Table 24.

TABLE 24
Solubility of SJB-01 free base in various excipients

| Formulation | mg base/ml * |
|---|---|
| 10% HPbetaCD | 0.03 |
| 25% Solutol HS15 | 0.7 |
| 25% Cremophor EL | 0.9 |
| 100% Softigen 767 | 10.6 |
| 100% PEG 400 | 22 |
| Gelucire 44/14:PEG 400 90:10 | 26 |
| 100% Viscoleo^{§} | 3.8 |

* The saturated solubility has been determined on batches which contain the β-form.
^{§}Medium chain triglyceride

The solubility in hydroxypropyl beta cyclodextrin (HPbetaCD) and other aqueous excipients was very low and subsequently a non-aqueous vehicle has been used for in vivo studies. For experiments performed with subcutaneous administration, an emulsion containing 20% viscoleo stabilized with 1.2% lecithin has been used. For peroral dosing, 100% PEG 400 was used.

Formulations for toxicological evaluation of SJB-01 were considered. The highest solubility was obtained with 100% PEG 400 and a mixture of Gelucire and PEG 400. Since a formulation with Gelucire:PEG required increased handling time, including dosing at ˜45° C., 100% PEG 400 was chosen for formulation purpose in the early toxicological evaluation including mini-MTD (maximum tolerated dose) and a 14 days toxicological dosing study. The maximum dose reached was 140 mg/kg-the highest possible dose achievable with PEG 400 as vehicle. Since no clinical observations were obtained at this dose, it was considered whether the dose could be further increased. From a formulation perspective, this can only be done by using Gelucire:PEG as a vehicle, under the assumption that a solution will lead to a higher bioavailability than dosing the compound in a PEG 400 suspension. In the table below, the maximum dose of SJB-01 given on a solubilized form is provided, taking into account the tolerated dose of the vehicle as well. Furthermore, different polymorph forms of SJB-01 display very different solubilities, as can be seen in Table 25.

TABLE 25
Formulation possibilities in rats during regulatory safety studies

| | 100% PEG 400 | | Gelucire:PEG 400 90:10 | |
|---|---|---|---|---|
| Formulation | mg base/ml | mg/kg^{§} | mg base/ml | mg/kg^{$} |
| α-form | 32 | 140 | 40 | 360 |
| β-form | 22 | 100 | 26 | 230 |

^{§}5 ml/kg is maximum volume dose
^{$}10 ml/kg is maximum volume dose

For regulatory safety studies in dogs, 100% PEG 400 can be used. However, the maximum volume that can be used in 1-2 ml/kg, indicating that maximum single dose in dogs will be 28-56 mg/kg or 20-40 mg/kg for the α- and β-form, respectively, if the compound is given solubilized.

The compound for the toxicological evaluation should be delivered as the free base if maximum concentrations are to be used, as salts significantly decrease solubility in non-aqueous excipients like PEG 400.

The solubility of the beta polymorph form of SJB-01 was further examined in bio-relevant media. The fasted-state small intestinal fluid (FaSSIF) contained 3 mM sodium taurocholate (bile acid), 0.75 mM lecithin (phosphorlipid) and phosphate buffer to maintain a pH of 6.0. The fed-state small intestinal fluid (FeSSIF) contained 5-fold higher concentration of sodium taurocholate and lecithin and thus higher capacity for micellar solubilization of the compound. The pH in the FeSSIF buffer was maintained to 5.0 with acetate buffer. The results are summarized in Table 26.

TABLE 26
Solubility in bio-relevant media of the β-form

| Bio-relevant media | μg base/ml |
|---|---|
| 0.001M HCl pH 3.0 | 8 |
| FaSSIF | 11 |
| FeSSIF | 19 |

The presence of bile components highly increased the solubility of the beta polymorphic form of SJB-01. The enhanced solubilization in the small intestine by bile components will be reflected in humans and hence bioavailability may be higher than anticipated from the low aqueous solubility. Furthermore, if the drug is administered with food, increased solubilization, and hence increased bioavailability, may be expected since solubility is further increased in the FeSSIF media.

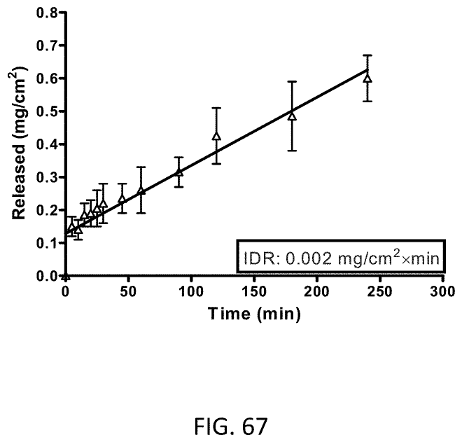

The Intrinsic Dissolution Rate (IDR) was calculated for a beta polymorph form of SJB-01. A tablet of the compound was pressed and put into a dissolution chamber. It was subsequently measured how fast the substance was dissolved from the surface into the solvent. Initially, the experiment was performed in a standard buffer (0.001 M HCl pH 3.0), reflecting the stomach conditions at fastened state. Examination of the IDR indicated that the compound dissolved very slowly. The results showed an IDR of 0.002 mg/cm²·min (FIG. 67).

Due to the high impact of bile components on solubilisation of SJB-01, IDR was performed in FeSSIF media, reflecting the fed-state in humans. Surprisingly, initial experiments showed that the IDR was not further increased in the presence of bile components.

The possibility of dosing SJB-01 orally as a solid was explored in rats, as a means to evaluate the developability of the compound. Clinical relevant doses (1, 5 and 10 mg/kg) were administered to rats as microsuspensions. Furthermore, to explore the role of particle size, a suspension (5 mg/kg) was administered as well for comparison.

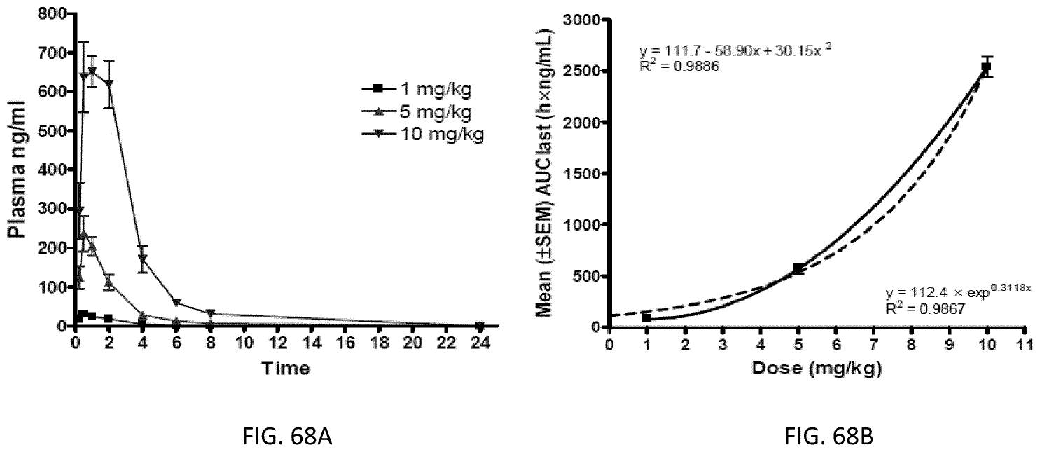

When dosing microsuspensions of SJB-01, the absolute bioavailability was ˜20% at low doses (1 and 5 mg/kg), but increased significantly at 10 mg/kg (˜30%), suggesting that dose proportionality follows an exponential pattern (FIGS. 68A-68B). When comparing particle size (10 and 50 μm) at 5 mg/kg, there were no differences in bioavailability, suggesting that the absorption is not dissolution limited at low doses. The exponential increase in bioavailability at higher doses may indicate that metabolic enzymes or transport systems need to be saturated in order to receive higher bioavailability.

A developability assessment was conducted with regards to SJB-01. The very low aqueous solubility and low pKa value of SJB-01 may lead to limited solubilization in the gastro intestinal tract. To further explore this potential issue, IDR of the compound was measured, and demonstrated a very slow dissolution rate of the compound. Based on these results, it was therefore surprising to see how well solid SJB-01 was absorbed in the rats upon oral administration. When dosing 10 mg/kg SJB-01, as a microsuspension to rats, the absolute bioavailability was quite good (˜30%). This clearly indicates that particle size and stomach and/or intestinal fluid will positively influence the bioavailability of the compound opening for a possible conventional tablet or capsule formulation.

To explain the surprisingly high bioavailability when considering the low IDR, solubility in bio-relevant media was performed. Also, IDR in a fed-state media reflecting the intestinal environment of the rat was measured. The solubility was increased when comparing to the fasted stomach conditions to fasted- and fed-state simulated intestinal fluid. Surprisingly however, IDR was not further improved in a fed-state simulated intestinal fluid as compared to standard IDR media.

The maximal absorbable dose (MAD) of SJB-01, as described by Curatolo (1998), can be calculated according to the following equation:

MAD=S*Kₐ*SIWV*SITT

Where S is the solubility (mg/ml) at pH 6.5, Kₐ is the transintestinal absorption rate constant (0.03 min-1 for well absorbed compounds), SIWV is the small intestinal water volume (here set to 250 ml) and SITT is the small intestinal transit time (assumed to be 4.5 hours=270 min). Using this equation, the maximal absorbable dose is 16 mg for SJB-01. As indicated in Table 28, the MAD calculation can be further elaborated by using solubility from the bio-relevant media. Hence, the MAD is increased up to 38 mg for SJB-01 in a fedstate situation.

TABLE 28
Maximal absorbable dose of SJB-01 in humans

| | MAD - SJB-01 |
|---|---|
| Bio-relevant media | mg* |
| 0.001M HCl pH 3.0 | 16 |
| FaSSIF | 22 |
| FeSSIF | 38 |

*Calculations are based on data from the β-form

The human dose for SJB-01 is predicted to be 5 mg or less (Bundgaard and Steiniger-Brach, Report 046-845-2009). Since the MAD calculation is within the range of predicted human dose, no bioavailability problems with the compound are foreseen.

### Example 4

### Single Crystal Structure of SJB-01, Beta Form Polymorph

An X-ray quality single crystal of SJB-01, Pattern β, was grown by evaporation from a solution of methyl isobutyl ketone and methyl tert-butyl ether (sample L100149-74-13). The data were collected on the crystal (0.22×0.41×0.46 mm³) at 100 K and solved in the monoclinic P2₁ space group. The final residual values of the refinements are R1=0.0229 (I>2σ(I)) and wR2=0.0613 (all data). Solutions with wR2 approximately double the R1 value.

Two molecules of SJB-01 and no solvent comprise the asymmetric unit. Both molecules in the asymmetric unit are S enantiomers (chiral carbon C1 and C31, FIG. 78), the lack of inversion center, mirror, or glide plane symmetry elements in the P2₁ space group necessitates only one enantiomer in the crystal. The assignment of the absolute configuration was done with high confidence based on the refined Flack parameter of 0.016(16). A Flack parameter of 1 would indicate the correct solution is the inverse of the configuration determined, 0.5 would indicate the crystal contains both enantiomers, and 0.0 shows the solution determined is correct.

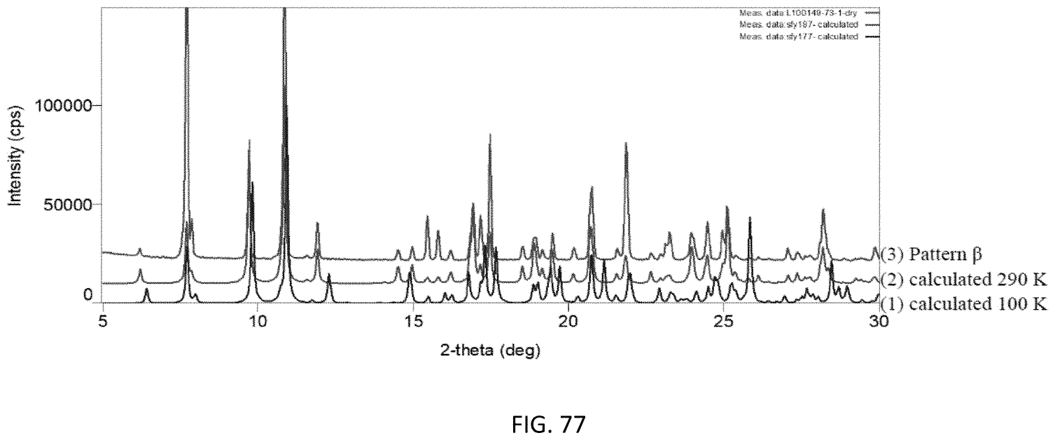

The crystal structure does not show any pores or channels that would suggest issues with solvent inclusion or retention. FIG. 77 shows an overlay of the calculated XRPD pattern from the single crystal data collected at 100 K and 290 K, and the experimental XRPD pattern of Pattern β at room temperature (ca. 298 K). The 100 K and 290 K calculated patterns show some significant differences, and this can be attributed to the considerable change in the c-axis of the unit cell over the temperature range (see Table 29 for unit cell parameters at both temperatures). The 290 K calculated and experimental pattern for Pattern β match.

TABLE 29
Unit cell parameters of β SJB-01 at 100 K and 290 K

| Unit cell | sfy177 | sfy187 |
|---|---|---|
| parameter | 100 K | 290 K |
| a (Å) | 12.3337(4) | 12.3668(2) |
| b (Å) | 11.8844(4) | 11.8421(2) |
| c (Å) | 14.8280(5) | 15.3530(3) |
| α (°) | 90 | 90 |
| β (°) | 111.8983(11) | 112.0189(8) |
| γ (°) | 90 | 90 |
| Volume (Å³) | 2016.65(12) | 2084.43(6) |

Sample Preparation

A sample of compound SJB-01 in a methyl-isobutylketone (MIKB) methyl-tbutylether (MtBE) mixture was obtained. It contained several crystals of good quality. The specimen chosen for data collection was a block with the dimensions of 0.22×0.41×0.46 mm³. The crystal was mounted on a MiTeGen™ mount with mineral oil (STP Oil Treatment). First diffraction patterns showed the crystal to be of good quality without signs of non-merohedrally twinning.

Data Collection and Data Reduction

Diffraction data (φ- and ω-scans) were collected at 100 K on a Bruker-AXS X8 Kappa diffractometer coupled to a Bruker APEX2 CCD detector using Cu K_{α}radiation (λ=1.54178 Å) from an IμS microsource. Data reduction was carried out with the program SAINT and semi-empirical absorption correction based on equivalents was performed with the program SADABS (J. Appl. Cryst. 2015, 48, 3-10). A summary of crystal properties and data/refinement statistics is given in Table 30.

TABLE 30
Crystal data and structure refinement for SJB-01

| Identification code | sfy177 | |
|---|---|---|
| Empirical formula | C₂₃ H₂₄ F N₃ O₂ | |
| Formula weight | 393.45 | |
| Temperature | 100(2) K | |
| Wavelength | 1.54178 Å | |
| Crystal system | Monoclinic | |
| Space group | P2₁ | |
| Unit cell dimensions | a = 12.3337(4) Å | α = 90°. |
| | b = 11.8844(4) Å | β = 111.8983(11)°. |
| | c = 14.8280(5) Å | γ = 90°. |

| | |
|---|---|
| Volume | 2016.65(12) Å³ |
| Z | 4 |
| Density (calculated) | 1.296 Mg/m³ |
| Absorption coefficient | 0.736 mm⁻¹ |
| F(000) | 832 |
| Crystal size | 0.460 × 0.410 × 0.215 mm³ |
| Theta range for data collection | 3.212 to 68.211°. |
| Index ranges | −14 <= h <= 14, −13 <= k <= 14, −17 <= l <= 17 |
| Reflections collected | 56094 |
| Independent reflections | 7322 [Rᵢₙₜ = 0.0289] |
| Completeness to theta = 67.679° | 100.0% |
| Absorption correction | Semi-empirical from equivalents |
| Refinement method | Full-matrix least-squares on F² |
| Data/restraints/parameters | 7322/5/540 |
| Goodness-of-fit on F² | 1.029 |
| Final R indices (I > 2σ(I)] | R1 = 0.0229, wR2 = 0.0613 |
| R indices (all data) | R1 = 0.0230, wR2 = 0.0613 |
| Absolute structure parameter | 0.016(16) |
| Extinction coefficient | 0.00186(19) |
| Largest diff. peak and hole | 0.159 and −0.143 e · Å⁻³ |

Structure Solution and Refinement

The structure was solved with direct methods using the program SHELXT (Acta Cryst. 2015, A71, 3-8) and refined against F² on all data with SHELXL (Acta Cryst. 2015, C71, 3-8) using established refinement techniques (Crystallography Reviews 2009, 15, 57-83). All non-hydrogen atoms were refined anisotropically. All carbon-bound hydrogen atoms were placed in geometrically calculated positions and refined using a riding model while constraining their Uᵢₛₒ to 1.2 times the U_{eq} of the atoms to which they bind (1.5 times for methyl groups). Coordinates for the hydrogen atoms on nitrogen were taken from the difference Fourier synthesis and those hydrogen atoms were subsequently refined semi-freely with the help of distance restraints on N—H distances (target values 0.88(2) Å for amide N—H and 0.91(2) Å for amine N—H). No further restraints were applied.

Crystal Structure

Compound SJB-01 crystallizes monoclinic chiral space group P2₁ with two crystallographically independent molecules of SJB-01 and no solvent in the asymmetric unit (FIG. 78).

In the crystal structure, the two crystallographically independent molecules of SJB-01 form a pseudo-centrosymmetric dimer that is held together by two bifurcated classical hydrogen bonds. Of those, two interactions are much stronger, namely N1-H1 . . . O4 and N4-H4 . . . O2, while the other two should be considered weak (N1-H1 . . . F2) and very weak (N4-H4 . . . F1). In addition, there are two intramolecular hydrogen bonds (N3-H3 . . . O2 and N6-H6 . . . O4), but the geometry of the resulting five-membered ring is somewhat tight. The packing is somewhat stabilized by three non-classical C—H . . . O interactions. The classical hydrogen bonds are drawn in FIG. 78 (although N6-H6 . . . O4 is hidden owing to the perspective of the molecules' orientation in FIG. 78) and all hydrogen bonds are listed in Table 31.

TABLE 31
Hydrogen bond parameters for SJB-01 [Å and °].

| D-H . . . A | d(D-H) | d(H . . . A) | d(D . . . A) | <(DHA) |
|---|---|---|---|---|
| N(1)—H(1) . . . F(2) | 0.895(17) | 2.460(19) | 3.1134(16) | 130.2(17) |
| N(1)—H(1) . . . O(4) | 0.895(17) | 2.018(18) | 2.8635(18) | 157.0(19) |
| N(3)—H(3) . . . O(2) | 0.920(17) | 2.23(2) | 2.6722(18) | 108.9(15) |
| N(4)—H(4) . . . F(1) | 0.874(17) | 2.556(19) | 3.2089(16) | 132.2(17) |
| N(4)—H(4) . . . O(2) | 0.874(17) | 2.203(18) | 3.0337(17) | 158.6(18) |
| N(6)—H(6) . . . O(4) | 0.900(17) | 2.25(2) | 2.6236(18) | 104.4(15) |
| C(16)—H(16) . . . O(1)#1 | 0.95 | 2.54 | 3.201(2) | 126.4 |
| C(23)—H(23B) . . . O(1)#2 | 0.98 | 2.45 | 3.339(2) | 151.3 |
| C(46)—H(46) . . . O(3)#3 | 0.95 | 2.34 | 3.231(2) | 155.8 |

Symmetry transformations used to generate equivalent atoms:
#1 −x + 1, y + ½, −z + 1
#2 −x + 1, y − ½, −z + 1
#3 −x + 1, y − ½, −z + 2

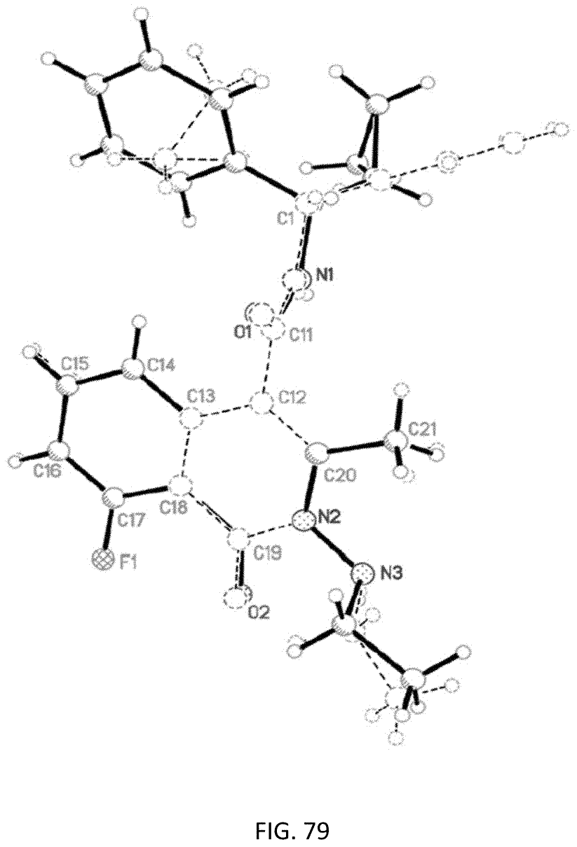

The two crystallographically independent molecules superimpose well. FIG. 79 shows an overlay calculated through all non-hydrogen atoms with exception of the phenyl- and cyclopropyl-groups on the chiral carbon atoms and also the nitrogen-bound ethyl group. For this overlay, the second molecule has been inverted. The calculated RMS deviation through all non-hydrogen atoms is 0.09 Å.

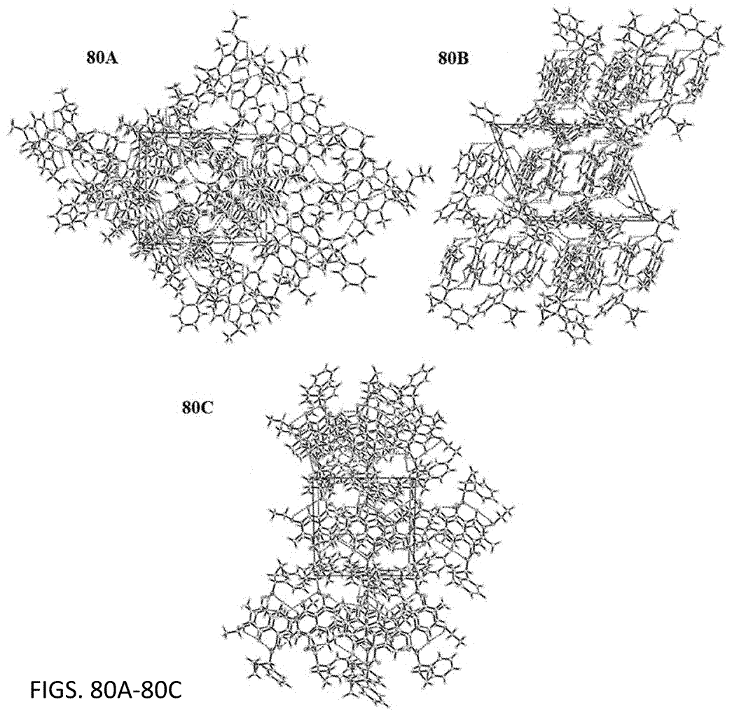

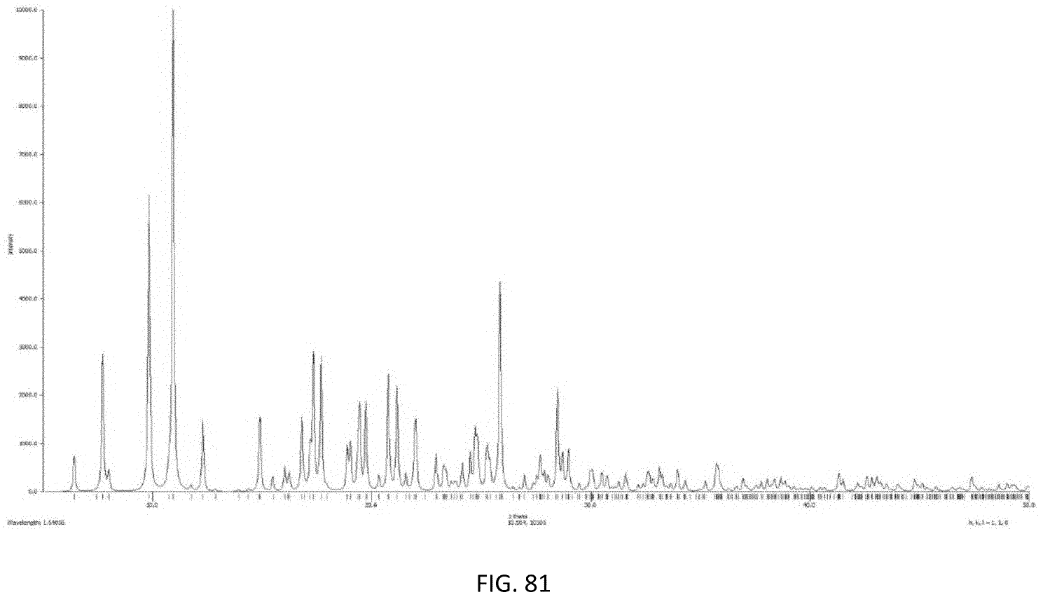

The molecule is chiral and the absolute structure could be determined based on resonant scattering signal. The Flack-x parameters as calculated by the Parsons method (Acta Cryst. 2004, A60, s61) refined to 0.016(16). Analysis of the anomalous signal using the method introduced by Hooft & Spek (J. Appl. Cryst. 2008, 41, 96-103) calculates the probability of the absolute structure to be correct to 1, the probability of the structure to be a racemic twin to 0 and the probability of the absolute structure to be incorrect to 0. Therefore, it can be determined with high confidence that the structure's chiral carbon atoms have the configuration C1:S in the first and C31:S in the second molecule. Both crystallographically independent molecules have the same configuration. FIG. 80 shows packing plots of the structure and FIG. 81 the simulated powder pattern.

### Example 5

### Preparation of SJB-01, Beta Form Polymorph

Input SJB-01 compound was synthesized. SJB-01 beta form may be obtained following crystallization utilizing various isolation processes. In certain embodiments, the HCl salt of the input API was used. In a preferred process, final deprotection of the base compound was completed in IPAc. The reaction solution was washed with water, 5% sodium bicarbonate in water, and water. The resulting IPAc layer was solvent swapped to acetone by distillation. n-Heptane was added to create a suspension of SJB-01 in 1:2 acetone:n-heptane. The suspension was heated to 55° C. to dissolve and cooled slowly to −20° C. and filtered. 7 kg of beta form polymorph SJB-01 was obtained using this described process.

### Example 6

### pH Solubility Studies of Beta Form Polymorph of SJB-01

Solubility in Buffered Solutions

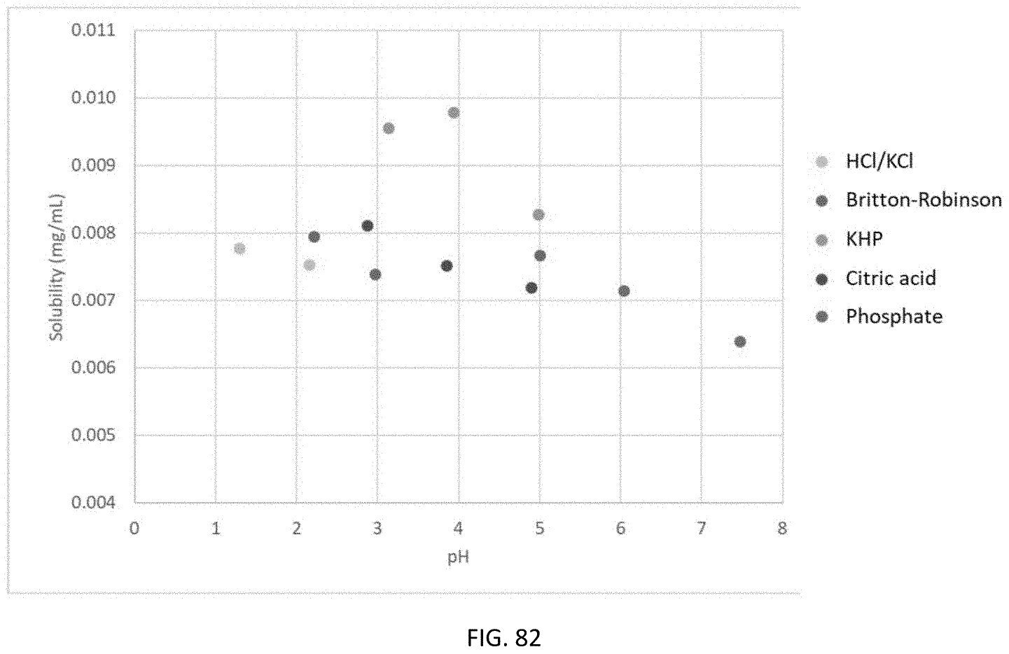

Aqueous buffers between pH 1.3 and 7.4 (13 total) were prepared and 3 mL added to about 15 mg of SJB-01 (L100149-44-1, jet-milled). These slurries were stirred on a hot-plate at 37° C. for 24 hours. Sonication and vortexting was employed within the first 2 hours of stirring to ensure thorough mixing and wetting of the solids as there was a clear tendency for them to clump and float at the liquid surface. After sonication and vortexing there was substantial solids suspended in solution (slurry) along with the solids that floated on top. At 24 hours, the samples were collected by syringe filtration of about 2 mL of solution. The first 1 mL of solution that passed through the filter was discarded and the second 0.5-1 mL of solution was used for HPLC analysis. The samples were injected into HPLC without dilution. Table 32 gives a summary of data and FIG. 82 shows a plot of solubility vs. pH.

The solubility profile with respect to pH generated was relatively flat. A trend of decreasing solubility with increase in pH was observed. The KHP buffered solutions showed a slightly higher solubility than other buffered solutions of similar pH (FIG. 82).

TABLE 32
Summary of data from the solubility vs. pH studies of SJB-01

| | | | | | | 24 h | | |
|---|---|---|---|---|---|---|---|---|
| | SJB-01 | Volume | Initial | | Ionic | | Solubility | Area |
| L1MA31003- | (mg) | (mL) | pH | Buffer | Strength | pH | (mg/mL) | (au) |
| 32-1 | 16.3 | 3 | 1.34 | HCl/KCl | 0.05M | 1.30 | 0.0078 | 369.96 |
| | 15.3 | 3 | 2.13 | HCl/KCl | 0.05M | 2.16 | 0.0075 | 358.18 |
| 32-3 | 15.1 | 3 | 2.21 | Britton-Robinson | 0.05M | 2.22 | 0.0079 | 378.41 |
| | | | | NaOH/AcOH/H₃PO₄/ | | | | |
| | | | | H₃BO₃/KCl | | | | |
| 32-4 | 14.9 | 3 | 3.18 | KHP/HCl | 0.05M | 3.14 | 0.0095 | 454.59 |
| 32-5 | 14.5 | 3 | 2.86 | Citric acid/NaOH | 0.05M | 2.88 | 0.0081 | 385.68 |
| 32-6 | 14.9 | 3 | 2.85 | Britton-Robinson | 0.05M | 2.97 | 0.0074 | 351.58 |
| | | | | NaOH/AcOH/H₃PO₄/ | | | | |
| | | | | H₃BO₃/KCl | | | | |
| 32-7 | 15.1 | 3 | 3.89 | KHP/HCl | 0.05M | 3.94 | 0.0098 | 465.60 |
| 32-8 | 15.0 | 3 | 3.82 | Citric acid/NaOH | 0.05M | 3.85 | 0.0075 | 357.94 |
| 32-9 | 14.4 | 3 | 4.96 | KHP/NaOH | 0.05M | 4.99 | 0.0083 | 393.73 |
| 32-10 | 14.9 | 3 | 4.90 | Citric acid/NaOH | 0.05M | 4.90 | 0.0072 | 342.03 |
| 32-11 | 16.1 | 3 | 4.95 | Britton-Robinson | 0.05M | 5.01 | 0.0077 | 364.80 |
| | | | | NaOH/AcOH/H₃PO₄/ | | | | |
| | | | | H₃BO₃/KCl | | | | |
| 32-12 | 15.1 | 3 | 5.98 | KH₂PO₄/NaOH | 0.05M | 6.04 | 0.0071 | 340.13 |
| 32-13 | 14.2 | 3 | 7.46 | KH₂PO₄/NaOH | 0.05M | 7.47 | 0.0064 | 304.56 |

HPLC Calibration Curve and Method

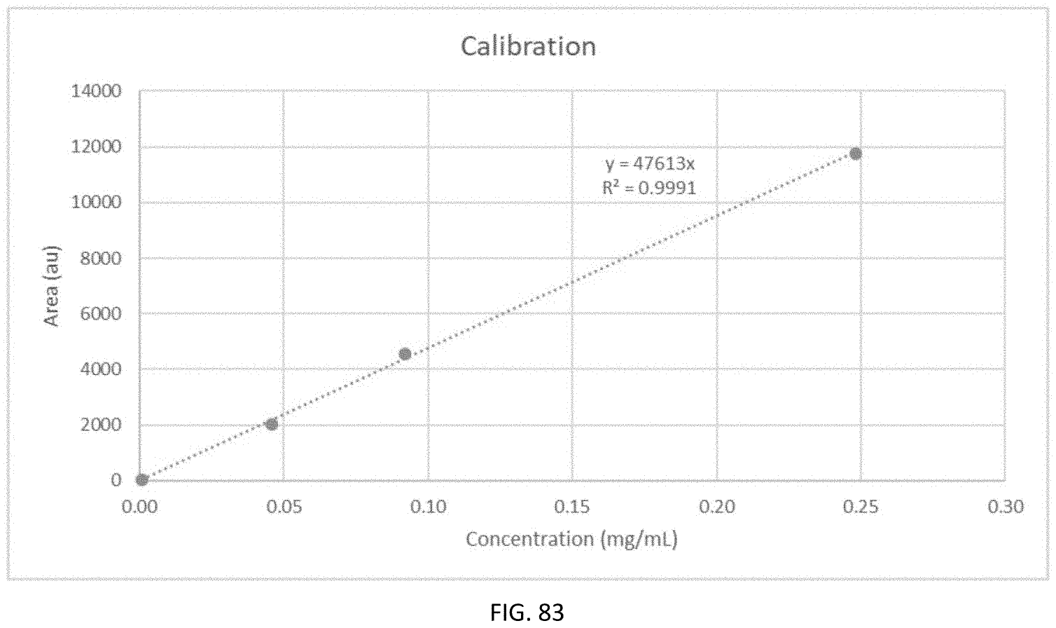

SJB-01 (L100149-44-1) was weighed directly into volumetric flasks and filled to volume with ACN:water (6:4 vol.) to build a calibration curve. Table 33 gives the data points and FIG. 83 shows the calibration curve. Details for the HPLC method used are given in Table 34; these follow the parameters used for previous dissolution study.

TABLE 33
Calibration samples used for relating the
HPLC peak area to concentration of SJB-01

| Calibration | | Concentration | HPLC |
|---|---|---|---|
| point | L1MA31003- | (mg/mL) | peak area |
| 1 | 33-1 | 0.001 | 42.92 |
| 2 | 33-2 | 0.046 | 2016.43 |
| 3 | 33-3 | 0.092 | 4571.70 |
| 4 | 33-4 | 0.248 | 11769.35 |

TABLE 34
HPLC method details

| Mobile Phase A | 0.05% TFA in distilled water | | |
|---|---|---|---|
| Mobile Phase B | 0.0375% TFA in ACN | | |
| Diluent | ACN:water (6:4 vol) | | |
| Injection Volume | 10 μL | | |
| Monitoring | 220 nm | | |
| Wavelength | | | |
| Column | Waters Cortex C18, 100 × 3 mm, 2.7 μm | | |
| Column Temperature | 40° C. | | |
| | Time | MP A | MP B |
| Gradient Method | (min) | (%) | (%) |
| | 0 | 95 | 5 |
| | 20 | 5 | 95 |
| | 22 | 5 | 95 |
| | 22.5 | 95 | 5 |
| | 25 | 95 | 5 |

Materials

| Reagent | Grade | Supplier | Catalog number | Lot number |
|---|---|---|---|---|
| HCl | ACS reagent, 37 wt. % in water | Millipore Sigma | 320331-500ML | SHBK1259 |
| NaOH | Certified | Fisher | S318-500 | 141711 |
| KCl | ACS reagent | Anachemia | 72830-300 | 410704 |
| AcOH | ACS reagent, >99.7% | Millipore Sigma | 695092-100ML | SHBJO239 |
| H₃PO₄ | ACS reagent, 85 wt. % in water | Millipore Sigma | 695017 | MKCF2007 |
| H₃BO₃ | ACS reagent, >99.5% | Millipore Sigma | B0394-100G | SLBT1445 |
| KHP | Bioxtra, >99.95% | Millipore Sigma | P1088-100G | MKB25705V |
| Citric acid | ACS reagent grade, >99.5% | Millipore Sigma | 251275-100G | MKBT2029V |
| Sodium citrate | Certified | Fisher | S279-500 | 127904 |
| dihydrate | | | | |
| KH₂PO₄ | For cell cultures, >99.0 | Millipore Sigma | P5655-500G | SLBK4244V |
| K₂HPO₄ | USP | Millipore Sigma | P2222-100G | SLBM7005V |

### Example 7

### Polymorphs in Clinical Trials

The beta form polymorph of the compound of formula (I) has been used in a Phase I clinical trial. During the clinical trial the beta form polymorph was administered to healthy subjects in a dosed suspension.

1. A beta form polymorph of a compound of formula (I): 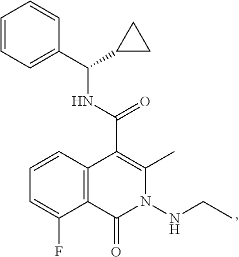 [SMILES: CCNn1c(C)c(C(=O)N[C@H](c2ccccc2)C2CC2)c2cccc(F)c2c1=O]
wherein the polymorph is a substantially pure polymorph.

2. The polymorph of claim 1, having an X-ray powder diffraction pattern comprising a peak at about 10.93° 2θ.

3. The polymorph of claim 1, having an X-ray powder diffraction pattern comprising peaks at about 7.80° and 10.93° 2θ.

4. The polymorph of claim 1, having an X-ray powder diffraction pattern comprising peaks at about 7.80°, 9.79°, 10.93°, 11.95°, 16.95°, 17.58°, 18.94°, 20.80°, 21.95°, 24.02°, 25.17°, and 28.25° 2θ.

5. The polymorph of claim 1, having an X-ray powder diffraction pattern substantially as shown in FIG. 38.

6. The polymorph of claim 1, having a differential scanning calorimetry thermogram comprising an endothermic peak at about 163° C.

7. The polymorph of claim 1, having a thermogravimetric analysis and differential scanning calorimetry thermogram substantially as shown in FIG. 39.

8. The polymorph of claim 1, wherein the polymorph has a solubility in water of 5 μg/mL.

9. An alpha form polymorph of a compound of formula (I): 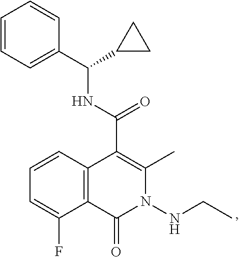 [SMILES: CCNn1c(C)c(C(=O)N[C@H](c2ccccc2)C2CC2)c2cccc(F)c2c1=O]
wherein the polymorph is a substantially pure polymorph.

10. (canceled)

11. (canceled)

12. (canceled)

13. The polymorph of claim 9, having an X-ray powder diffraction pattern substantially as shown in FIG. 33.

14. The polymorph of claim 9, having a differential scanning calorimetry thermogram comprising an endothermic peak at about 157° C.

15. The polymorph of claim 9, wherein the polymorph has a solubility in water of 6 μg/mL.

16. A gamma form polymorph of a compound of formula (I): 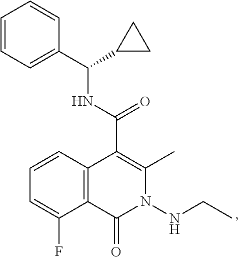 [SMILES: CCNn1c(C)c(C(=O)N[C@H](c2ccccc2)C2CC2)c2cccc(F)c2c1=O]
wherein the polymorph is a substantially pure polymorph.

17. (canceled)

18. (canceled)

19. (canceled)

20. The polymorph of claim 16, having an X-ray powder diffraction pattern substantially as shown in FIG. 47.

21. An amorphous form of a compound of formula (I): 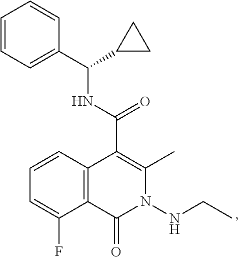 [SMILES: CCNn1c(C)c(C(=O)N[C@H](c2ccccc2)C2CC2)c2cccc(F)c2c1=O]
wherein the amorphous form is substantially pure.

22. The amorphous form of claim 21, having an X-ray powder diffraction pattern substantially as shown in FIG. 42.

23. The amorphous form of claim 21, having a thermogravimetric analysis and differential scanning calorimetry thermogram substantially as shown in FIG. 43.

24. The amorphous form of claim 21, wherein the amorphous form has a solubility in water of 5 μg/mL.

25. A pharmaceutical composition comprising the polymorph of claim 1 and a pharmaceutically acceptable carrier.

26.-33. (canceled)

34. A method of treating a subject in need thereof, comprising administering to the subject a polymorph of claim 1.

35.-50. (canceled)

51. Use of a polymorph of claim 1 in the manufacture of a medicament for the treatment of a disease.

52. A method of producing a polymorph of a compound of formula (I): 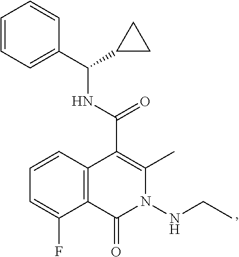 [SMILES: CCNn1c(C)c(C(=O)N[C@H](c2ccccc2)C2CC2)c2cccc(F)c2c1=O]
comprising: combining a compound of formula (I) and isopropyl acetate to form a first solution; distilling the first solution to replace the isopropyl acetate with acetone and to form a second solution; adding n-heptane to the second solution to form a suspension, wherein the acetone:n-heptane ratio is 1:2; heating the suspension to a temperature of 55° C. and slowly cooling the heated suspension to a temperature of −20° C. to form a composition comprising a beta form polymorph of the compound of formula (I).

53. (canceled)
